SEQUENCE LISTING (80 sequences)

SEQ ID NO: 1 - DNA, 2318 bp, Homo sapiens
  CDS (51)..(2147): GenBank Acc. No. NM_001063, transferrin gene and protein
gcacagaagcgagtccgactgtgctcgctgctcagcgccgcacccggaagatgaggctcgccgtgggagccctgctggtctgcgccgtcctggggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaaaatctcagaggtagggctgccaccaaggtgaagatgggaacgcagatgatccatgagtttgccctggtttcactggcccaagtggtttgtgctaaccacgtctgtcttcacagctctgtgttgccatgtgtgctgaacaaaaaataaaaattattattgattttatatttc

SEQ ID NO: 2 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 3 - protein, 679 aa, Homo sapiens
  MISC_FEATURE: Mature Transferrin Protein
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 4 - protein, 12 aa, Artificial sequence
  Neutrophil splice variant sequence
EDCIALKGEADA

SEQ ID NO: 5 - protein, 5 aa, Artificial sequence
  Pentapeptide capable of reducing inflammatory responses
XXXXX

SEQ ID NO: 6 - protein, 11 aa, Artificial sequence
  Peptide that blocks immune complex binding
PDARHSTTQPR

SEQ ID NO: 7 - protein, 8 aa, Homo sapiens
  misc_feature: Peptide derived from IgG1
KFNWYVDG

SEQ ID NO: 8 - protein, 8 aa, Homo sapiens
  misc_feature: Peptide derived from IgG1
KADWYVDG

SEQ ID NO: 9 - protein, 5 aa, Homo sapiens
  misc_feature: Peptide derived from IgM
EWMQR

SEQ ID NO: 10 - protein, 10 aa, Homo sapiens
  misc_feature: Peptide derived from IgG
GVQVHNAKTK

SEQ ID NO: 11 - protein, 11 aa, Homo sapiens
  misc_feature: Peptide derived from IgG
VQVHNAKTKPR

SEQ ID NO: 12 - protein, 16 aa, Homo sapiens
  misc_feature: Peptide derived from IgG
FNWYVDGVQVHNAKTK

SEQ ID NO: 13 - protein, 5 aa, Homo sapiens
  misc_feature: Peptide derived from IgG
FNWYV

SEQ ID NO: 14 - protein, 4 aa, Homo sapiens
  misc_feature: Peptide derived from IgG
TKPR

SEQ ID NO: 15 - protein, 23 aa, Artificial sequence
  Peptide able to nonspecifically activate lymphocytes
TISKAKGQPREPQVYTLPSREEM

SEQ ID NO: 16 - protein, 10 aa, Artificial sequence
  Peptide derived from IgG
YSKLTVDKSR

SEQ ID NO: 17 - protein, 7 aa, Artificial sequence
  Peptide derived from IgG
QYDSTYR

SEQ ID NO: 18 - protein, 5 aa, Artificial sequence
  Peptide derived from IgE
DSDPR

SEQ ID NO: 19 - protein, 6 aa, Artificial sequence
  Peptide able to block IgE-binding to IgE Fc receptors
PDARHS

SEQ ID NO: 20 - protein, 11 aa, Artificial sequence
  Sequence identical to portion of human IgE
VFSRLEVTRAE

SEQ ID NO: 21 - protein, 12 aa, Artificial sequence
  Peptide identical to portion of human IgE
PRKTKGSGFFVF

SEQ ID NO: 22 - DNA, 92 bp, Artificial sequence
  Sequence within pREX0004 contain ing linker
ctagataaaagggaagtgaaactggagctctggtggtggttctggtggtggttctggtggtggttctgatatcaacctggaagtgaaggtac

SEQ ID NO: 23 - DNA, 84 bp, Artificial sequence
  Sequence within pREX0004 containing linker
cttcacttccaggttgatatcagaaccaccaccagaaccaccaccagaaccaccaccagagctccagtttcacttcccttttat

SEQ ID NO: 24 - protein, 21 aa, Artificial sequence
  Linker peptide
SGGGSGGGSGGGSDINLEVKV

SEQ ID NO: 25 - DNA, 38 bp, Artificial sequence
  Mutagenic PCR primer
agcctgcactttccgtcgacctgaagtgaaactggaag

SEQ ID NO: 26 - DNA, 38 bp, Artificial sequence
  Mutagenic PCR primer
cagtcatgtctaagcttattacttcacttccaggttgg

SEQ ID NO: 27 - protein, 240 aa, Artificial sequence
  Anti-TNF alpha polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSSGGGSGGGSGGGSDILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 28 - DNA, 357 bp, Artificial sequence
  Vh DNA sequence
gaagtgaaactggaagaaagcggcggcggcctggtgcagccgggcggcagcatgaaactgagctgcgtggcgagcggctttatttttagcaaccattggatgaactgggtgcgtcagagcccggaaaaaggcctggaatgggtggcggaaattcgtagcaaaagcattaacagcgcgacccattatgcggaaagcgtgaaaggccgttttaccattagccgtgatgatagcaaaagcgcggtgtatctgcagatgaccgatctgcgtaccgaagataccggcgtgtattattgcagccgtaactattatggcagcacctatgattattggggccagggcaccaccctgaccgtgagc

SEQ ID NO: 29 - protein, 119 aa, Artificial sequence
  anti TNF-alpha VH
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVS

SEQ ID NO: 30 - DNA, 321 bp, Artificial sequence
  Vh DNA sequence
gatattctgctgacccagagcccggcgattctgagcgtgagcccgggcgaacgtgtgagctttagctgccgtgcgagccagtttgtgggcagcagcattcattggtatcagcagcgtaccaacggcagcccgcgtctgctgattaaatatgcgagcgaaagcatgagcggcattccgagccgttttagcggcagcggcagcggcaccgattttaccctgagcattaacaccgtggaaagcgaagatattgcggattattattgccagcagagccatagctggccgtttacctttggcagcggcaccaacctggaagtgaaa

SEQ ID NO: 31 - protein, 107 aa, Artificial sequence
  Anti TNFalpha sequence
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 32 - protein, 14 aa, Artificial sequence
  Peptide linker
SSGGGSGGGSGGGS

SEQ ID NO: 33 - DNA, 42 bp, Artificial sequence
  Peptide linker
tcttctggtggtggttctggtggtggttctggtggtggttct

SEQ ID NO: 34 - DNA, 42 bp, Artificial sequence
  Peptide linker (anti-sense)
agaaccaccaccagaaccaccaccagaaccaccaccagaaga

SEQ ID NO: 35 - protein, 45 aa, Artificial sequence
  N2 domain of human Tf
PEPRKPLEKAVANFFSGSCAPCADGTDFPQLCQLCPGCGCSTLNQ

SEQ ID NO: 36 - protein, 42 aa, Artificial sequence
  C1 domain of human Tf
NHCRFDEFFSEGCAPGSKKDSSLCKLCMGSGLNLCEPNNKEG

SEQ ID NO: 37 - protein, 47 aa, Artificial sequence
  N1 domain of human Tf
DKSKEFQLFSSPHGKDLLFKDSAHGFLKVPPRMDAKMYLGYEYVTAI

SEQ ID NO: 38 - protein, 49 aa, Artificial sequence
  C2 domain of huma Tf
NVTDCSGNFCLFRSETKDLLFRDDTVCLAKLHDRNTYEKYLGEEYVKAV

SEQ ID NO: 39 - protein, 5 aa, Artificial sequence
  VH CDR1 sequence
SYWIG

SEQ ID NO: 40 - protein, 17 aa, Artificial sequence
  VH CDR2 sequence
IIYPGDSDTRYSPSFQG

SEQ ID NO: 41 - protein, 7 aa, Artificial sequence
  VH CDR3 sequence
HGWGMDV

SEQ ID NO: 42 - protein, 5 aa, Artificial sequence
  P VH CDR1 sequence
NHWMN

SEQ ID NO: 43 - protein, 19 aa, Artificial sequence
  P VH CDR2 sequence
EIRSKSINSATHYAESVKG

SEQ ID NO: 44 - protein, 9 aa, Artificial sequence
  P VH CDR3 sequence
NYYGSTYDY

SEQ ID NO: 45 - protein, 5 aa, Artificial sequence
  33 CDR1 sequence
SYGMH

SEQ ID NO: 46 - protein, 17 aa, Artificial sequence
  33 CDR2 sequence
VISYDGSNKYYADSVKG

SEQ ID NO: 47 - protein, 9 aa, Artificial sequence
  33 CDR3 sequence
DSGDLAFDI

SEQ ID NO: 48 - protein, 5 aa, Artificial sequence
  35 CDR1 sequence
SFPIN

SEQ ID NO: 49 - protein, 17 aa, Artificial sequence
  35 CDR2 sequence
RIIPIIGIADYAQEFQG

SEQ ID NO: 50 - protein, 12 aa, Artificial sequence
  35 CDR3 sequence
PEAVTVPAPLDY

SEQ ID NO: 51 - protein, 5 aa, Artificial sequence
  37 CDR1 sequence
SYAIS

SEQ ID NO: 52 - protein, 11 aa, Artificial sequence
  37 CDR2 sequence
GTSNYAQKFQG

SEQ ID NO: 53 - protein, 17 aa, Artificial sequence
  37 CDR3 sequence
EVQFYHDSSGYLDALDI

SEQ ID NO: 54 - protein, 5 aa, Artificial sequence
  39 CDR1 sequence
TYVMN

SEQ ID NO: 55 - protein, 17 aa, Artificial sequence
  39 CDR2 sequence
GISGGGGSTYYADSVKG

SEQ ID NO: 56 - protein, 14 aa, Artificial sequence
  39 CDR3 sequence
DLSNRLSGGGTFDI

SEQ ID NO: 57 - protein, 14 aa, Artificial sequence
  VL CDR1 sequence
TGSSSNIGAGYDVH

SEQ ID NO: 58 - protein, 7 aa, Artificial sequence
  VL CDR2 sequence
YGNSNRP

SEQ ID NO: 59 - protein, 11 aa, Artificial sequence
  VL CDR3 sequence
QSYDSSLSGSV

SEQ ID NO: 60 - protein, 11 aa, Artificial sequence
  P VL CDR1 sequence
RASQFVGSSIH

SEQ ID NO: 61 - protein, 7 aa, Artificial sequence
  P VL CDR3 sequence
KYASESM

SEQ ID NO: 62 - protein, 9 aa, Artificial sequence
  P VL CDR3 sequence
QQSHSWPFT

SEQ ID NO: 63 - protein, 11 aa, Artificial sequence
  33 CDR1 sequence
RASQSVSSYLA

SEQ ID NO: 64 - protein, 7 aa, Artificial sequence
  33 CDR2 sequence
YDASNRA

SEQ ID NO: 65 - protein, 9 aa, Artificial sequence
  33 CDR3 sequence
LQRDNWPWT

SEQ ID NO: 66 - protein, 11 aa, Artificial sequence
  35 CDR1 sequence
RASQSISSWLA

SEQ ID NO: 67 - protein, 7 aa, Artificial sequence
  35 CDR2 sequence
YKASGLE

SEQ ID NO: 68 - protein, 8 aa, Artificial sequence
  35 CDR3 sequence
QQYNSYWT

SEQ ID NO: 69 - protein, 11 aa, Artificial sequence
  37 CDR1 sequence
RASQSLNNWLA

SEQ ID NO: 70 - protein, 7 aa, Artificial sequence
  37 CDR2 sequence
YKASSLE

SEQ ID NO: 71 - protein, 8 aa, Artificial sequence
  37 CDR3 sequence
QQYNSPWT

SEQ ID NO: 72 - protein, 11 aa, Artificial sequence
  39 CDR1 sequence
RASQSVSSYLA

SEQ ID NO: 73 - protein, 7 aa, Artificial sequence
  39 CDR2 sequence
NDASNRA

SEQ ID NO: 74 - protein, 9 aa, Artificial sequence
  39 CDR3 sequence
QQRSNWPLT

SEQ ID NO: 75 - DNA, 660 bp, Artificial sequence
  mTf sequence in pREX0080
agcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacgaattattatggttctacttatgattatgacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctc

SEQ ID NO: 76 - protein, 220 aa, Artificial sequence
  mTf sequence in pREX0080
KPVVAEFYGSKEDPQTFYYAVAVVKKDSGFQMNQLRGKKSCHTGLGRSAGWNIPIGLLYCDLPEPRKPLEKAVANFFSGSCAPCADGTNYYGSTYDYDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSP

SEQ ID NO: 77 - protein, 18 aa, Artificial sequence
  Amino acids 162-170 of mTf
ADGTNYYGSTYDYDFPQL

SEQ ID NO: 78 - DNA, 56 bp, Artificial sequence
  PCR primer P0109
ataatcataagtagaaccataataattcgtcccatccgcacaaggggcacagctgc

SEQ ID NO: 79 - DNA, 52 bp, Artificial sequence
  PCR primer P0110
gaattattatggttctacttatgattatgacttcccccagctgtgtcaactg

SEQ ID NO: 80 - DNA, 27 bp, Artificial sequence
  CDR3 from P VH
aattattatggttctacttatgattat

(19) United States
(12) Patent Application Publication
(10) Pub. No.: US 2003/0226155 A1
(43) Pub. Date: Dec. 4, 2003

(54) Modified transferrin-antibody fusion proteins

(75) Inventors: Homayoun Sadeghi, King of Prussia, PA (US); Christopher P. Prior, King of Prussia, PA (US); Andrew Turner, King of Prussia, PA (US)

Correspondence Address: MORGAN LEWIS & BOCKIUS LLP, 1111 PENNSYLVANIA AVENUE NW, WASHINGTON, DC, 20004, US

(73) Assignee: BIOREXIS PHARMACEUTICAL CORPORATION

(21) Appl. No.: 10/384,060

(22) Filed: Mar. 10, 2003

Related U.S. Application Data
(63) Continuation-in-part of application No. 10/231,494, filed on Aug. 30, 2002.
(60) Provisional application No. 60/315,745, filed on Aug. 30, 2001.
(60) Provisional application No. 60/334,059, filed on Nov. 30, 2001.
(60) Provisional application No. 60/406,977, filed on Aug. 30, 2002.

(51) Int. Cl. A01K 67/027; A61K 39/395; C07K 16/46

(52) U.S. Cl. 800/7; 530/391.1; 424/178.1

(57) ABSTRACT

Modified fusion proteins of transferrin and therapeutic proteins or peptides, preferably antibody variable regions, with increased serum half-life or serum stability are disclosed. Preferred fusion proteins include those modified so that the transferrin moiety exhibits no or reduced glycosylation, binding to iron and/or binding to the transferrin receptor.

### RELATED APPLICATIONS

This application is a Continuation-In-Part of U.S. application Ser. No. 10/231,494, filed Aug. 30, 2002, which claims the benefit of U.S. Provisional Application 60/315,745, filed Aug. 30, 2001 and U.S. Provisional Application 60/334,059, filed Nov. 30, 2001, all of which are herein incorporated by reference in their entirety. This application also claims the benefit of U.S. Provisional Application 60/406,977, filed Aug. 30, 2002, which is herein incorporated by reference in its entirety.

U.S. application entitled, “Modified Transferrin Fusion Proteins,” filed on March 2003, is also herein incorporated by reference in its entirety.

### FIELD OF THE INVENTION

The present invention relates to therapeutic proteins or peptides with extended serum stability and/or serum half-life fused to or inserted in a transferrin molecule modified to reduce or inhibit glycosylation, and/or reduce or inhibit iron binding and/or transferrin receptor binding. Specifically, the present invention includes single chain antibodies fused to or inserted in a transferrin molecule or a modified transferrin molecule.

### BACKGROUND OF THE INVENTION

Therapeutic proteins or peptides, including single chain antibodies, in their native state or when recombinantly produced are typically labile molecules exhibiting short periods of serum stability or short serum half-lives. In addition, these molecules are often extremely labile when formulated, particularly when formulated in aqueous solutions for diagnostic and therapeutic purposes.

Few practical solutions exist to extend or promote the stability in vivo or in vitro of proteinaceous therapeutic molecules. Polyethylene glycol (PEG) is a substance that can attach to a protein, resulting in longer-acting, sustained activity of the protein. If the activity of a protein is prolonged by the attachment to PEG, the frequency that the protein needs to be administered is decreased. PEG attachment, however, often decreases or destroys the protein's therapeutic activity.

Therapeutic proteins or peptides have also been stabilized by fusion to a heterologous protein capable of extending the serum half-life of the therapeutic protein. For instance, therapeutic proteins fused to albumin and antibody fragments may exhibit extended serum half-live when compared to the therapeutic protein in the unfused state. See U.S. Pat. Nos. 5,876,969 and 5,766,88.

Another serum protein, glycosylated human transferrin (Tf) has also been used to make fusions with therapeutic proteins to target delivery intracellularly or to carry heterologous agents across the blood-brain barrier. These fusion proteins comprising glycosylated human Tf have been used to target nerve growth factor (NGF) or ciliary neurotrophic factor (CNTF) across the blood-brain barrier by fusing full-length Tf to the therapeutic agent. See U.S. Pat. Nos. 5,672,683 and 5,977,307. In these fusion proteins, the Tf portion of the molecule is glycosylated and binds to two atoms of iron, which is required for Tf binding to its receptor on a cell and, according to the inventors of these patents, to target delivery of the NGF or CNTF moiety across the blood-brain barrier. Transferrin fusion proteins have also been produced by inserting an HIV-1 protease sequence into surface exposed loops of glycosylated transferrin to investigate the ability to produce another form of Tf fusion for targeted delivery to the inside of a cell via the Tf receptor (Ali et al. (1999) J. Biol. Chem. 274(34):24066-24073).

Serum transferrin (Tf) is a monomeric glycoprotein with a molecular weight of 80,000 daltons that binds iron for transport in the circulation to various tissues via the transferrin receptor (TfR) (Aisen et al. (1980) Ann. Rev. Biochem. 49: 357-393; MacGillivray et al. (1981) J. Biol. Chem. 258: 3543-3553, U.S. Pat. No. 5,026,651). Tf is one of the most common serum molecules, comprising up to about 5-10% of total serum proteins. Carbohydrate deficient transferrin occurs in elevated levels in the blood of alcoholics and exhibits a longer half life (approximately 14-17 days) than that of glycosylated transferrin (approximately 7-10 days). See van Eijk et al. (1983) Clin. Chim. Acta 132:167-171, Stibler (1991) Clin. Chem. 37:2029-2037 (1991), Arndt (2001) Clin. Chem. 47(1): 13-27 and Stibler et al. in “Carbohydrate-deficient consumption”, Advances in the Biosciences, (Ed Nordmann et al.), Pergamon, 1988, Vol. 71, pages 353-357).

The structure of Tf has been well characterized and the mechanism of receptor binding, iron binding and release and carbonate ion binding have been elucidated (U.S. Pat. Nos. 5,026,651, 5,986,067 and MacGillivray et al. (1983) J. Biol. Chem. 258(6):3543-3546).

Transferrin and antibodies that bind the transferrin receptor have also been used to deliver or carry toxic agents to tumor cells as cancer therapy (Baselga and Mendelsohn, 1994), and transferrin has been used as a non-viral gene therapy vector vehicle to deliver DNA to cells (Frank et al., 1994; Wagner et al., 1992). The ability to deliver proteins to the central nervous system (CNS) using the transferrin receptor as the entry point has been demonstrated with several proteins and peptides including CD4 (Walus et al., 1996), brain derived neurotrophic factor (Pardridge et al., 1994), glial derived neurotrophic factor (Albeck et al.), a vasointestinal peptide analogue (Bickel et al., 1993), a betaamyloid peptide (Saito et al., 1995), and an antisense oligonucleotide (Pardridge et al., 1995).

Transferrin fusion proteins have not, however, been modified or engineered to extend the serum half-life of a therapeutic protein or peptide or to increase bioavailability by reducing or inhibiting glycosylation of the Tf moiety or to reduce or prevent iron and/or Tf receptor binding.

Antibodies and their Structure

Antibodies which circulate in blood or other body fluids are termed humoral antibodies, as distinguished from “membrane antibodies” which remain bound to their parent lymphocytes. The term immunoglobulin is used to generically refer to all antibodies. In humans, all immunoglobulins are divided into five classes termed IgG, IgA, IgM, IgD and IgE. Each immunoglobulin molecule consists of two pairs of identical polypeptide chains, termed either heavy or light. The “heavy chains” are designated gamma (γ), alpha (α), mu (μ), delta (δ) and epsilon (ε). The “light chains” are designated lambda (λ) or kappa (κ).

Naturally occurring antibodies consist of four polypeptide chains: two identical heavy chains and two identical light chains. Each heavy chain is about 50-70 KDa, and each light chain is about 25 KDa. These chains are linked together by disulfide bonds. The basic structure of an antibody molecule has the shape of the letter Y. Each arm of the Y consists of one light chain and part of one heavy chain, while the stem of the Y consists of the rest of the heavy chain. The arm and the stem of the Y are held together by the hinge region which allows the arms to move.

The stem and a portion of the arm linked to the stem of the antibody molecule are made up of constant immunoglobulin domains. These domains have a conserved amino acid sequence and exhibit low variability. At the opposite ends of the arms are variable regions of the light and heavy chain consisting of 100 to 110 amino acids, within which are three small regions of non-conserved amino acid sequences or hyper-variable regions. These regions are responsible for antigen recognition and binding.

The domain structure of all light chains is identical regardless of the associated heavy chain class. Each light chain has two domains, one V_{L} domain and one domain with a relatively invariant amino acid sequence termed constant, light or C_{L}. Heavy chains, by contrast may have either three (IgG, IgA, IgD) or four (IgM, IgE) constant or C domains termed C_{H}1, C_{H}2, C_{H}3, and C_{H}4 and one variable domain, termed V_{H}. Alternatively, C domains may be designated according to their heavy chain class; thus Cε4 indicates the C_{H}⁴ domain of the IgE (ε) heavy chain.

Each variable light (V_{L}) and variable heavy (V_{H}) region contains three hypervariable regions known as the complementarity determining regions (CDRs). The CDRs come together to form a pocket for binding an antigen. As a result of the variability of the amino acid sequences in the hypervariable regions, the shape and properties of the binding sites vary, and the specificity of the sites for antigens vary.

Normally when an antigen enters a body, different parts of it are recognized by different naïve B cells. Each B cell forms antibodies with slightly different binding sites. Consequently, a mixture of antibody molecules is produced. In 1977, George Kohler and Cesar Milstein discovered a way to obtain large amounts of a single type of antibody with the same affinity. The method used by Kohler and Milstein to generate monoclonal antibodies involves fusing B cells from immunized animals with myeloma cells to generate a population of immortal hybridomas and selecting for the hybridoma that makes the desired antibody.

Monoclonal antibodies are important research tools and have been used as therapeutic agents. Monoclonal antibodies, however, are very expensive and difficult to produce. Additionally, their large size often inhibits them from reaching their target site.

Single Chain Antibody

Single chain antibodies (SCA) have been the subject of basic and applied research as a means to replace monoclonal antibodies in diagnostic and therapeutic applications. SCA are genetically engineered proteins having the binding specificity and affinity of monoclonal antibodies but are smaller in size which allow for more rapid capillary permeability. The advantages of SCA over monoclonal antibodies include greater tissue penetration for both diagnostic imaging and therapy, a decrease in immunogenic problems, more specific localization to target sites in the body, and easier and less costly to generate in large quantities.

SCA are usually formed using a short peptide linker to connect two variable regions of the V_{H} and V_{L} chains of an antibody. Suitable linkers for joining these variable regions are linkers which allow the V_{H} and V_{L} domains to fold into a single polypeptide chain having a three dimensional structure that maintains the binding specificity of a whole antibody. A description of the theory and production of single-chain antigen-binding proteins is found in Ladner et al., U.S. Pat. Nos. 4,946,778, 5,260,203, 5,455,030 and 5,518,889, and in Huston et al., U.S. Pat. No. 5,091,513 (“biosynthetic antibody binding sites” (BABS)), which disclosures are all incorporated herein by reference. The single-chain antigen-binding proteins produced under the process recited in the above patents have binding specificity and affinity substantially similar to that of the corresponding Fab fragment.

Fc Region

When antibodies are exposed to proteolytic enzymes such as papain or pepsin, several major fragments are produced. The fragments which retain antigen binding ability consist of the two “arms” of the antibody's Y configuration and are termed Fab (fragment-antigen binding) or Fab′2 which represent two Fab arms linked by disulfide bonds. The other major fragment produced constitutes the single “tail” or central axis of the Y and is termed Fc (fragment-crystalline) for its propensity to crystallize from solution. The Fc fragment of IgG, IgA, IgM, and IgD consists of dimers of the two carboxy terminal domains of each antibody (i.e., C_{H}2 and C_{H}3 in IgG, IgA, and IgD, and C_{H}3 and C_{H}4 in IgM). The IgE Fc fragment, by contrast, consists of a dimer of its three carboxy-terminal heavy chain domains (C_{ε}2, C_{ε}3 and C_{ε}4).

The Fc fragment contains the antibody's biologically “active sites” which enable the antibody to “communicate” with other immune system molecules or cells and thereby activate and regulate immune system defensive functions. Such communication occurs when active sites within antibody regions bind to molecules termed Fc receptors.

Fc receptors are molecules which bind with high affinity and specificity to molecular active sites with immunoglobulin Fc regions. Fc receptors may exist as integral membrane proteins within a cell's outer plasma membrane or may exist as free, “soluble” molecules which freely circulate in blood plasma or other body fluids.

Each of the five antibody classes have several types of Fc receptors which specifically bind to Fc regions of a particular class and perform distinct functions. Thus IgE Fc receptors bind with high affinity to only IgE Fc regions or to isolated IgE Fc fragments. It is known that different types of class specific Fc receptors exist which recognize and bind to different locations within the Fc region. For example, certain IgG Fc receptors bind exclusively to the second constant domain of IgG (C_{H}2), while Fc receptors mediating other immune functions bind exclusively to IgG's third constant domain (C_{H}3). Other IgG Fc receptors bind to active sites located in both C_{H}2 and C_{H}3 domains and are unable to bind to a single, isolated domain.

Once activated by antibody Fc region active sites, Fc receptors mediate a variety of important immune killing and regulatory functions. Certain IgG Fc receptors, for example, mediate direct killing of cells to which antibody has bound via its Fab arms (antibody—dependent cell mediate cytotoxicity—(ADCC)). Other IgG Fc receptors, when occupied by IgG, stimulate certain white blood cells to engulf and destroy bacteria, viruses, cancer cells or other entities by a process known as phagocytosis. Fc receptors on certain types of white blood cells known as B lymphocytes regulate their growth and development into antibody-secreting plasma cells. Fc receptors for IgE located on certain white cells known as basophils and mast cells, when occupied by antigen bridged IgE, trigger allergic reactions characteristic of hayfever and asthma.

Certain soluble Fe receptors which are part of the blood complement system trigger inflammatory responses able to kill bacteria, viruses and cancer cells. Other Fc receptors stimulate certain white blood cells to secrete powerful regulatory or cytotoxic molecules known generically as lymphokines which aid in immune defense. These are only a few representative examples of the immune activities mediated by antibody Fc receptors.

Most of the amino acids which make up antibodies function are molecular “scaffolding” which determine the antibody's structure, a highly regular three dimensional shape. It is this scaffolding which performs the critical function of properly exposing and spatially positioning antibody active sites which consist of several amino acid clusters. A particular active site, depending upon its function, may already be exposed and, therefore, able to bind to cellular receptors. Alternatively, a particular active site may be hidden until the antibody binds to an antigen, whereupon the scaffolding changes orientation and subsequently exposes the antibody's active site. The exposed active site then binds to its specific Fc receptor located either on a cell's surface or as part of a soluble molecule (e.g., complement) and subsequently triggers a specific immune activity.

Since the function of an antibody's scaffolding is to hold and position its active sites for binding to cells or soluble molecules, the antibody's active sites, when isolated and synthesized as peptides, can perform the immunoregulatory functions of the entire antibody molecule.

Depending upon the particular type of Fc receptor to which an active site peptide binds, the peptide may either stimulate or inhibit immune functions. Stimulation may occur if the Fc receptor is of the type that becomes activated by the act of binding to an Fc region or, alternatively, if an Fc active site peptide stimulates the receptor. The type of stimulation produced may include, but is not limited to, functions directly or indirectly mediated by antibody Fc region-Fc receptor binding. Examples of such functions include, but are not limited to, stimulation of phagocytosis by certain classes of white blood cells (polymorphonuclear neutrophils, monocytes and macrophages); macrophage activation; antibody dependent cell mediated cytotoxicity (ADCC); natural killer (NK) cell activity; growth and development of B and T lymphocytes and secretion by lymphocytes of lymphokines (molecules with killing or immunoregulatory activities).

### SUMMARY OF THE INVENTION

As described in more detail below, the present invention includes modified Tf fusion proteins comprising at least one antibody or CDR fragment, preferably an antibody variable region, wherein the Tf portion is engineered to extend the serum half-life or bioavailability of the molecule. The invention also includes pharmaceutical formulations and compositions comprising the fusion proteins, methods of extending the serum stability, serum half-life and bioavailability of an antibody or CDR fragment by fusion to modified transferrin, nucleic acid molecules encoding the modified Tf fusion proteins, and the like. Another aspect of the present invention relates to methods of treating a patient with a modified Tf fusion protein.

Preferably, the modified Tf fusion proteins comprise a human transferrin Tf moiety that has been modified to reduce or prevent glycosylation and/or iron and receptor binding.

In one aspect, the present invention provides trans-bodies comprising SCA or CDR regions linked to transferrin or modified transferrin. The trans-bodies can be constructed using different antibody variable regions for various pharmacological and diagnostic applications.

In another aspect, the present invention provides trans-bodies that comprise one or more antigenic peptides and antibody variable regions fused to transferrin or modified transferrin. These trans-bodies not only have the ability to bind to antigens but also to induce immune response in a host. The present invention also provides trans-bodies comprising one or more antigen binding peptides.

Further, the trans-bodies of the present invention comprise antibodies against toxins fused to transferrin or modified transferrin molecule. Examples of toxins include but are not limited to Clostridium botulinum, Clostridium difficile, Clostridium tetani, and Bacillus anthracis.

### BRIEF DESCRIPTION OF THE DRAWINGS

FIG. 1 shows an alignment of the N and C Domains of Human (Hu) transferrin (Tf) with similarities and identities highlighted.

FIGS. 2A-2B show an alignment of transferrin sequences from different species. Light shading: Similarity; Dark shading: Identity

FIG. 3 shows the location of a number of Tf surface exposed insertion sites for therapeutic proteins, polypeptides or peptides.

FIGS. 4A-4B show the V_{H} and V_{L} regions for a number of preferred anti-TNFα antibodies used to produce modified Tf fusion proteins.

### DETAILED DESCRIPTION

General Description

The present invention is based in part on the finding by the inventors that antibodies, antibody fragments, CDR regions, and SCA can be stabilized to extend their serum half-life and/or activity in vivo by genetically fusing SCA to transferrin, modified transferrin, or a portion of transferrin or modified transferrin sufficient to extend the half-life of the molecule in serum. The modified transferrin fusion proteins include a transferrin protein or domain covalently linked to an SCA antibody or antibody fragment, wherein the transferrin portion is modified to contain one or more amino acid substitutions, insertions or deletions compared to a wild-type transferrin sequence. In one embodiment, Tf fusion proteins are engineered to reduce or prevent glycosylation within the Tf or a Tf domain. In other embodiments, the Tf protein or Tf domain(s) is modified to exhibit reduced or no binding to iron or carbonate ion, or to have a reduced affinity or not bind to a Tf receptor (TfR).

In one embodiment, the present invention provides a fusion protein comprising variable regions of antibodies fused to or inserted into a transferrin or modified transferrin. Specifically, the present invention is based in part on the use of transferrin or modified transferrin to connect at least two variable regions of an antibody to form a modified form of a SCA. The SCA fusion protein formed in this manner has the ability of binding the antigen of interest and has the long circulating half-life of transferrin.

Usually, SCA are made by connecting two variable regions with a short peptide. This peptide can have any sequence and is often chosen mostly for its three dimensional structure rather than its sequence homology or biological function. However, since the peptide is an unnatural product, it induces immune reactions. Unlike the short peptide, transferrin is a naturally occurring protein and is not antigenic. SCA formed by using transferrin as a linker are a type of trans-body, i.e. transferrin with antibody activity. Trans-bodies are pharmaceutically useful and easy to make in a microbial system, such as yeast. Additionally, the large and soluble transferrin backbone helps solubilize and stabilize the variable domains attached to it. Trans-bodies can be constructed using a variety of variable regions and used for various pharmacological and diagnostic applications.

The present invention therefore includes trans-bodies, therapeutic compositions comprising the trans-bodies, and methods of treating, preventing, or ameliorating diseases or disorders by administering the trans-bodies. A trans-body of the invention includes at least an antibody variable domain and at least a fragment or variant of modified transferrin, which are associated with one another, preferably by genetic fusion (i.e., the trans-body is generated by translation of a nucleic acid in which a polynucleotide encoding all or a portion of the antibody variable domain is joined in-frame with a polynucleotide encoding all or a portion of modified transferrin). In a preferred embodiment, the present invention provides trans-bodies comprising antibody variable regions selected from the group consisting of V_{H}, V_{L}, or one or more CDR regions. The antibody variable region and transferrin protein, once part of the transferrin fusion protein, may be referred to as a “portion”, “region” or “moiety” of the transferrin fusion protein (e.g., a “SCA or antibody variable region portion” or a “transferrin protein portion”).

In one embodiment, the invention provides a trans-body comprising, or alternatively consisting of, an antibody variable region and a transferrin or a modified transferrin protein. In other embodiments, the invention provides a trans-body comprising, or alternatively consisting of, a biologically active antibody variable region and a transferrin or modified transferrin protein. In other embodiments, the invention provides a trans-body comprising, or alternatively consisting of, a biologically active and/or therapeutically active variant of an antibody variable region, for example a humanized antibody variable region, and a transferrin or modified transferrin protein. In further embodiments, the invention provides a trans-body comprising an antibody variable region, and a biologically active and/or therapeutically active fragment of modified transferrin.

Additionally, the present invention discloses trans-bodies comprising at least one antigenic peptide or immunomodulatory peptide. Such trans-bodies are not only able to bind their antigens but also can induce immune responses in the host.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Definitions

As used herein, the term “antibody variable region” comprises one or more V_{H}, V_{L}, or CDR region.

As used herein, the term “trans-bodies” refers to transferrin with antibody activity. Preferably, a trans-body comprises at least one antibody variable region and a transferrin molecule, modified transferrin molecule, or a fragment thereof. Trans-bodies may additionally comprise one or more antigenic peptides that are capable of inducing an immune response in a host.

As used herein, the term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Antibodies may exist as intact immunoglobulins, or as modifications in a variety of forms including, for example, an Fv fragment containing only the light and heavy chain variable regions, a Fab or (Fab)′₂ fragment containing the variable regions and parts of the constant regions, a single-chain antibody (Bird et al., Science 242: 424-426 (1988); Huston et al., Proc. Natl. Acad. Sci. USA 85: 5879-5883 (1988) both incorporated by reference herein), and the like. The antibody may be of animal (especially mouse or rat) or human origin or may be chimeric (Morrison et al., Proc Natl. Acad. Sci. USA 81, 6851-6855 (1984) incorporated by reference herein) or humanized (Jones et al., Nature 321, 522-525 (1986), and published UK patent application #8707252, both incorporated by reference herein). As used herein the term “antibody” includes these various forms.

The term “single chain variable fragments of antibodies” (scFv) or “single chain antibody” (SCA) as used herein means a polypeptide containing a V_{L} domain linked to a V_{H} domain by a peptide linker (L), represented by V_{L}-L-V_{H}. The order of the V_{L} and V_{H} domains can be reversed to obtain polypeptides represented as V_{H}-L-V_{L}. “Domain” or “region” is a segment of protein that assumes a discrete function, such as antigen binding or antigen recognition.

As used herein, the term “multivalent single chain antibody” means two or more single chain antibody fragments covalently linked by a peptide linker. The antibody fragments can be joined to form bivalent single chain antibodies having the order of V_{L} and V_{H} domains as follows: V_{L}-L-V_{H}-L-V_{L}-L-V_{H}; V_{L}-L-V_{H}-L-V_{H}-L-V_{L}; V_{H}-L-V_{L}-L-V_{H}-L-V_{L}; or V_{H}-L-V_{L}-L-V_{L}-L-V_{H}. Single chain multivalent antibodies which are trivalent and greater have one or more antibody fragments joined to a bivalent single chain antibody by an additional interpeptide linker. In a preferred embodiment, the number of V_{L} and V_{H} domains is equivalent.

As used herein, “Fv” region refers to a single chain antibody Fv region containing a variable heavy (V_{H}) and a variable light (V_{L}) chain. The heavy and light chain may be derived from the same antibody or different antibodies thereby producing a chimeric Fv region.

As used herein, the term “hypervariable region” refers to the amino acid residues of an antibody which are responsible for antigen-binding. The hypervariable region comprises amino acid residues from a “complementarity determining region” or “CDR” (i.e. about residues 24-34 (L1), 50-56 (L2) and 89-97 (L3) in the light chain variable domain and about residues 31-35 (H1), 50-65 (H2) and 95-102 (H3) in the heavy chain variable domain (Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and about residues 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

As is well-known in the art, the complementarity determining regions (CDRs) of an antibody are the portions of the antibody which are largely responsible for antibody specificity. The CDR's directly interact with the epitope of the antigen (see, in general, Clark, 1986; Roitt, 1991). In both the heavy chain and the light chain variable regions of IgG immunoglobulins, there are four framework regions (FR1 through FR4) separated respectively by three complementarity determining regions (CDR1 through CDR3). The framework regions (FRs) maintain the tertiary structure of the paratope, which is the portion of the antibody which is involved in the interaction with the antigen. The CDRs, and in particular the CDR3 regions, and more particularly the heavy chain CDR3 contribute to antibody specificity. Because these CDR regions and in particular the CDR3 region confer antigen specificity on the antibody these regions may be incorporated into trans-bodies to confer the identical antigen specificity onto that entity.

The sequence of the CDR regions, for use in synthesizing trans-bodies of the invention, may be determined by methods known in the art. The heavy chain variable region is a peptide which generally ranges from 100 to 150 amino acids in length. The light chain variable region is a peptide which generally ranges from 80 to 130 amino acids in length. The CDR sequences within the heavy and light chain variable regions which include only approximately 3-25 amino acid sequences may easily be sequenced by one of ordinary skill in the art. The peptides may even be synthesized by commercial sources such as by the Scripps Protein and Nucleic Acids Core Sequencing Facility (La Jolla Calif.).

In other embodiments, CDR regions or sequences may be randomly generated as a library of peptide sequences and screened using standard arrays for the desired binding or functional property. The sequences of the framework regions of different light or heavy chains are relatively conserved within a species. As used herein, a “human framework region” is a framework region that is substantially identical (about 85% or more, usually about 90-95% or more) to the framework region of a naturally occurring human immunoglobulin. The framework region of an antibody, that is the combined framework regions of the constituent light and heavy chains, serves to position and align the CDR's.

As used herein, the term “binding domain” refers to one or a combination of the following: (a) a V_{L} plus a V_{H} region of an immunoglobulin (IgG, IgM or other immunoglobulin); (b) a V_{L} plus V_{L} region of an immunoglobulin (IgG, IgM or other immunoglobulin); (c) a V_{H} plus V_{H} region of an immunoglobulin (IgG, IgM or other immunoglobulin); (d) a single V_{L} region of an immunoglobulin (IgG, IgM or other immunoglobulin); (e) a single V_{H} region of an immunoglobulin (IgG, IgM or other immunoglobulin) or one or more CDR peptide sequences; or (f) a peptide which has an antigen binding activity similar to a CDR peptide.

As used herein, the term “humanized” refers to forms of non-human (e.g. murine) antibodies which are specific chimeric immunoglobulins, immunoglobulin chains, or fragments thereof (such as Fv, Fab, Fab′, F(ab′)₂ or other antigen-binding subsequences of antibodies) and which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues which are found neither in the recipient antibody or the donor antibody. These modifications are made to further refine and optimize antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin.

As used herein, the term “biological activity” refers to a function or set of activities performed by a therapeutic molecule, protein or peptide, preferably an antibody variable fragment or CDR region, in a biological context (i.e., in an organism or an in vitro facsimile thereof). Biological activities may include but are not limited to the functions of the antibody portion of the claimed fusion proteins. A fusion protein or peptide of the invention is considered to be biologically active if it exhibits one or more biological activities of an antibody counterpart or exerts a discernable response in an in vivo or in vitro assay relevant to the trans-body being tested.

As used herein, an “amino acid corresponding to” or an “equivalent amino acid” in a sequence is identified by alignment to maximize the identity or similarity between a first sequence and at least a second sequence. The number used to identify an equivalent amino acid in a second sequence is based on the number used to identify the corresponding amino acid in the first sequence. In certain cases, these phrases may be used to describe the amino acid residues in human transferrin compared to certain residues in rabbit serum transferrin or transferrin from another species.

As used herein, the terms “fragment of a Tf protein” or “Tf protein,” or “portion of a Tf protein” refer to an amino acid sequence comprising at least about 5%, 10%, 20%, 30%, 40%, 50%, 60%, 70%, 80%, 90%, 95%, 96%, 97%, 98%, 99% or 100% of a naturally occurring Tf protein or mutant thereof.

As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, a “heterologous polynucleotide” or a “heterologous nucleic acid” or a “heterologous gene” or a “heterologous sequence” or an “exogenous DNA segment” refers to a polynucleotide, nucleic acid or DNA segment that originates from a source foreign to the particular host cell, or, if from the same source, is modified from its original form. A heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. As an example, a signal sequence native to a yeast cell but attached to a human Tf sequence is heterologous.

As used herein, an “isolated” nucleic acid sequence refers to a nucleic acid sequence which is essentially free of other nucleic acid sequences, e.g., at least about 20% pure, preferably at least about 40% pure, more preferably about 60% pure, even more preferably about 80% pure, most preferably about 90% pure, and even most preferably about 95% pure, as determined by agarose gel electrophoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

As used herein, two or more DNA coding sequences are said to be “joined” or “fused” when, as a result of in-frame fusions between the DNA coding sequences, the DNA coding sequences are translated into a polypeptide fusion. The term “fusion” in reference to Tf fusions includes, but is not limited to, attachment of at least one therapeutic protein, polypeptide or peptide, preferably an antibody variable region, to the N-terminal end of Tf, attachment to the C-terminal end of Tf, and/or insertion between any two amino acids within Tf.

As used herein, the term “modified transferrin” as used herein refers to a transferrin molecule that exhibits at least one modification of its amino acid sequence, compared to wildtype transferrin. In a preferred embodiment, “modified transferrin” refers to transferrin that has been modified to exhibit reduced or no glycosylation, reduced or no iron or carbonate binding, and reduced or no transferrin receptor binding.

As used herein, the term “modified transferrin fusion protein” as used herein refers to a protein formed by the fusion of at least one molecule of modified transferrin (or a fragment or variant thereof) to at least one molecule of a therapeutic protein (or fragment or variant thereof), preferably an antibody variable fragment or CDR.

As used herein, the terms “nucleic acid” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

As used herein, a DNA segment is referred to as “operably linked” when it is placed into a functional relationship with another DNA segment. For example, DNA for a signal sequence is operably linked to DNA encoding a fusion protein of the invention if it is expressed as a preprotein that participates in the secretion of the fusion protein; a promoter or enhancer is operably linked to a coding sequence if it stimulates the transcription of the sequence. Generally, DNA sequences that are operably linked are contiguous, and in the case of a signal sequence or fusion protein both contiguous and in reading phase. However, enhancers need not be contiguous with the coding sequences whose transcription they control. Linking, in this context, is accomplished by ligation at convenient restriction sites or at adapters or linkers inserted in lieu thereof.

As used herein, the term “promoter” refers to a region of DNA involved in binding RNA polymerase to initiate transcription.

As used herein, the term “recombinant” refers to a cell, tissue or organism that has undergone transformation with recombinant DNA.

As used herein, a targeting entity, protein, polypeptide or peptide refers to such molecules that binds specifically to a particular cell type [normal (e.g., lymphocytes) or abnormal e.g., (cancer cell)] and therefore may be used to target a trans-body or compound (drug, or cytotoxic agent) to that cell type specifically.

As used herein, “therapeutic protein” induces proteins, polypeptides, antibodies, SCA, antibody variable fragments, CDRs or peptides or fragments or variants thereof, having one or more therapeutic and/or biological activities. The terms peptides, proteins, and polypeptides are used interchangeably herein. Additionally, the term “therapeutic protein” may refer to the endogenous or naturally occurring correlate of a therapeutic protein. By a polypeptide displaying a “therapeutic activity” or a protein that is “therapeutically active” is meant a polypeptide that possesses one or more known biological and/or therapeutic activities associated with a therapeutic protein such as one or more of the therapeutic proteins described herein or otherwise known in the art. As a non-limiting example, a “therapeutic protein” is a protein that is useful to treat, prevent or ameliorate a disease, condition or disorder. Such a disease, condition or disorder may be in humans or in a non-human animal, e.g., veterinary use.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

As used herein, the term “transformant” refers to a cell, tissue or organism that has undergone transformation.

As used herein, the term “transgene” refers to a nucleic acid that is inserted into an organism, host cell or vector in a manner that ensures its function.

As used herein, the term “transgenic” refers to cells, cell cultures, organisms, bacteria, fungi, animals, plants, and progeny of any of the preceding, which have received a foreign or modified gene and in particular a gene encoding a modified Tf fusion protein by one of the various methods of transformation, wherein the foreign or modified gene is from the same or different species than the species of the organism receiving the foreign or modified gene.

“Variants or variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide. As used herein, “variant”, refers to a therapeutic protein portion of a transferrin fusion protein of the invention, differing in sequence from a native therapeutic protein but retaining at least one functional and/or therapeutic property thereof as described elsewhere herein or otherwise known in the art.

As used herein, the term “vector” refers broadly to any plasmid, phagemid or virus encoding an exogenous nucleic acid. The term is also be construed to include non-plasmid, non-phagemid and non-viral compounds which facilitate the transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994). Examples of non-viral vectors include, but are not limited to, liposomes, polyamine derivatives of DNA, and the like.

As used herein, the term “wild type” refers to a polynucleotide or polypeptide sequence that is naturally occurring.

As used herein the term “toxin” refers to a poisonous substance of biological origin.

As used herein, the term “immunomodulatory” refers to an ability to increase or decrease an antigen-specific immune response, either at the B cell or T cell level. Immunomodulatory activity can be detected e.g., in T cell proliferation assays, by measurement of antibody production, lymphokine production or T cell responsiveness. In particular, in addition to affects on T cell responses, the immunomodulatory polypeptides of the invention may bind to immunoglobulin (i.e., antibody) molecules on the surface of B cells, and affect B cell responses as well.

As used herein, the term “immunomodulatory peptide” is a peptide that affects immune response.

As used herein, the term “Fc region” refers to the stalk of the antibody molecule composed of constant regions. The Fc region is also called the effector region. The Fc region interacts with other components of the immune system, transducing the signal of bacterial presence into cellular response. The Fc region of the antibody is the important region in creating different readout over the course of an immune response. This region is composed of heavy chains, and the way in which the readout is changed over the course of an immune response is to change the structure of the Fc region of the antibody. By changing the constant region, one changes the class of antibody. This process is called Class Switching, and occurs in the B Lymphocytes.

Single Chain Antibodies and Trans-Bodies

Single chain compared to conventional antibodies are smaller in size and significantly reduced cost to generate. The smaller size of single chain antibodies may reduce the body's immunologic reaction and thus increase the safety and efficacy of therapeutic applications. Conversely, single chain antibodies could be engineered to be highly antigenic.

Various single chain antibodies (SCA) were originally invented to simplify antibody selection and production. However, they prove to be of limited or no therapeutic value due to their small size, self-aggregation, and short in vivo half-life. Addition of transferrin to SCA significantly increases the in vivo half-life, stability, and ease of manufacture of SCA.

Thus components from SCA can be fused to the N-, C- or N-, and C-termini of transferrin or modified transferrin (V_{L}, V_{H} and/or one or more CDR regions). These fusions could also be carried out using different parts or domains of transferrin such as the N domain or C domain. The proteins could be fused directly or using a linker peptide of various length. It is also possible to fuse all or part of the active SCA within the scaffold of transferrin. In such instances the fusion protein is made by inserting the cDNA of the SCA within the cDNA of transferrin for production of the protein in cells.

In one embodiment, two V_{H} or two V_{L} regions could be attached to the two ends of or inserted into transferrin or modified transferrin. In another embodiment, one V_{H} and one V_{L} could be attached to or inserted in transferrin or modified transferrin. The variable regions could be connected to each other through a linker (L) and then fused to or inserted into transferrin. The linker is a molecule that is covalently linked to the variable domains for ease of attachment to or insertion into Tf. Together the linker and Tf provides enough spacing and flexibility between the two domains such that they are able to achieve a conformation in which they are capable of specifically binding the epitope to which they are directed. Additionally, transferrin can be modified so that the variable regions attached to the two termini can come close together. Examples of such modification include but are not limited to removal of C-terminus proline and/or the cystine loop close to the C-terminus of Tf to give more flexibility.

The present invention also contemplates multivalent trans-bodies. Antibody variable regions having the order of V_{H}-L-V_{H} could be fused to one end of the transferrin and variable regions having the order V_{L}-L-V_{L} could be fused to the same transferrin at the other terminus. Other sequences of variable regions forming multivalent SCA are also contemplated by the present invention. Examples include, but are not limited to, V_{H}-L-V_{L} and V_{L}-L-V_{H} and those having more variable domains linked together. The variable regions and linkers could also be inserted into the transferrin molecule.

Alternatively, the multivalent antibody variable regions can be formed by inserting variable domains in the transferrin or modified transferrin molecule without using any nonnatural peptide linkers. In this way, the portions of the transferrin molecule act as linkers to provide spacing and flexibility between the variable domains.

In one aspect of the invention, the variable regions binding the same antigen can be fused to the different termini of the same transferrin or modified transferrin molecule. In another aspect of the invention, variable regions that bind different antigens can be fused to the different termini of the same transferrin or modified transferrin molecule. Such trans-bodies can bridge two different antigens or bind and/or activate two different cells. Thus, the present invention provides chimeric antibody variable regions fused to transferrin or modified transferrin. Moreover, the variable regions can be inserted into a transferrin or modified transferrin molecule.

The present invention contemplates trans-bodies that bind specifically to a desired polypeptide, peptide, or epitope. Trans-bodies are determined to be specifically binding if: 1) they exhibit a threshold level of binding activity, and/or 2) they do not significantly cross-react with unrelated polypeptide molecules. In some instances, trans-bodies specifically bind if they bind to a desired polypeptide, peptide or epitope with an affinity at least 10-fold greater than the binding affinity to control polypeptide. It is preferred that the trans-bodies exhibit a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of a trans-body of the invention can be readily determined by one of ordinary skill in the art using standard antibody affinity assays, for example, by Scatchard analysis (Scatchard, G., Ann. NY Acad. Sci. 51: 660-672, 1949).

In other embodiments, trans-bodies are determined to specifically bind if they do not significantly cross-react with unrelated polypeptides. Trans-bodies do not significantly cross-react with unrelated polypeptide molecules, for example, if they detect the desired polypeptide, peptide, or epitope but not unrelated polypeptides, peptides or epitopes, using a standard Western blot analysis. In some cases, unrelated polypeptides are orthologs, proteins from the same species that are members of a protein family.

Antibody Variable Regions for Generating Trans-Bodies

Variable regions from any number of antibodies may be converted to a form suitable for incorporation into transferrin for producing trans-bodies. These include anti-erbB2, B3, BR96, OVB3, anti-transferrin, Mik-β1 and PR1 (see Batra et al., Mol. Cell. Biol., 11: 2200-2205 (1991); Batra et al., Proc. Natl. Acad. Sci. USA, 89: 5867-5871 (1992); Brinkmann, et al. Proc. Natl. Acad. Sci. USA, 88: 8616-8620 (1991); Brinkmann et al., Proc. Natl. Acad. Sci. USA, 90: 547-551 (1993); Chaudhary et al., Proc. Natl. Acad. Sci. USA, 87: 1066-1070 (1990); Friedman et al., Cancer Res. 53: 334-339 (1993); Kreitman et al., J. Immunol., 149: 2810-2815 (1992); Nicholls et al., J. Biol. Chem., 268: 5302-5308 (1993); and Wells, et al., Cancer Res., 52: 6310-6317 (1992), respectively).

Typically, the Fv domains have been selected from the group of monoclonal antibodies known by their abbreviations in the literature as 26-10, MOPC 315, 741F8, 520C9, McPC 603, D1.3, murine phOx, human phOx, RFL3.8 sTCR, 1A6, Se155-4, 18-2-3, 4-4-20, 7A4-1, B6.2, CC49, 3C2, 2c, MA-15C5/K₁₂ G₀, Ox, etc. (see, Huston, J. S. et al., Proc. Natl. Acad. Sci. USA 85:5879-5883 (1988); Huston, J. S. et al., SIM News 38(4) (Supp.):11 (1988); McCartney, J. et al., ICSU Short Reports 10:114 (1990); Nedelman, M. A. et al., J. Nuclear Med. 32 (Supp.):1005 (1991); Huston, J. S. et al., In: Molecular Design and Modeling: Concepts and Applications, Part B, edited by J. J. Langone, Methods in Enzymology 203:46-88 (1991); Huston, J. S. et al., In: Advances in the Applications of Monoclonal Antibodies in Clinical Oncology, Epenetos, A. A. (Ed.), London, Chapman & Hall (1993); Bird, R. E. et al., Science 242:423-426 (1988); Bedzyk, W. D. et al., J. Biol. Chem. 265:18615-18620 (1990); Colcher, D. et al., J. Nat. Cancer Inst. 82:1191-1197 (1990); Gibbs, R. A. et al., Proc. Natl. Acad. Sci. USA 88:4001-4004 (1991); Milenic, D. E. et al., Cancer Research 51:6363-6371 (1991); Pantoliano, M. W. et al., Biochemistry 30:10117-10125 (1991); Chaudhary, V. K. et al., Nature 339:394-397 (1989); Chaudhary, V. K. et al., Proc. Natl. Acad. Sci. USA 87:1066-1070 (1990); Batra, J. K. et al., Biochem. Biophys. Res. Comm. 171:1-6 (1990); Batra, J. K. et al., J. Biol. Chem. 265:15198-15202 (1990); Chaudhary, V. K. et al., Proc. Natl. Acad. Sci. USA 87:9491-9494 (1990); Batra, J. K. et al., Mol. Cell. Biol. 11:2200-2205 (1991); Brinkmann, U. et al., Proc. Natl. Acad Sci. USA 88:8616-8620 (1991); Seetharam, S. et al., J. Biol. Chem. 266:17376-17381 (1991); Brinkmann, U. et al., Proc. Natl. Acad. Sci. USA 89:3075-3079 (1992); Glockshuber, R. et al., Biochemistry 29:1362-1367 (1990); Skerra, A. et al., Bio/Technol. 9:273-278 (1991); Pack, P. et al., Biochemistry 31:1579-1534 (1992); Clackson, T. et al., Nature 352:624-628 (1991); Marks, J. D. et al., J. Mol. Biol. 222:581-597 (1991); Iverson, B. L. et al., Science 249:659-662 (1990); Roberts, V. A. et al., Proc. Natl. Acad. Sci. USA 87:6654-6658 (1990); Condra, J. H. et al., J. Biol. Chem. 265:2292-2295 (1990); Laroche, Y. et al., J. Biol. Chem. 266:16343-16349 (1991); Holvoet, P. et al., J. Biol. Chem. 266:19717-19724 (1991); Anand, N. N. et al., J. Biol. Chem. 266:21874-21879 (1991); Fuchs, P. et al., Bio/Technol. 9:1369-1372 (1991); Breitling, F. et al., Gene 104:104-153 (1991); Seehaus, T. et al., Gene 114:235-237 (1992); Takkinen, K. et al., Protein Engng. 4:837-841 (1991); Dreher, M. L. et al., J. Immunol. Methods 139:197-205 (1991); Mottez, E. et al., Eur. J. Immunol. 21:467-471 (1991); Traunecker, A. et al., Proc. Natl. Acad. Sci. USA 88:8646-8650 (1991); Traunecker, A. et al., EMBO J. 10:3655-3659 (1991); Hoo, W. F. S. et al., Proc. Natl. Acad. Sci. USA 89:4759-4763 (1993)).

Table 1 provides various monoclonal antibodies whose variable regions and CDRs could be used to generate trans-bodies.

TABLE 1
Monoclonal Antibodies

| | | | | | |
|---|---|---|---|---|---|
| Category | Sub-Category | Drug Name | Brand | Indications | Target |

Inhibition of B and T-cell Activation

| | | | | | | |
|---|---|---|---|---|---|---|
| 1. | Immunology | Inhibition of B | BMS-188667 | | Arthritis, | CD-80 |
| | | and T-cell | | | rheumatoid | |
| | | Activation | | | Psoriasis | |
| | | | | | Transplant | |
| | | | | | rejection, bone | |
| | | | | | marrow | |
| 2. | Immunology | Inhibition of B | anti-B7 MAbs, | | Transplant | alpha- |
| | | and T-cell | Wyeth | | rejection, general | 4/beta-7 |
| | | Activation | | | Transplant | integrin |
| | | | | | rejection, bone | receptor |
| | | | | | marrow | |
| 3. | Immunology | Inhibition of B | BLyS | | Lupus | Blys |
| | | and T-cell | antagonists, | | erythematosus, | |
| | | Activation | CAT | | systemic | |
| | | | | | Arthritis, | |
| | | | | | rheumatoid | |
| 4. | Immunology | Inhibition of B | efalizumab | | Psoriasis | CD11alpha |
| | | and T-cell | | | Transplant | (alphaL |
| | | Activation | | | rejection, general | integrin) |
| | | | | | Arthritis, | |
| | | | | | rheumatoid | |
| 5. | Immunology | Inhibition of B | gavilimomab | | Transplant | CD147 |
| | | and T-cell | | | rejection, general | |
| | | Activation | | | Transplant | |
| | | | | | rejection, bone | |
| | | | | | marrow | |
| 6. | Immunology | Inhibition of B | siplizumab | | Transplant | T cells |
| | | and T-cell | | | rejection, bone | |
| | | Activation | | | marrow | |
| | | | | | Psoriasis, arthritis, | |
| | | | | | psoriatic | |
| 7. | Immunology | Inhibition of B | basiliximab | Simulect | Prophylaxis of | IL-2 |
| | | and T-cell | | | acute rejection in | Receptor or |
| | | Activation | | | kidney transplant | CD25 |
| | | | | | patients | antigen |
| 8. | Immunology | Inhibition of B | daclizumab | Zenapax | Transplant | alpha |
| | | and T-cell | | | rejection, general, | subunit of |
| | | Activation | | | Various cancer and | IL-2 |
| | | | | | autoimmune | |
| | | | | | diseases | |
| 9. | Immunology | Inhibition of B | OKT3A | Orthoclone | Transplant rejection | CD3 |
| | | and T-cell | | | | |
| | | Activation | | | | |
| 10. | Immunology | Inhibition of B | anti-CD3H | | Transplant | CD3H |
| | | and T-cell | | | rejection, general | |
| | | Activation | | | Ischaemia, cerebral | |
| | | | | | Reperfusion injury | |
| | | | | | Infarction, | |
| | | | | | myocardial | |
| | | | | | Inflammation, | |
| | | | | | general | |
| 11. | Immunology | Inhibition of B | muromonab- | | Transplant rejection | CD3 |
| | | and T-cell | CD3 | | | |
| | | Activation | | | | |
| 12. | Immunology | Inhibition of B | visilizumab | | Transplant | CD3 |
| | | and T-cell | | | rejection, bone | |
| | | Activation | | | marrow | |
| | | | | | Cancer, lymphoma, | |
| | | | | | T-cell, colitis, | |
| | | | | | ulcerative, | |
| | | | | | Myelodysplastic | |
| | | | | | syndrome | |
| | | | | | Lupus | |
| | | | | | erythematosus, | |
| | | | | | systematic | |
| 13. | Immunology | Inhibition of B | clenoliximab | | Arthritis, | CD4 |
| | | and T-cell | | | rheumatoid | |
| | | Activation | | | Asthma | |
| | | | | | Psoriasis | |
| 14. | Immunology | Inhibition of B | HuMax-CD4 | | Arthritis, | CD4 |
| | | and T-cell | | | rheumatoid | Receptor on T |
| | | Activation | | | Psoriasis | lymphocytes |
| 15. | Immunology | Inhibition of B | TNX-100 | | Crohn's disease | CD40 |
| | | and T-cell | | | | |
| | | Activation | | | | |
| 16. | Immunology | Inhibition of B | 5D12 | | Crohn's disease | CD40 |
| | | and T-cell | | | Psoriasis | |
| | | Activation | | | | |
| 17. | Immunology | Inhibition of B | HuMax-IL-15 | | Arthritis, | IL-15 |
| | | and T-cell | | | rheumatoid | |
| | | Activation | | | | |
| 18. | Immunology | Inhibition of B | inolimomab | | Transplant | IL-2 |
| | | and T-cell | | | rejection, bone | Receptor |
| | | Activation | | | marrow | |
| | | | | | Transplant | |
| | | | | | rejection, general | |
| 19. | Immunology | Inhibition of B | MRA, Chugai | | Arthritis, | IL-6 |
| | | and T-cell | | | rheumatoid | |
| | | Activation | | | Cancer, myeloma | |
| | | | | | Crohn's disease | |
| | | | | | Castleman's | |
| | | | | | disease | |
| | | | | | Arthritis, general | |
| 20. | Immunology | Inhibition of B | pascolizumab | | Asthma | IL-4 |
| | | and T-cell | | | | |
| | | Activation | | | | |
| 21. | Immunology | Inhibition of B | AGT-1 | | Arthritis, | alpha- |
| | | and T-cell | | | rheumatoid | interferon, |
| | | Activation | | | Multiple sclerosis, | gamma- |
| | | | | | general | interferon, |
| | | | | | | TNF |
| 22. | Immunology | Inhibition of B | afelimomab | | Sepsis | TNF-alpha |
| | | and T-cell | | | Transplant | |
| | | Activation | | | rejection, general | |
| 23. | Immunology | Inhibition of B | Humicade | | Crohn's disease | TNF |
| | | and T-cell | | | Arthritis, | |
| | | Activation | | | rheumatoid | |
| | | | | | Colitis, ulcerative | |
| | | | | | Diabetes, Type II | |
| 24. | Immunology | Inhibition of B | adalimumab | | Arthritis, | TNF |
| | | and T-cell | | | rheumatoid | |
| | | Activation | | | Crohn's disease | |
| 25. | Immunology | Inhibition of B | infliximab | Remicade | Crohn's disease | TNF-alpha |
| | | and T-cell | | | Arthritis, | |
| | | Activation | | | rheumatoid | |
| | | | | | Psoriasis | |
| 26. | Immunology | Inhibition of B | etanercept | Enbrel | Arthritis, | TNF |
| | | and T-cell | | | rheumatoid | |
| | | Activation | | | Psoriasis | |
| 27. | Immunology | Inhibition of B | CDP-870 | | Arthritis, | TNF-alpha |
| | | and T-cell | | | rheumatoid | |
| | | Activation | | | Crohn's disease | |

Inhibition of Complement Pathway

| | | | | | |
|---|---|---|---|---|---|
| 28. | Immunology | Inhibition of | pexelizumab | Infarction, | C5 |
| | | Complement | | myocardial | |
| | | Pathway | | Haemorrhage, | |
| | | | | general | |
| | | | | Ischaemia, cerebral | |
| 29. | Immunology | Inhibition of | eculizumab | Nephritis, general | C5 |
| | | Complement | | Arthritis, | complement |
| | | Pathway | | rheumatoid | inhibitor |
| | | | | Lupus nephritis | |
| | | | | Psoriasis | |
| | | | | Lupus | |
| | | | | erythematosus, | |
| | | | | systemic | |
| | | | | Inflammation, | |
| | | | | muscle | |
| | | | | Pemphigus | |
| | | | | Inflammation, | |
| | | | | dermal | |

Inhibition of Macrophage and Neutrophil Activation

| | | | | | |
|---|---|---|---|---|---|
| 30. | Immunology | Inhibition of | IDEC-114 | Psoriasis | CD80 |
| | | Macrophage | | Crohn's disease | |
| | | and Neutrophil | | | |
| | | Activation | | | |
| 31. | Other | Inhibition of | MDX-33 | Thrombocytopenic | FcR1 |
| | | Macrophage | | purpura | receptor |
| | | and Neutrophil | | Anaemia, general | |
| | | Activation | | | |
| 32. | Other | Inhibition of | SMART anti- | Unspecified | IL-12 |
| | | Macrophage | IL-12 MAb, | | |
| | | and Neutrophil | PDL | | |
| | | Activation | | | |
| 33. | Immunology | Inhibition of | J-695 | Arthritis, | IL-12 |
| | | Macrophage | | rheumatoid | |
| | | and Neutrophil | | | |
| | | Activation | | | |
| 34. | Immunology | Inhibition of | fontolizumab | Crohn's disease | IFN-gamma |
| | | Macrophage | | Psoriasis | |
| | | and Neutrophil | | | |
| | | Activation | | | |

Eosinophil and/or IgE Pathway

| | | | | | |
|---|---|---|---|---|---|
| 35. | Immunology | Eosinophil | IDEC-152 | Asthma | CD23 |
| | | and/or IgE | | | |
| | | Pathway | | | |
| 36. | Immunology | Eosinophil | CAT-213 | Rhinitis, allergic | eotaxin |
| | | and/or IgE | | | |
| | | Pathway | | | |
| 37. | Immunology | Eosinophil | E-26 | Asthma | bcl-2 |
| | | and/or IgE | | Rhinitis, allergic | |
| | | Pathway | | | |
| 38. | Immunology | Eosinophil | reslizumab | Asthma | IL-5 |
| | | and/or IgE | | Allergy, general | |
| | | Pathway | | Inflammation, | |
| | | | | general | |
| 39. | Immunology | Eosinophil | mepolizumab | Asthma | IL-5 |
| | | and/or IgE | | | |
| | | Pathway | | | |

Modulation of Vascular Adhesion of Inflammatory Cells

| | | | | | |
|---|---|---|---|---|---|
| 40. | Immunology | Modulation of | MLN-02 | Crohn's disease | alpha- |
| | | Vascular | | Colitis, ulcerative | 4/beta-7 |
| | | Adhesion of | | | integrin |
| | | Inflammatory | | | receptor |
| | | Cells | | | |
| 41. | Immunology | Modulation of | MLN-01 | Transplant | integrins |
| | | Vascular | | rejection, general | |
| | | Adhesion of | | Ischaemia, cerebral | |
| | | Inflammatory | | | |
| | | Cells | | | |
| 42. | Immunology | Modulation of | HuDREG-55 | Traumatic shock | block human |
| | | Vascular | MAb, PDL | | L-selectin |
| | | Adhesion of | | | adhesion of |
| | | Inflammatory | | | lymphocytes |
| | | Cells | | | |
| 43. | Immunology | Modulation of | humanized | Inflammation, | Human |
| | | Vascular | VAP-1 MAb, | general | vascular |
| | | Adhesion of | BioTie | | indothelium |
| | | Inflammatory | | | antigen |
| | | Cells | | | VAP-1 |
| 44. | Immunology | Modulation of | vepalimomab | Psoriasis | VAP-1 |
| | | Vascular | | Eczema, allergic, | murine Mab |
| | | Adhesion of | | general | |
| | | Inflammatory | | Colitis, ulcerative | |
| | | Cells | | Reperfusion injury | |
| | | | | Ischaemia, cerebral | |
| | | | | Respiratory distress | |
| | | | | syndrome, adult | |
| 45. | Immunology | Modulation of | natalizumab | Multiple sclerosis, | alpha-4 |
| | | Vascular | | relapsing-remitting | integrin |
| | | Adhesion of | | Crohn's disease | |
| | | Inflammatory | | Colitis, ulcerative | |
| | | Cells | | Arthritis, | |
| | | | | rheumatoid | |

HIV

| | | | | | |
|---|---|---|---|---|---|
| 46. | Infection | HIV | TNX-355 | Infection, HIV | CD4 |
| | | | | prophylaxis | |
| 47. | Infection | HIV | Cytolin | Infection, HIV/AIDS | LFH-1 |

Respiratory Infection

| | | | | | |
|---|---|---|---|---|---|
| 48. | Infection | Respiratory | IC-14 | Sepsis | treatment of |
| | | Infection | | Infection | sepsis |
| | | | | respiratory tract, | |
| | | | | lower | |

Hepatitis

| | | | | | |
|---|---|---|---|---|---|
| 49. | Infection | Hepatitis | XTL-002 | Infection, hepatitis- | HCV |
| | | | | C virus | |
| 50. | Infection | Hepatitis | XTL-001 | Infection, hepatitis- | HBV |
| | | | | B virus | |

Misc. Infections Diseases

| | | | | | | |
|---|---|---|---|---|---|---|
| 51. | Infection | Misc. Infections | E. coli O157 | | Infection, GI tract | E. coli O157 |
| | | Diseases | anti-verotoxin | | | |
| | | | MAb | | | |
| 52. | Infection | Misc. Infections | palivizumab | Synagis | Immunomodulator, | RSV |
| | | Diseases | | | anti-infective, for | |
| | | | | | treatment and | |
| | | | | | prevention of RSV | |
| | | | | | pneumonia in | |
| | | | | | infants | |
| 53. | Infection | Misc. Infections | hsp90 MAb, | | Infection | hsp90 |
| | | Diseases | NeuTec | | Candida, general | |
| 54. | Infection | Misc. Infections | BSYX-A110 | | Infection, | Lipoteichoic- |
| | | Diseases | | | staphylococcal | acid |
| | | | | | prophylaxis | |
| 55. | Infection | Misc. Infections | anti-MRSA | | Infection, MRSA | MRSA |
| | | Diseases | MAb, NeuTec | | | |

Growth Factor Receptors/Hormone Receptors

| | | | | | | |
|---|---|---|---|---|---|---|
| 56. | Cancer | Growth Factor | EMD-72000 | | Cancer, stomach | EGFR |
| | | Receptors/Hormone | | | Cancer, cervical | |
| | | Receptors | | | Cancer, lung, non- | |
| | | | | | small cell | |
| | | | | | Cancer, head and | |
| | | | | | neck | |
| | | | | | Cancer, ovarian | |
| 57. | Cancer | Growth Factor | R3 | | Cancer, head and | EGFR |
| | | Receptors/Hormone | | | neck | |
| | | Receptors | | | Diagnosis, cancer | |
| 58. | Cancer | Growth Factor | cetuximab | | Cancer, head and | EGFR |
| | | Receptors/Hormone | | | neck | |
| | | Receptors | | | Cancer, lung, non- | |
| | | | | | small cell | |
| | | | | | Cancer, colorectal | |
| | | | | | Cancer, breast | |
| | | | | | Cancer, pancreatic | |
| | | | | | Cancer, prostate | |
| 59. | Cancer | Growth Factor | ABX-EGF | | Cancer, renal | EGFR |
| | | Receptors/Hormone | | | Cancer, lung, non- | |
| | | Receptors | | | small cell | |
| | | | | | Cancer, colorectal | |
| | | | | | Cancer, prostate | |
| | | | | | Cancer, pancreatic | |
| | | | | | Cancer, | |
| | | | | | oesophageal | |
| 60. | Cancer | Growth Factor | trastuzumab | Herceptin | Breast cancer | HER2 |
| | | Receptors/Hormone | | | | |
| | | Receptors | | | | |
| 61. | Cancer | Growth Factor | 2C4 antibody, | | Cancer, breast | HER2 |
| | | Receptors/Hormone | Genentech | | | |
| | | Receptors | | | | |
| 62. | Cancer | Growth Factor | MDX-210 | | Cancer, ovarian | HER2 |
| | | Receptors/Hormone | | | Cancer, prostate | |
| | | Receptors | | | Cancer, colorectal | |
| | | | | | Cancer, renal | |
| | | | | | Cancer, breast | |

Hematological Tumor Markers

| | | | | | | |
|---|---|---|---|---|---|---|
| 63. | Cancer | Hematological | MT-103 | | Cancer, lymphoma, | CD19 |
| | | Tumor Markers | | | B-cell | |
| | | | | | Cancer, lymphoma, | |
| | | | | | non-Hodgkin's | |
| | | | | | Cancer, leukaemia, | |
| | | | | | chronic | |
| | | | | | myelogenous | |
| | | | | | Cancer, leukaemia, | |
| | | | | | acute myelogenous | |
| 64. | Cancer | Hematological | rituximab | Rituxan | Non-hodgkin's | CD20 |
| | | Tumor Markers | | | lymphoma | |
| 65. | Cancer | Hematological | SGN-30 | | Cancer, lymphoma, | CD30 |
| | | Tumor Markers | | | Hodgkin's | |
| | | | | | Cancer, lymphoma, | |
| | | | | | general | |
| 66. | Cancer | Hematological | H22xKi-4 | | Cancer, lymphoma, | CD64 and |
| | | Tumor Markers | | | Hodgkin's | CD30 |
| 67. | Cancer | Hematological | alemtuzumab | Campath | CLL | CD52 |
| | | Tumor Markers | | | | |
| 68. | Cancer | Hematological | ior-t1 | | Cancer, lymphoma, | CD6 |
| | | Tumor Markers | | | T-cell | |
| | | | | | Psoriasis | |
| | | | | | Arthritis, | |
| | | | | | rheumatoid | |
| 69. | Cancer | Hematological | apolizumab | | Cancer, lymphoma, | HLA-DR |
| | | Tumor Markers | | | non-Hodgkin's | |
| | | | | | Cancer, leukaemia, | |
| | | | | | chronic lymphocytic | |
| | | | | | Cancer, general | |
| 70. | Cancer | Hematological | anti-HMI.24 | | Cancer, myeloma | HMI.24 |
| | | Tumor Markers | antibody, | | | antigen |
| | | | Chugai | | | |

Apoptosis

| | | | | | |
|---|---|---|---|---|---|
| 71. | Cancer | Apoptosis | antiangiogenesis | Cancer, sarcoma, | angiogenesis |
| | | | MAb, AME | leiomyo | |
| | | | | Diagnosis, cancer | |
| | | | | Cancer, colorectal | |
| | | | | Arthritis, | |
| | | | | rheumatoid | |
| | | | | Arthritis, psoriatic | |
| 72. | Cancer | Apoptosis | Onyvax-105 | Cancer, colorectal | CD55 |
| | | | | Cancer, sarcoma, | |
| | | | | general | |
| 73. | Cancer | Apoptosis | TRAIL-R1 | Cancer, general | TRAIL |
| | | | MAb, CAT | | Receptor |

Epithelial Tumor Markers

| | | | | | |
|---|---|---|---|---|---|
| 74. | Cancer | Epithelial | MDX-220 | Cancer, prostate | TAG-72 |
| | | Tumor Markers | | Cancer, colorectal | |
| 75. | Cancer | Epithelial | KSB-303 | Diagnosis, cancer | CEA |
| | | Tumor Markers | | Cancer, colorectal | |
| | | | | Cancer, pancreatic | |
| 76. | Cancer | Epithelial | CeaVac | Cancer, colorectal | CEA |
| | | Tumor Markers | | Cancer, lung, non- | |
| | | | | small cell | |
| | | | | Cancer, breast | |
| | | | | Cancer, liver | |
| 77. | Cancer | Epithelial | MT-201 | Cancer, prostate | Ep-CAM |
| | | Tumor Markers | | Cancer, colorectal | |
| | | | | Cancer, stomach | |
| | | | | Cancer, lung, non- | |
| | | | | small cell | |
| 78. | Cancer | Epithelial | ING-1 | Cancer, breast | Ep-CAM |
| | | Tumor Markers | | Cancer, lung, | |
| | | | | general | |
| | | | | Cancer, ovarian | |
| | | | | Cancer, prostate | |
| 79. | Cancer | Epithelial | IGN-101 | Cancer, lung, non- | Ep-CAM |
| | | Tumor Markers | | small cell | |
| | | | | Cancer, liver | |
| | | | | Cancer, colorectal | |
| | | | | Cancer, | |
| | | | | oesophageal | |
| | | | | Cancer, stomach | |
| 80. | Cancer | Epithelial | BrevaRex | Cancer, myeloma | MUC1 |
| | | Tumor Markers | MAb | Cancer, breast | antigen |
| 81. | Cancer | Epithelial | Imuteran | Cancer, breast | |
| | | Tumor Markers | | Cancer, ovarian | |
| 82. | Cancer | Epithelial | ABX-MA1 | Cancer, melanoma | MUC1 |
| | | Tumor Markers | | | antigen |
| 83. | Cancer | Epithelial | Therex | Cancer, breast | MUC1 |
| | | Tumor Markers | | | |

Anti-angiogenesis

| | | | | | |
|---|---|---|---|---|---|
| 84. | Cancer | Anti- | bevacizumab | Cancer, colorectal | VEGF |
| | | angiogenesis | | Cancer, breast | |
| | | | | Cancer, lung, non- | |
| | | | | small cell | |
| | | | | Cancer, renal | |
| | | | | Retinopathy, | |
| | | | | diabetic | |

Misc. Tumor Markers

| | | | | | | |
|---|---|---|---|---|---|---|
| 85. | Cancer | Misc. Tumor | CDP-860 | | Cancer, general | PDGF-Beta |
| | | Markers | | | Restenosis | receptor |
| 86. | Cancer | Misc. Tumor | oregovomab | | Cancer, ovarian | CA125 |
| | | Markers | | | | |
| 87. | Cancer | Misc. Tumor | MDX-010 | | Cancer, prostate | CTLA-4 |
| | | Markers | | | Cancer, melanoma | |
| | | | | | Infection, general | |
| 88. | Cancer | Misc. Tumor | ecromeximab | | Cancer, melanoma | GD3 |
| | | Markers | | | | ganglioside |
| 89. | Cancer | Misc. Tumor | huJ591 MAb, | | Cancer, prostate | PSMA |
| | | Markers | BZL | | Cancer, general | |
| 90. | Cancer | Misc. Tumor | anti-PTHrP | | Hypercalcaemia of | PTHrP |
| | | Markers | antibody, | | malignancy | |
| | | | Chugai | | Cancer, bone | |
| 91. | Cancer | Misc. Tumor | AR54 | | Cancer, ovarian | TAG72 |
| | | Markers | | | | |
| 92. | Cancer | Misc. Tumor | Pharmaprojects | | Cancer, general | |
| | | Markers | No. 5876 | | | |
| 93. | Cancer | Misc. Tumor | prostate | | Cancer, prostate | Prostate |
| | | Markers | cancer Ab, | | | Cancer Cells |
| | | | Biovation | | | |
| 94. | Cancer | Misc. Tumor | VB2-011 | | Cancer, lymphoma, | anticancer |
| | | Markers | | | non-Hodgkin's | effect |
| | | | | | Cancer, melanoma | human |
| | | | | | | breast tumor |
| | | | | | | model |
| 95. | Cancer | Misc. Tumor | TriAb | | Cancer, breast | HMFG |
| | | Markers | | | Cancer, lung, non- | |
| | | | | | small cell | |
| | | | | | Cancer, colorectal | |
| 96. | Cancer | Misc. Tumor | TriGem | | Cancer, melanoma | GD2 |
| | | Markers | | | Cancer, lung, small | ganglioside |
| | | | | | cell | |
| | | | | | Cancer, brain | |
| 97. | Cancer | Misc. Tumor | G-250, | | Cancer, renal | RCC |
| | | Markers | unconjugated | | | |
| 98. | Cancer | Misc. Tumor | ACA-125 | | Cancer, ovarian | CA125 |
| | | Markers | | | | |
| 99. | Cancer | Misc. Tumor | mitumomab | | Cancer, lung, small | GD3 |
| | | Markers | | | cell | ganglioside |
| | | | | | Cancer, melanoma | |
| 100. | Cancer | Misc. Tumor | edrecolomab | | Cancer, colorectal | Ep-CAM |
| | | Markers | | | Cancer, breast | |
| 101. | Other | | SB-249417 | | Sepsis | Factor IX |
| | | | | | Ischaemia, cerebral | |
| 102. | Other | | YM-337 | | Surgery adjunct | GPIIb/IIIa |
| | | | | | Ischaemic | |
| | | | | | cardiomyopathy | |
| | | | | | Thrombosis, | |
| | | | | | general | |
| | | | | | Transplant | |
| | | | | | rejection, general | |
| | | | | | Angina, unstable | |
| | | | | | Ischaemia, cerebral | |
| 103. | Other | | abciximab | ReoPro | clot-related | GPIIb/IIIa |
| | | | | | cardiovascular | |
| | | | | | disease (high risk | |
| | | | | | angioplasty), | |
| | | | | | complication of | |
| | | | | | coronary | |
| | | | | | angioplasty | |
| 104. | Other | | TNX-901 | | Allergy, food | IgI |
| 105. | Other | | CAT-192 | | Scleroderma | TGF-beta1 |
| 106. | Other | | lerdelimumab | | Fibrosis, general | TGF-beta2 |
| | | | | | Surgery adjunct | |
| 107. | Other | | Pharmaprojects | | Unspecified | |
| | | | No. 6256 | | | |
| 108. | Other | | rhuFabV2 | | Macular | VEGF |
| | | | | | degeneration | |
| 109. | Other | | RN-2 | | Wound healing | |
| 110. | Other | | heteropolymer | | Unspecified | anthrax toxin |
| | | | technol, | | | |
| | | | EluSys | | | |
| 111. | Other | | PRIMATIZED | | Unspecified | CD23 |
| | | | antibodies, | | | |
| | | | IDEC | | | |
| 112. | Other | | DBI-5012 | | Diabetes, Type I | |
| 113. | Payload | | ibritumomab | Zevalin | non-hodgkin's | CD20 |
| | | | tiuxetan | | lymphoma | |
| 114. | Payload | | epratuzumab | | Cancer, lymphoma, | CD22 |
| | | | | | non-Hodgkin's | |
| 115. | Payload | | gemtuzumab | Mylotarg | AML (acute myeloid | CD33 |
| | | | ozogamicin | | leukemia) | |
| 116. | Payload | | labetuzumab | | Cancer, breast | CEA |
| | | | | | Cancer, lung, small | |
| | | | | | cell | |
| | | | | | Cancer, ovarian | |

Humanized Antibody Variable Region

The present invention also contemplates the production and use of humanized variable domains for making trans-bodies. Humanized antibodies are non human antibodies in which some or all of the amino acid residues are replaced with the corresponding amino acid residue found in a similar human antibody. Typically, residues in the hypervariable region and possibly in the FR are substituted by residues from analogous sites in rodent antibodies. Humanization reduces the antigenic potential of the antibody.

Antibody variable domains have been humanized by various methods, such as CDR grafting (Riechmann et al., Nature, 332: 323-327 (1988)), replacement of exposed residues (Padlan, Mol. Immunol. 28: 489-498 (1991)) and variable domain resurfacing (Roguska et al., Proc. Natl. Acad. Sci. USA, 91: 969-973 (1994). The minimalistic approach of resurfacing is particularly suitable for antibody variable domains which require preservation of some mouse, or other species', framework residues to maintain maximal antigen binding affinity. However, CDR grafting approach has also been successfully used for the humanization of several antibodies either without preserving any of the mouse framework residues (Jones et al. Nature, 321: 522-525 (1986) and Verhoeyen et al., Science, 239: 1534-1536 (1988)) or with the preservation of just one or two mouse residues (Riechmann et al., Nature, 332: 323-327 (1988); Queen et al., Proc. Natl. Acad. Sci. USA, 86: 10029-10033 (1989).

Humanization can also be accomplished by aligning the variable domains of the heavy and light chains with the best human homolog identified in sequence databases such as GENBANK or SWISS-PROT using standard sequence comparison software. Sequence analysis and comparison to a structural model based on the crystal structure of the variable domains of monoclonal antibody McPC603 (Queen et al., Proc. Natl. Acad. Sci. USA, 86: 10029-10033 (1989) and Satow et al., J. Mol. Biol. 190: 593-604 (1986)); Protein Data bank Entry IMCP) allows identification of the framework residues that differ between the mouse antibody and its human counterpart.

The choice of human variable domains, both light and heavy, to be used in making the humanized antibodies is important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup. of light or heavy chains. The same framework may be used for several-different humanized antibodies (Carter et al., Proc. Natl. Acad. Sci. USA, 89:4285 (1992); Presta et al., J. Immnol., 151:2623 (1993)).

Production of Antigen Binding Fragments and CDRs

Antigen binding fragments and CDRs that may be fused or attached to transferrin may be produced by several methods including but not limited to: selection from phage libraries, cloning of the variable region of a specific antibody by cloning the cDNA of the antibody and using the flanking constant regions as the primer to clone the variable region, or by synthesizing an oligonucleotide corresponding to the variable region of any specific antibody. The cDNA can be tailored at the 5′ and 3′ ends to generate restriction sites, such that oligonucleotide linkers can be used, for cloning of the cDNA into a vector containing the cDNA for transferrin. This can be at the N- or C-terminus or N- and C-termini with or without the use of a spacer sequence. The fusion molecule cDNA may be cloned into a vector from which the complete expression cassette is then excised and inserted into an expression vector to allow the expression of the fusion protein in yeast. The fusion protein secreted from the yeast can then be collected and purified from the media and tested for its activity. For expression in mammalian cell lines a similar procedure is adopted except that the expression cassette used employs a mammalian promoter, leader sequence and terminator. This expression cassette is then excised and inserted into a plasmid suitable for the transfection of mammalian cell lines. The trans-body produced in this manner can be purified from media and tested for its binding to its antigen using standard immunochemical methods.

In particular, phage display technology may be used to generate large libraries of antigen binding peptides by exploiting the capability of bacteriophage to express and display biologically functional protein molecule on its surface. In other embodiments, the library of antigen binding peptides may be prepared directly in modified Tf to create a trans-body library. Combinatorial libraries of antigen binding peptides have been generated in bacteriophage lambda expression systems which may be screened as bacteriophage plaques or as colonies of lysogens (Huse et al. (1989) Science 246: 1275; Caton and Koprowski (1990) Proc. Natl. Acad. Sci. (U.S.A.) 87: 6450; Mullinax et al. (1990) Proc. Natl. Acad. Sci. (U.S.A.) 87: 8095; Persson et al. (1991) Proc. Natl. Acad. Sci. (U.S.A.) 88: 2432). Various embodiments of bacteriophage antigen binding peptides display libraries and lambda phage expression libraries have been described (Kang et al. (1991) Proc. Natl. Acad. Sci. (U.S.A.) 88: 4363; Clackson et al. (1991) Nature 352: 624; McCafferty et al. (1990) Nature 348: 552; Burton et al. (1991) Proc. Natl. Acad. Sci. (U.S.A.) 88: 10134; Hoogenboom et al. (1991) Nucleic Acids Res. 19: 4133; Chang et al. (1991) J. Immunol. 147: 3610; Breitling et al. (1991) Gene 104: 147; Marks et al. (1991) J. Mol. Biol. 222: 581; Barbas et al. (1992) Proc. Natl. Acad. Sci. (U.S.A.) 89: 4457; Hawkins and Winter (1992) J. Immunol. 22: 867; Marks et al. (1992) Biotechnology 10: 779; Marks et al. (1992) J. Biol. Chem. 267: 16007; Lowman et al. (1991) Biochemistry 30: 10832; Lemer et al. (1992) Science 258: 1313). Also see review by Rader, C. and Barbas, C. F. (1997) “Phage display of combinatorial antibody libraries” Curr. Opin. Biotechnol. 8:503-508. Various scFv libraries displayed on bacteriophage coat proteins have been described (Marks et al. (1992) Biotechnology 10: 779; Winter G and Milstein C (1991) Nature 349: 293; Clackson et al. (1991) op.cit.; Marks et al. (1991) J. Mol. Biol. 222: 581; Chaudhary et al. (1990) Proc. Natl. Acad. Sci. (USA) 87: 1066; Chiswell et al. (1992) TIBTECH 10: 80; and Huston et al. (1988) Proc. Natl. Acad. Sci. (USA) 85: 5879).

Generally, a phage library is created by inserting a library of a random oligonucleotide or a cDNA library encoding antibody fragment or peptide such as V_{L} and V_{H} into gene 3 of M13 or fd phage. Each inserted gene is expressed at the N-terminal of the gene 3 product, a minor coat protein of the phage. As a result, peptide libraries that contain diverse peptides can be constructed. The phage library is then affinity screened against immobilized target molecule of interest, such as an antigen, and specifically bound phages are recovered and amplified by infection into Escherichia coli host cells. Typically, the target molecule of interest such as a receptor (e.g., polypeptide, carbohydrate, glycoprotein, nucleic acid) is immobilized by covalent linkage to a chromatography resin to enrich for reactive phage by affinity chromatography) and/or labeled for screen plaques or colony lifts. Finally, amplified phages can be sequenced for deduction of the specific peptide sequences. Due to the inherent nature of phage display, the antibodies or peptides displayed on the surface of the phage may not adopt its native conformation under such in vitro selection conditions as in a mammalian system. In addition, bacteria do not readily process, assemble, or express/secrete functional antibodies.

As part of this invention, transferrin or part of transferrin containing random peptides can be inserted into gene 3 of the phage instead of V_{L} or V_{H} fragments. In this manner the library can be screened for a transferrin protein which contains an antigenic peptide.

Transgenic animals such as mice have been used to generate fully human antibodies by using the XENOMOUSE™ technology developed by companies such as Abgenix, Inc., Fremont, Calif. and Medarex, Inc. Annandale, N.J. Strains of mice are engineered by suppressing mouse antibody gene expression and functionally replacing it with human antibody gene expression. This technology utilizes the natural power of the mouse immune system in surveillance and affinity maturation to produce a broad repertoire of high affinity antibodies.

In yet another aspect of the present invention, the method for producing a library of single chain antibodies comprises: expressing in yeast cells a library of yeast expression vectors. Each of the yeast expression vectors comprises a first nucleotide sequence encoding an antibody heavy chain variable region, a second nucleotide sequence encoding an antibody light chain variable region, and a transferrin sequence that links the antibody heavy chain variable region and the antibody light chain variable region. The antibody heavy chain variable region, the antibody light chain variable region, and the transferrin linker are expressed as a single trans-body fusion protein. Also, the first and second nucleotide sequences each independently varies within the library of expression vectors to generate a library of trans-bodies with a diversity of at least about 10⁶.

In a similar manner, a library can express transferrin containing various inserted peptides instead of antibody fragments. This library is then screened for the trans-body with the best binding activity for a particular antigen.

According to the embodiment, the diversity of the library of trans-bodies is preferably between about 10⁶-10¹⁶, more preferably between about 10⁸-10¹⁶, and most preferably between about 10¹⁰-10¹⁶.

Therapeutic Trans-Bodies

The present invention also involves making and using trans-bodies comprising antibody variable regions from antibodies directed against one or more different antigens for the treatment or prevention of diseases. Preferably, at least one of the antigens (and preferably all of the antigens are) is a biologically important molecule and administration of a trans-body against the antigen to a mammal suffering from a disease or disorder can result in a therapeutic benefit in that mammal. In the preferred embodiment of the invention, the antigen is a protein. However, other nonpolypeptide antigens (e.g. tumor associated glycolipids; see U.S. Pat. No. 5,091,178) may be used.

Exemplary protein antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or-tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-.beta.; platelet-derived growth factor (PDGF); fibroblast growth factor such as aFGF and bFGF; epidermal growth factor (EGF); transforming growth factor (TGF) such as TGF-alpha and TGF-beta, including TGF-.beta. 1, TGF-.beta.2, TGF-.beta.3, TGF-.beta.4, or TGF-.beta.5; insulin-like growth factor-I and -II (IGF-I and IGF-II); des(1-3)-IGF-I (brain IGF-I), insulin-like growth factor binding proteins; CD proteins such as CD3, CD4, CD8, CD19 and CD20; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; RSV envelop protein; HSV envelop and coat proteins; influenza virus coat protein; integrins such as CD11a, CD11b, CD11c, CD18, an ICAM, VLA-4 and VCAM; a tumor associated antigen such as HER2, HER3 or HER4 receptor; bacteria and their toxins such as botulinum toxin, cholera toxin, and anthrax toxin; fungi, specifically pathogenic fungi; and variants and/or fragments of any of the above-listed polypeptides. Additional molecules to which trans-bodies of the invention may bind are listed in PCT/US02/27637, which is herein incorporated by reference in its entirety.

Transferrin and Transferrin Modifications

The present invention provides trans-bodies comprising one or more antibody variable regions and transferrin or modified transferrin. Any transferrin may be used to make modified Tf fusion proteins of the invention.

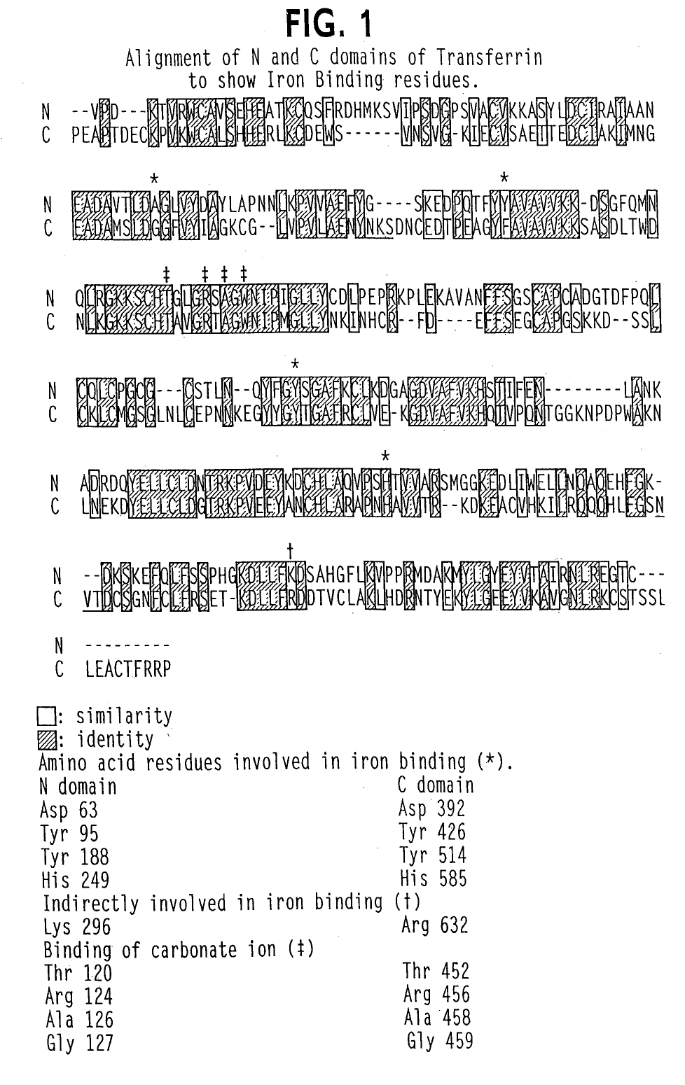

Wild-type human Tf (Tf) is a 679 amino acid protein, of approximately 75 kDa (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and S95936 (www.ncbi.nlm.nih.gov), all of which are herein incorporated by reference in their entirety, as well as SEQ ID NOS: 1, 2 and 3. The two domains have diverged over time but retain a large degree of identity/similarity (FIG. 1).

Each of the N and C domains is further divided into two subdomains, N1 and N2, C1 and C2. The function of Tf is to transport iron to the cells of the body. This process is mediated by the Tf receptor (TfR), which is expressed on all cells, particularly actively growing cells. TfR recognizes the iron bound form of Tf (two of which are bound per receptor), endocytosis then occurs whereby the TfR/Tf complex is transported to the endosome, at which point the localized drop in pH results in release of bound iron and the recycling of the TfR/Tf complex to the cell surface and release of Tf (known as apoTf in its un-iron bound form). Receptor binding is through the C domain of Tf. The two glycosylation sites in the C domain do not appear to be involved in receptor binding as unglycosylated iron bound Tf does bind the receptor.

Each Tf molecule can carry two iron atoms. These are complexed in the space between the N1 and N2, C1 and C2 subdomains resulting in a conformational change in the molecule. Tf crosses the blood brain barrier (BBB) via the Tf receptor.

In human transferrin, the iron binding sites comprise at least of amino acids Asp 63 (Asp 82 of SEQ ID NO: 2 which comprises the native Tf signal sequence); Asp 392 (Asp 411 of SEQ ID NO: 2); Tyr 95 (Tyr 114 of SEQ ID NO: 2); Tyr 426 (Tyr 445 of SEQ ID NO: 2); Tyr 188 (Tyr 207 of SEQ ID NO: 2); Tyr 514 or 517 (Tyr 533 or Tyr 536 SEQ ID NO:2); His 249 (His 268 of SEQ ID NO: 2); His 585 (His 604 of SEQ ID NO: 2), the hinge regions comprise at least N domain amino acid residues 94-96, 245-247 and/or 316-318 as well as C domain amino acid residues 425-427, 581-582 and/or 652-658., the carbonate binding sites comprise at least of amino acids Thr 120 (Thr 139 of SEQ ID NO: 2); Thr 452 (Thr 471 of SEQ ID NO: 2); Arg 124 (Arg 143 of SEQ ID NO: 2); Arg 456 (Arg 475 of SEQ ID NO: 2); Ala 126 (Ala 145 of SEQ ID NO: 2); Ala 458 (Ala 477 of SEQ ID NO: 2); Gly 127 (Gly 146 of SEQ ID NO: 2); Gly 459 (Gly 478 of SEQ ID NO: 2).

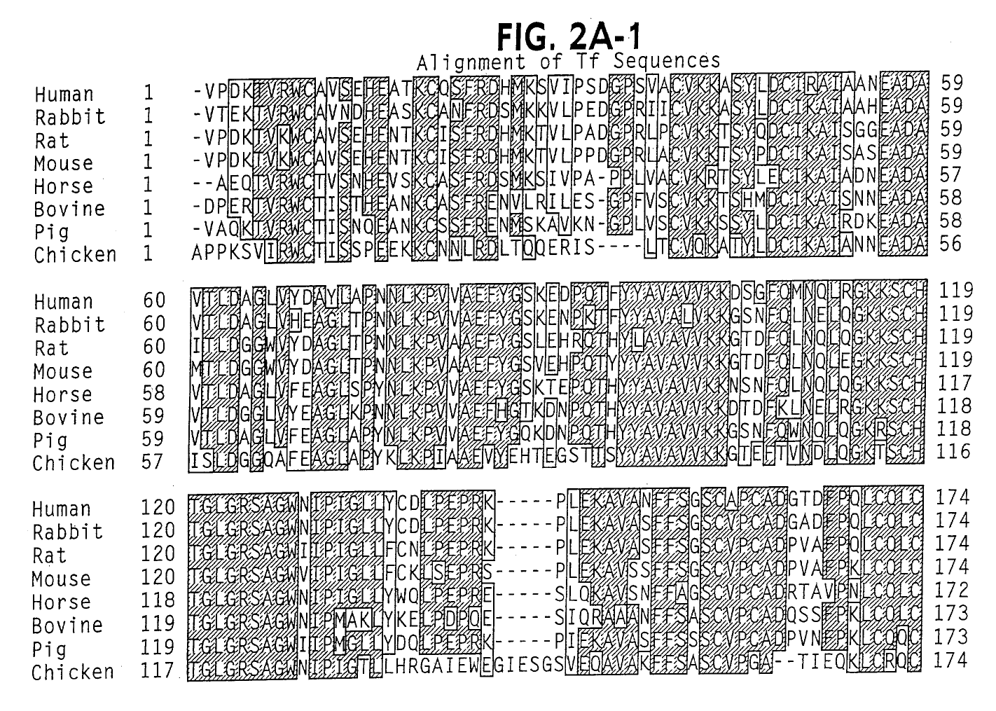

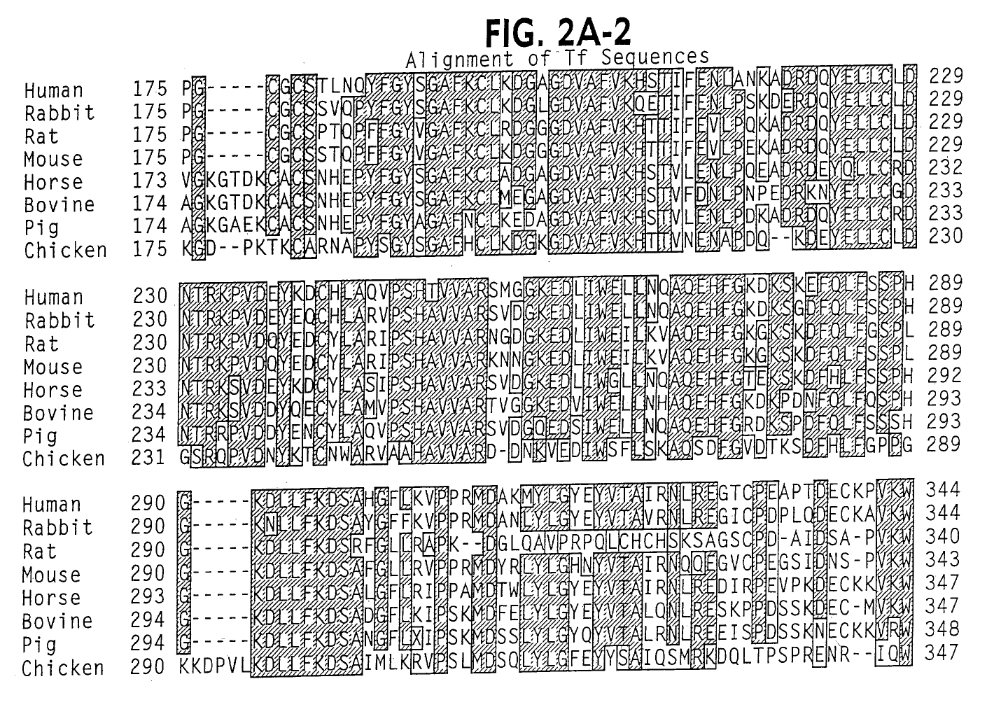

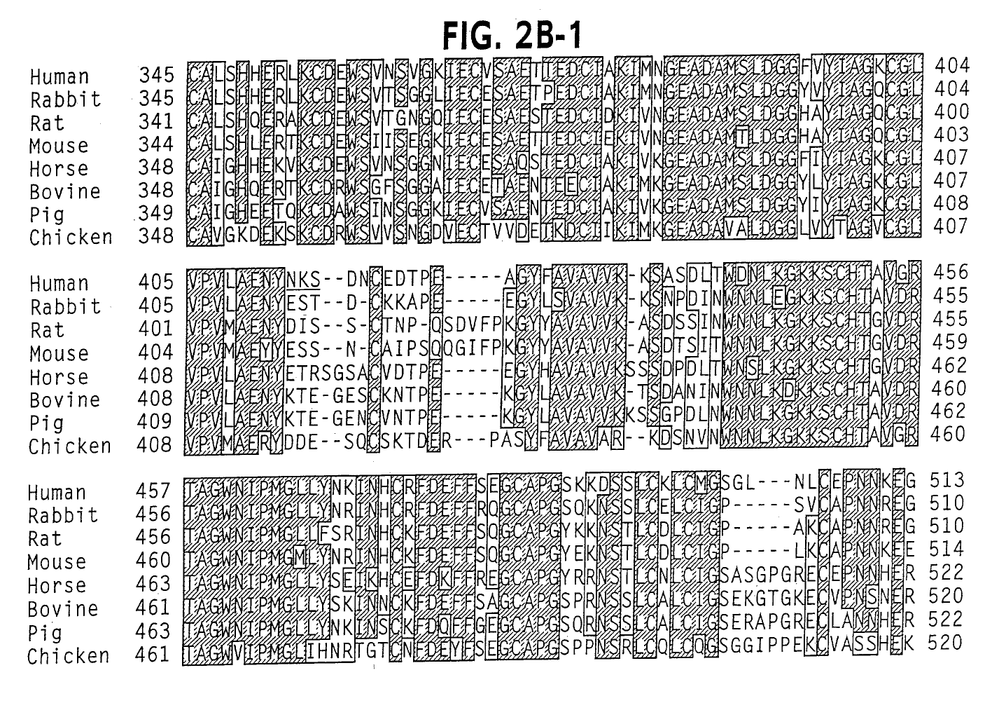

In one embodiment of the invention, the trans-body includes a modified human transferrin, although any animal Tf molecule may be used to produce the trans-bodies of the invention, including human Tf variants, cow, pig, sheep, dog, rabbit, rat, mouse, hamster, echnida, platypus, chicken, frog, hornworm, monkey, as well as other bovine, canine and avian species (see FIG. 2 for a representative set of Tf sequences). All of these Tf sequences are readily available in GenBank and other public databases. The human Tf nucleotide sequence is available (see SEQ ID NOS: 1, 2 and 3 and the accession numbers described above and available at www.ncbi.nlm.nih.gov/) and can be used to make genetic fusions between Tf or a domain of Tf and the therapeutic molecule of choice. Fusions may also be made from related molecules such as lacto transferrin (lactoferrin) GenBank Acc: NM_{—}002343).

Lactoferrin (Lf), a natural defense iron-binding protein, has been found to possess antibacterial, antimycotic, antiviral, antineoplastic and anti-inflammatory activity. The protein is present in exocrine secretions that are commonly exposed to normal flora: milk, tears, nasal exudate, saliva, bronchial mucus, gastrointestinal fluids, cervico-vaginal mucus and seminal fluid. Additionally, Lf is a major constituent of the secondary specific granules of circulating polymorphonuclear neutrophils (PMNs). The apoprotein is released on degranulation of the PMNs in septic areas. A principal function of Lf is that of scavenging free iron in fluids and inflamed areas so as to suppress free radical-mediated damage and decrease the availability of the metal to invading microbial and neoplastic cells. In a study that examined the turnover rate of ¹²⁵I Lf in adults, it was shown that LF is rapidly taken up by the liver and spleen, and the radioactivity persisted for several weeks in the liver and spleen (Bennett et al. (1979), Clin. Sci. (Lond.) 57: 453-460).

In one embodiment, the transferrin portion of the trans-body of the invention includes a transferrin splice variant. In one example, a transferrin splice variant can be a splice variant of human transferrin. In one specific embodiment, the human transferrin splice variant can be that of Genbank Accession AAA61140.

In another embodiment, the transferrin portion of the trans-body of the invention includes a lactoferrin splice variant. In one example, a human serum lactoferrin splice variant can be a novel splice variant of a neutrophil lactoferrin. In one specific embodiment, the neutrophil lactoferrin splice variant can be that of Genbank Accession AAA59479. In another specific embodiment, the neutrophil lactoferrin splice variant can comprise the following amino acid sequence EDCIALKGEADA (SEQ ID NO: 4), which includes the novel region of splice-variance.

Fusion may also be made with melanotransferrin (GenBank Acc. NM_{—}013900, murine melanotransferrin). Melanotransferrin is a glycosylated protein found at high levels in malignant melanoma cells and was originally named human melanoma antigen p97 (Brown et al., 1982, Nature, 296: 171-173). It possesses high sequence homology with human serum transferrin, human lactoferrin, and chicken transferrin (Brown et al., 1982, Nature, 296: 171-173; Rose et al., Proc. Natl. Acad. Sci., 1986, 83: 1261-1265). However, unlike these receptors, no cellular receptor has been identified for melanotransferrin. Melanotransferrin reversibly binds iron and it exists in two forms, one of which is bound to cell membranes by a glycosyl phosphatidylinositol anchor while the other form is both soluble and actively secreted (Baker et al., 1992, FEBS Lett, 298: 215-218; Alemany et al., 1993, J. Cell Sci., 104: 1155-1162; Food et al., 1994, J. Biol. Chem. 274: 7011-7017).

Modified Tf fusions may be made with any Tf protein, fragment, domain, or engineered domain. For instance, fusion proteins may be produced using the full-length Tf sequence, with or without the native Tf signal sequence. Trans-bodies may also be made using a single Tf domain, such as an individual N or C domain. In some embodiments, the use of a single N domain is advantageous as the Tf glycosylation sites reside in the C domain and the N domain, on its own, does not bind iron or the Tf receptor. In other embodiments, fusions of a therapeutic protein to a single C domain may be produced, wherein the C domain is altered to reduce, inhibit or prevent glycosylation, iron binding and/or Tf receptor binding.

As used herein, a C terminal domain or lobe modified to function as an N-like domain is modified to exhibit glycosylation patterns or iron binding properties substantially like that of a native or wild-type N domain or lobe. In a preferred embodiment, the C domain or lobe is modified so that it is not glycosylated and does not bind iron by substitution of the relevant C domain regions or amino acids to those present in the corresponding regions or sites of a native or wild-type N domain.

As used herein, a Tf moiety comprising “two N domains or lobes” includes a Tf molecule that is modified to replace the native C domain or lobe with a native or wild-type N domain or lobe or a modified N domain or lobe or contains a C domain that has been modified to function substantially like a wild-type or modified N domain. See U.S. provisional application 60/406,977, which is herein incorporated by reference in its entirety.

Analysis of the two domains by overlay of the two domains (Swiss PDB Viewer 3.7b2, Iterative Magic Fit) and by direct amino acid alignment (ClustalW multiple alignment) reveals that the two domains have diverged over time. Amino acid alignment shows 42% identity and 59% similarity between the two domains. However, approximately 80% of the N domain matches the C domain for structural equivalence. The C domain also has several extra disulfide bonds compared to the N domain.

Alignment of molecular models for the N and C domain reveals the following structural equivalents:

| | | | | | | | | | | | | | | |
|---|---|---|---|---|---|---|---|---|---|---|---|---|---|---|
| N | 4- | 36- | 94- | 138- | 149- | 168- | 178-198 | 219-255 | 259-260 | 263-268 | 271-275 | 279-280 | 283-288 | 309-327 |
| domain | 24 | 72 | 136 | 139 | 164 | 173 | 200-214 | | | | | | 290-304 | |
| (1-330) | | 75- | | | | | | | | | | | | |
| | | 88 | | | | | | | | | | | | |
| C | 340- | 365- | 425- | 470- | 475- | 492- | 507-542 | 555-591 | 593-594 | 597-602 | 605-609 | 614-615 | 620-640 | 645-663 |
| domain | 361 | 415 | 437 | 471 | 490 | 497 | | | | | | | | |
| (340-679) | | | 439- | | | | | | | | | | | |
| | | | 468 | | | | | | | | | | | |

The disulfide bonds for the two domains align as follows:

| N | C |
|---|---|
| | C339-C596 |
| C9-C48 | C345-C377 |
| C19-C39 | C355-C368 |
| | C402-C674 |
| | C418-C637 |
| C118-C194 | C450-C523 |
| C137-C331 | |
| | C474-C665 |
| C158-C174 | C484-C498 |
| C161-C179 | |
| C171-C177 | C495-C506 |
| C227-C241 | C563-C577 |
| | C615-C620 |

Bold aligned disulfide bonds
Italics bridging peptide

In one embodiment, the transferrin portion of the trans-body includes at least two N terminal lobes of transferrin. In further embodiments, the transferrin portion of the trans-body includes at least two N terminal lobes of transferrin derived from human serum transferrin.

In another embodiment, the transferrin portion of the trans-body includes, comprises, or consists of at least two N terminal lobes of transferrin having a mutation in at least one amino acid residue selected from the group consisting of Asp63, Gly65, Tyr95, Tyr188, and His249 of SEQ ID NO: 3.

In another embodiment, the transferrin portion of the modified trans-body includes a recombinant human serum transferrin N-terminal lobe mutant having a mutation at Lys206 or His207 of SEQ ID NO: 3.

In another embodiment, the transferrin portion of the trans-body includes, comprises, or consists of at least two C terminal lobes of transferrin. In further embodiments, the transferrin portion of the trans-body includes at least two C terminal lobes of transferrin derived from human serum transferrin.

In a further embodiment, the C terminal lobe mutant further includes a mutation of at least one of Asn413 and Asn611 of SEQ ID NO: 3 which does not allow glycosylation.

In another embodiment, the transferrin portion of the trans-body includes at least two C terminal lobes of transferrin having a mutation in at least one amino acid residue selected from the group consisting of Asp392, Tyr426, Tyr514, Tyr517 and His585 of SEQ ID NO: 3, wherein the mutant retains the ability to bind metal. In an alternate embodiment, the transferrin portion of the trans-body includes at least two C terminal lobes of transferrin having a mutation in at least one amino acid residue selected from the group consisting of Tyr426, Tyr514, Tyr517 and His585 of SEQ ID NO: 3, wherein the mutant has a reduced ability to bind metal. In another embodiment, the transferrin portion of the trans-body includes at least two C terminal lobes of transferrin having a mutation in at least one amino acid residue selected from the group consisting of Asp392, Tyr426, Tyr517 and His585 of SEQ ID NO:3, wherein the mutant does not retain the ability to bind metal and functions substantially like an N domain.

In some embodiments, the Tf or Tf portion will be of sufficient length to increase the serum stability, in vitro solution stability or bioavailability of the antibody variable region compared to the serum stability (half-life), in vitro stability or bioavailability of antibody variable region in an unfused state. Such an increase in stability, serum half-life or bioavailability may be about a 30%, 50%, 70%, 80%, 90% or more increase over the unfused antibody variable region. In some cases, the trans-bodies comprising modified transferrin exhibit a serum half-life of about 10-20 or more days, about 12-18 days or about 14-17 days.

When the C domain of Tf is part of the trans-body, the two N-linked glycosylation sites, amino acid residues corresponding to N413 and N611 of SEQ ID NO:3 may be mutated for expression in a yeast system to prevent glycosylation or hypermannosylationn and extend the serum half-life of the fusion protein and/or antibody variable region (to produce asialo-, or in some instances, monosialo-Tf or disialo-Tf). In addition to Tf amino acids corresponding to N413 and N611, mutations to the residues within the N-X-S/T glycosylation site to prevent or substantially reduce glycosylation. See U.S. Pat. No. 5,986,067 of Funk et al. It has also been reported that the N domain of Tf expressed in Pichia pastoris becomes O-linked glycosylated with a single hexose at S32 which also may be mutated or modified to prevent such glycosylation.

Accordingly, in one embodiment of the invention, the trans-body includes a modified transferrin molecule wherein the transferrin exhibits reduced glycosylation, including but not limited to asialo- monosialo- and disialo-forms of Tf. In another embodiment, the transferrin portion of the trans-body includes a recombinant transferrin mutant that is mutated to prevent glycosylation. In another embodiment, the transferrin portion of the trans-body includes a recombinant transferrin mutant that is fully glycosylated. In a further embodiment, the transferrin portion of the trans-body includes a recombinant human serum transferrin mutant that is mutated to prevent glycosylation, wherein at least one of Asn413 and Asn611 of SEQ ID NO:3 are mutated to an amino acid which does not allow glycosylation. In another embodiment, the transferrin portion of the trans-body includes a recombinant human serum transferrin mutant that is mutated to prevent or substantially reduce glycosylation, wherein mutations may to the residues within the N-X-S/T glycosylation site.

As discussed below in more detail, modified Tf fusion proteins, preferably trans-bodies comprising a modified Tf, of the invention may also be engineered to not bind iron and/or not bind the Tf receptor. In other embodiments of the invention, the iron binding is retained and the iron binding ability of Tf may be used in two ways, one to deliver a therapeutic protein or peptide(s) to the inside of a cell and/or across the BBB. These embodiments that bind iron and/or the Tf receptor will often be engineered to reduce or prevent glycosylation to extend the serum half-life of the therapeutic protein. The N domain alone will not bind to TfR when loaded with iron, and the iron bound C domain will bind TfR but not with the same affinity as the whole molecule.

In another embodiment, the transferrin portion of the transferrin fusion protein, preferably a trans-body, includes a recombinant transferrin mutant having a mutation wherein the mutant does not retain the ability to bind metal. In an alternate embodiment, the transferrin portion of the transferrin fusion protein includes a recombinant transferrin mutant having a mutation wherein the mutant has a weaker binding avidity for metal than wild-type serum transferrin. In an alternate embodiment, the transferrin portion of the transferrin fusion protein includes a recombinant transferrin mutant having a mutation wherein the mutant has a stronger binding avidity for metal than wild-type serum transferrin.

In another embodiment, the transferrin portion of the trans-body, includes a recombinant transferrin mutant having a mutation wherein the mutant does not retain the ability to bind to the transferrin receptor. In an alternate embodiment, the transferrin portion of the trans-body includes a recombinant transferrin mutant having a mutation wherein the mutant has a weaker binding avidity for the transferrin receptor than wild-type serum transferrin. In an alternate embodiment, the transferrin portion of the trans-body includes a recombinant transferrin mutant having a mutation wherein the mutant has a stronger binding avidity for the transferrin receptor than wild-type serum transferrin.

In another embodiment, the transferrin portion of the trans-body includes a recombinant transferrin mutant having a mutation wherein the mutant does not retain the ability to bind to carbonate. In an alternate embodiment, the transferrin portion of the trans-body includes a recombinant transferrin mutant having a mutation wherein the mutant has a weaker binding avidity for carbonate than wild-type serum transferrin. In an alternate embodiment, the transferrin portion of the trans-body includes a recombinant transferrin mutant having a mutation wherein the mutant has a stronger binding avidity for carbonate than wild-type serum transferrin.

In another embodiment, the transferrin portion of the trans-body includes a recombinant human serum transferrin mutant having a mutation in at least one amino acid residue selected from the group consisting of Asp63, Gly65, Tyr95, Tyr188, His249, Asp392, Tyr426, Tyr514, Tyr517 and His585 of SEQ ID NO: 3, wherein the mutant retains the ability to bind metal. In an alternate embodiment, a recombinant human serum transferrin mutant having a mutation in at least one amino acid residue selected from the group consisting of Asp63, Gly65, Tyr95, Tyr188, His249, Asp392, Tyr426, Tyr514, Tyr517 and His585 of SEQ ID NO: 3, wherein the mutant has a reduced ability to bind metal. In another embodiment, a recombinant human serum transferrin mutant having a mutation in at least one amino acid residue selected from the group consisting of Asp63, Gly65, Tyr95, Tyr188, His249, Asp392, Tyr426, Tyr517 and His585 of SEQ ID NO: 3, wherein the mutant does not retain the ability to bind metal.

In another embodiment, the transferrin portion of the trans-body includes a recombinant human serum transferrin mutant having a mutation at Lys206 or His207 of SEQ ID NO: 3, wherein the mutant has a stronger binding avidity for metal than wild-type human serum transferrin (see U.S. Pat. No. 5,986,067, which is herein incorporated by reference in its entirety). In an alternate embodiment, the transferrin portion of the trans-body includes a recombinant human serum transferrin mutant having a mutation at Lys206 or His207 of SEQ ID NO: 3, wherein the mutant has a weaker binding avidity for metal than wild-type human serum transferrin. In a further embodiment, the transferrin portion of the trans-body includes a recombinant human serum transferrin mutant having a mutation at Lys206 or His207 of SEQ ID NO:3, wherein the mutant does not bind metal.

Any available technique may be used to produce the trans-bodies of the invention, including but not limited to molecular techniques commonly available, for instance, those disclosed in Sambrook et al. Molecular Cloning: A Laboratory Manual, 2nd Ed., Cold Spring Harbor Laboratory Press, 1989. When carrying out nucleotide substitutions using techniques for accomplishing site-specific mutagenesis that are well known in the art, the encoded amino acid changes are preferably of a minor nature, that is, conservative amino acid substitutions, although other, non-conservative, substitutions are contemplated as well, particularly when producing a modified transferrin portion of a trans-body, e.g., a modified trans-body exhibiting reduced glycosylation, reduced iron binding and the like. Specifically contemplated are amino acid substitutions, small deletions or insertions, typically of one to about 30 amino acids; insertions between transferrin domains; small amino- or carboxyl-terminal extensions, such as an amino-terminal methionine residue, or small linker peptides of less than 50, 40, 30, 20 or 10 residues between transferrin domains or linking a transferrin protein and therapeutic protein or peptide, preferably an antibody variable region; or a small extension that facilitates purification, such as a poly-histidine tract, an antigenic epitope or a binding domain.

Examples of conservative amino acid substitutions are substitutions made within the same group such as within the group of basic amino acids (such as arginine, lysine, histidine), acidic amino acids (such as glutamic acid and aspartic acid), polar amino acids (such as glutamine and asparagine), hydrophobic amino acids (such as leucine, isoleucine, valine), aromatic amino acids (such as phenylalanine, tryptophan, tyrosine) and small amino acids (such as glycine, alanine, serine, threonine, methionine).

Non-conservative substitutions encompass substitutions of amino acids in one group by amino acids in another group. For example, a non-conservative substitution would include the substitution of a polar amino acid for a hydrophobic amino acid. For a general description of nucleotide substitution, see e.g. Ford et al. (1991), Prot. Exp. Pur. 2: 95-107. Non-conservative substitutions, deletions and insertions are particularly useful to produce TF fusion proteins, preferably trans-bodies, of the invention that exhibit no or reduced binding of iron, no or reduced binding of the fusion protein to the Tf receptor and/or no or reduced glycosylation.

In the polypeptide and proteins of the invention, the following system is followed for designating amino acids in accordance with the following conventional list:

TABLE OF AMINO ACIDS

| | ONE-LETTER | THREE-LETTER |
|---|---|---|
| AMINO ACID | SYMBOL | SYMBOL |
| Alanine | A | Ala |
| Arginine | R | Arg |
| Asparagine | N | Asn |
| Aspartic Acid | D | Asp |
| Cysteine | C | Cys |
| Glutamine | Q | Gln |
| Glutamic Acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Iron binding and/or receptor binding may be reduced or disrupted by mutation, including deletion, substitution or insertion into, amino acid residues corresponding to one or more of Tf N domain residues Asp63, Tyr95, Tyr188, His249 and/or C domain residues Asp 392, Tyr 426, Tyr 514 and/or His 585. Iron binding may also be affected by mutation to amino acids Lys206, Hys207 or Arg632. Carbonate binding may be reduced or disrupted by mutation, including deletion, substitution or insertion into, amino acid residues corresponding to one or more of Tf N domain residues Thr120, Arg124, Ala126, Gly 127 and/or C domain residues Thr 452, Arg 456, Ala 458 and/or Gly 459. A reduction or disruption of carbonate binding may adversely affect iron and/or receptor binding.

Binding to the Tf receptor may be reduced or disrupted by mutation, including deletion, substitution or insertion into, amino acid residues corresponding to one or more of Tf N domain residues described above for iron binding.

As discussed above, glycosylation may be reduced or prevented by mutation, including deletion, substitution or insertion into, amino acid residues corresponding to one or more of Tf C domain residues within the N-X-S/T sites corresponding to C domain residues N413 and/or N611 (See U.S. Pat. No. 5,986,067). For instance, the N413 and/or N611 may be mutated to Glu residues as may be the adjacent amino acids.

In instances where the Tf fusion proteins, preferably the trans-bodies, of the invention are not modified to prevent glycosylation, iron binding, carbonate binding and/or receptor binding, glycosylation, iron and/or carbonate ions may be stripped from or cleaved off of the fusion protein. For instance, available de-glycosylases may be used to cleave glycosylation residues from the fusion protein, in particular the sugar residues attached to the Tf portion, yeast deficient in glycosylation enzymes may be used to prevent glycosylation and/or recombinant cells may be grown in the presence of an agent that prevents glycosylation, e.g., tunicamycin.

Additional mutations may be made with Tf to alter the three dimensional structure of TF, such as modifications to the hinge region to prevent Tf folding needed for iron biding and Tf receptor recognition. For instance, mutations may be made in or around N domain amino acid residues 94-96, 245-247 and/or 316-318 as well as C domain amino acid residues 425-427, 581-582 and/or 652-658. In addition, mutations may be made in to or around the flanking regions of these sites to alter Tf structure and function.

In one aspect of the invention, the trans-body can function as a carrier protein to extend the half life or bioavailability of the antibody variable region as well as in some instances, delivering the antibody variable region inside a cell and/or across the blood brain barrier. In an alternate embodiment, the trans-body includes a modified transferrin molecule wherein the transferrin does not retain the ability to cross the blood brain barrier.

In another embodiment, the trans-body includes a modified transferrin molecule wherein the transferrin molecule retains the ability to bind to the transferrin receptor and transport the antibody variable region inside cells. In an alternate embodiment, the trans-body includes a modified transferrin molecule wherein the transferrin molecule does not retain the ability to bind to the transferrin receptor and transport the antibody variable region inside cells.

In further embodiments, the trans-body includes a modified transferrin molecule wherein the transferrin molecule retains the ability to bind to the transferrin receptor and transport the antibody variable region inside cells, but does not retain the ability to cross the blood brain barrier. In an alternate embodiment, the trans-body includes a modified transferrin molecule wherein the transferrin molecule retains the ability to cross the blood brain barrier, but does not retain the ability to bind to the transferrin receptor and transport the antibody variable region inside cells.

Modified Transferrin Based Trans-Bodies

The trans-body fusion proteins of the invention may contain one or more copies of the antibody variable region attached to the N-terminus and/or the C-terminus of the Tf protein. In some embodiments, the antibody variable region is attached to both the N- and C-terminus of the Tf protein and the fusion protein may contain one or more equivalents of the antibody variable region on either or both ends of Tf. In other embodiments, the antibody variable region is inserted into known domains of the Tf protein, for instance, into one or more of the loops of Tf (see Ali et al. (1999) J. Biolog. Chem. 274(34):24066-24073). In other embodiments, the antibody variable region is inserted between the N and C domains of Tf.

Generally, the transferrin fusion protein, preferably the trans-body, of the invention may have one modified transferrin-derived region and one antibody variable region. Multiple regions of each protein, however, may be used to make a transferrin fusion protein of the invention. Similarly, more than one antibody variable region may be used to make a transferrin fusion protein of the invention of the invention, thereby producing a multi-functional modified Tf fusion protein.

In one embodiment, the trans-body of the invention contains an antibody variable region or portion thereof fused to a transferrin molecule or portion thereof. In another embodiment, the trans-body of the inventions contains an antibody variable region fused to the N terminus of a transferrin molecule. In an alternate embodiment, the trans-body of the invention contains an antibody variable region fused to the C terminus of a transferrin molecule. In a further embodiment, the trans-body of the invention contains a transferrin molecule fused to the N terminus of an antibody variable region. In an alternate embodiment, the trans-body of the invention contains a transferrin molecule fused to the C terminus of an antibody variable region

In further embodiments, the modified transferrin molecule contains the N terminus of a transferrin molecule fused to what would be the N terminus of an antibody variable region. In an alternate embodiment, the modified transferrin molecule contains the N terminus of a transferrin molecule fused to the C terminus of an antibody variable region. In a further alternate embodiment, the modified transferrin molecule contains the C terminus of a transferrin molecule fused to what would be the C terminus of an antibody variable region. In an alternate embodiment, the modified transferrin molecule contains the C terminus of a transferrin molecule fused to the N terminus of an antibody variable region.

In other embodiments, the trans-body of the inventions contains an antibody variable region fused to both the N-terminus and the C-terminus of modified transferrin. In another embodiment, the antibody variable regions fused at the N- and C-termini bind the same antigens. In an alternate embodiment, the antibody variable regions fused at the N- and C-termini bind different antigens. In another alternate embodiment, the antibody variable regions fused to the N- and C-termini bind different antigens which may be useful for activating two different cells for the treatment or prevention of disease, disorder, or condition. In another embodiment, the antibody variable regions fused at the N- and C-termini bind different antigens which may be useful for bridging two different antigens for the treatment or prevention of diseases or disorders which are known in the art to commonly occur in patients simultaneously.

In addition to modified transferrin fusion protein of the invention, preferably a trans-body comprising a modified transferrin, in which the modified transferrin portion is fused to the N terminal and/or C-terminal of the therapeutic protein portion, preferably the antibody variable region, transferrin fusion protein of the invention may also be produced by inserting the antibody variable region of interest (e.g., a single chain antibody that binds a therapeutic protein or a fragment or variant thereof) into an internal region of the modified transferrin. Internal regions of modified transferrin include, but are not limited to, the loop regions, the iron binding sites, the hinge regions, the bicarbonate binding sites, or the receptor binding domain.

Within the protein sequence of the modified transferrin molecule a number of loops or turns exist, which are stabilized by disulfide bonds. These loops are useful for the insertion, or internal fusion, of therapeutically active peptides, preferably antibody variable regions, particularly those requiring a secondary structure to be functional, or therapeutic proteins, preferably antibody variable region, to essentially generate a modified transferrin molecule with specific biological activity.

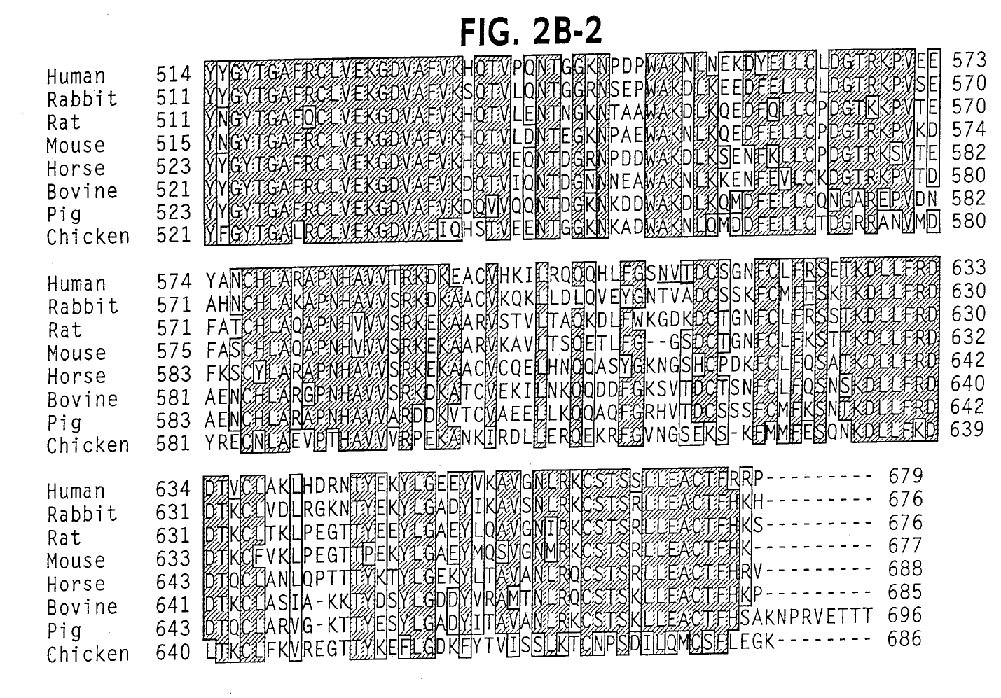

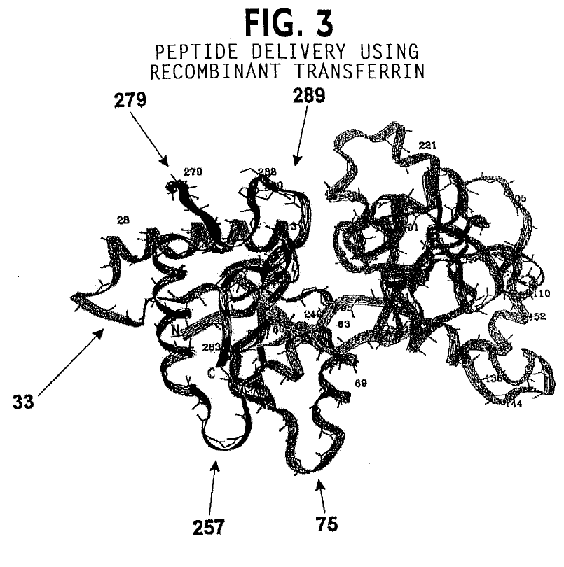

When antibody variable regions, preferably CDRs, are inserted into or replace at least one loop of a Tf molecule, insertions may be made within any of the surface exposed loop regions, in addition to other areas of Tf. For instance, insertions may be made within the loops comprising Tf amino acids 32-33, 74-75, 256-257, 279-280 and 288-289 (Ali et al., supra) (See FIG. 3). As previously described, insertions may also be made within other regions of Tf such as the sites for iron and bicarbonate binding, hinge regions, and the receptor binding domain as described in more detail below. The loops in the Tf protein sequence that are amenable to modification/replacement for the insertion of proteins or peptides may also be used for the development of a screenable library of random peptide inserts. Any procedures may be used to produce nucleic acid inserts for the generation of peptide libraries, including available phage and bacterial display systems, prior to cloning into a Tf domain and/or fusion to the ends of Tf.

The N-terminus of Tf is free and points away from the body of the molecule. Fusions of proteins or peptides on the N-terminus may therefore be a preferred embodiment. Such fusions may include a linker region, such as but not limited to a poly-glycine stretch, to separate the antibody variable region from Tf. Attention to the junction between the leader sequence, the choice of leader sequence, and the structure of the mRNA by codon manipulation/optimization (no major stem loops to inhibit ribosome progress) will increase secretion and can be readily accomplished using standard recombinant protein techniques.

The C-terminus of Tf appears to be more buried and secured by a disulfide bond 6 amino acids from the C-terminus. In human Tf, the C-terminal amino acid is a proline which, depending on the way that it is orientated, will either point a fusion away or into the body of the molecule. A linker or spacer moiety at the C-terminus may be used in some embodiments of the invention.

In one embodiment of the invention, peptides with antigen binding properties can be inserted into transferrin to form trans-bodies. In another embodiment of the invention, any of the trans-bodies can contain an immunogenic peptide that makes the trans-body the target of the immune response. These trans-bodies behave similarly to normal antibodies which can mobilize the immune response after binding to an antigen.

In yet other embodiments, small molecule therapeutics may be complexed with iron and loaded on a modified trans-body for delivery to the inside of cells and across the BBB. The addition of a targeting peptide or, for example, a SCA will target the payload to a particular cell type, e.g., a cancer cell.

Nucleic Acids

The present invention also provides nucleic acid molecules encoding trans-bodies comprising a transferrin protein or a portion of a transferrin protein covalently linked or joined to a therapeutic protein, preferably an antibody variable region. As discussed in more detail above, any antibody variable region may be used. The fusion protein may further comprise a linker region, for instance a linker less than about 50, 40, 30, 20, or 10 amino acid residues. The linker can be covalently linked to and between the transferrin protein or portion thereof and the therapeutic protein, preferably the antibody variable region. Nucleic acid molecules of the invention may be purified or not.

Host cells and vectors for replicating the nucleic acid molecules and for expressing the encoded trans-bodies are also provided. Any vectors or host cells may be used, whether prokaryotic or eukaryotic, but eukaryotic expression systems, in particular yeast expression systems, may be preferred. Many vectors and host cells are known in the art for such purposes. It is well within the skill of the art to select an appropriate set for the desired application.

DNA sequences encoding transferrin, portions of transferrin and antibody variable regions of interest may be cloned from a variety of genomic or cDNA libraries known in the art. The techniques for isolating such DNA sequences using probe-based methods are conventional techniques and are well known to those skilled in the art. Probes for isolating such DNA sequences may be based on published DNA or protein sequences (see, for example, Baldwin, G. S. (1993) Comparison of Transferrin Sequences from Different Species. Comp. Biochem. Physiol. 106B/1:203-218 and all references cited therein, which are hereby incorporated by reference in their entirety). Alternatively, the polymerase chain reaction (PCR) method disclosed by Mullis et al. (U.S. Pat. No. 4,683,195) and Mullis (U.S. Pat. No. 4,683,202), incorporated herein by reference may be used. The choice of library and selection of probes for the isolation of such DNA sequences is within the level of ordinary skill in the art.

As known in the art “similarity” between two polynucleotides or polypeptides is determined by comparing the nucleotide or amino acid sequence and its conserved nucleotide or amino acid substitutes of one polynucleotide or polypeptide to the sequence of a second polynucleotide or polypeptide. Also known in the art is “identity” which means the degree of sequence relatedness between two polypeptide or two polynucleotide sequences as determined by the identity of the match between two strings of such sequences. Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

While there exist a number of methods to measure identity and similarity between two polynucleotide or polypeptide sequences, the terms “identity” and “similarity” are well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIAM J. Applied Math., 48: 1073 (1988). Methods commonly employed to determine identity or similarity between two sequences include, but are not limited to those disclosed in Guide to Huge Computers, Martin J. Bishop, ed., Academic Press, San Diego, 1994, and Carillo, H., and Lipman, D., SIAM J. Applied Math. 48:1073 (1988).

Preferred methods to determine identity are designed to give the largest match between the two sequences tested. Methods to determine identity and similarity are codified in computer programs. Preferred computer program methods to determine identity and similarity between two sequences include, but are not limited to, GCG program package (Devereux, et al., Nucleic Acids Research 12(1):387 (1984)), BLASTP, BLASTN, FASTA (Atschul, et al., J. Molec. Biol. 215:403 (1990)). The degree of similarity or identity referred to above is determined as the degree of identity between the two sequences indicating a derivation of the first sequence from the second. The degree of identity between two nucleic acid sequences may be determined by means of computer programs known in the art such as GAP provided in the GCG program package (Needleman and Wunsch (1970) Journal of Molecular Biology 48:443-453). For purposes of determining the degree of identity between two nucleic acid sequences for the present invention, GAP is used with the following settings: GAP creation penalty of 5.0 and GAP extension penalty of 0.3.

Codon Optimization

The degeneracy of the genetic code permits variations of the nucleotide sequence of a transferrin protein and/or therapeutic protein, preferably an antibody variable region, of interest, while still producing a polypeptide having the identical amino acid sequence as the polypeptide encoded by the native DNA sequence. The procedure, known as “codon optimization” (described in U.S. Pat. No. 5,547,871 which is incorporated herein by reference in its entirety) provides one with a means of designing such an altered DNA sequence. The design of codon optimized genes should take into account a variety of factors, including the frequency of codon usage in an organism, nearest neighbor frequencies, RNA stability, the potential for secondary structure formation, the route of synthesis and the intended future DNA manipulations of that gene. In particular, available methods may be used to alter the codons encoding a given fusion protein with those most readily recognized by yeast when yeast expression systems are used.

The degeneracy of the genetic code permits the same amino acid sequence to be encoded and translated in many different ways. For example, leucine, serine and arginine are each encoded by six different codons, while valine, proline, threonine, alanine and glycine are each encoded by four different codons. However, the frequency of use of such synonymous codons varies from genome to genome among eukaryotes and prokaryotes. For example, synonymous codon-choice patterns among mammals are very similar, while evolutionarily distant organisms such as yeast (S. cerevisiae), bacteria (such as E. coli) and insects (such as D. melanogaster) reveal a clearly different pattern of genomic codon use frequencies (Grantham, R., et al., Nucl. Acids Res., 8, 49-62 (1980); Grantham, R., et al., Nucl. Acids Res., 9, 43-74 (1981); Maroyama, T., et al., Nucl. Acids Res., 14, 151-197 (1986); Aota, S., et al., Nucl. Acids Res., 16, 315-402 (1988); Wada, K., et al., Nucl. Acids Res., 19 Supp., 1981-1985 (1991); Kurland, C. G., FEBS Letters, 285, 165-169 (1991)). These differences in codon-choice patterns appear to contribute to the overall expression levels of individual genes by modulating peptide elongation rates. (Kurland, C. G., FEBS Letters, 285, 165-169 (1991); Pedersen, S., EMBO J., 3, 2895-2898 (1984); Sorensen, M. A., J. Mol. Biol., 207, 365-377 (1989); Randall, L. L., et al., Eur. J. Biochem., 107, 375-379 (1980); Curran, J. F., and Yarus, M., J. Mol. Biol., 209, 65-77 (1989); Varenne, S., et al., J. Mol, Biol., 180, 549-576 (1984), Varenne, S., et al., J. Mol, Biol., 180, 549-576 (1984); Garel, J.-P., J. Theor. Biol., 43, 211-225 (1974); Ikemura, T., J. Mol. Biol., 146, 1-21 (1981); Ikemura, T., J. Mol. Biol., 151, 389-409 (1981)).

The preferred codon usage frequencies for a synthetic gene should reflect the codon usages of nuclear genes derived from the exact (or as closely related as possible) genome of the cell/organism that is intended to be used for recombinant protein expression, particularly that of yeast species. As discussed above, in one preferred embodiment the human Tf sequence is codon optimized, before or after modification as herein described for yeast expression as may be the nucleotide sequence of the antibody variable region.

Vectors

Expression units for use in the present invention will generally comprise the following elements, operably linked in a 5′ to 3′ orientation: a transcriptional promoter, a secretory signal sequence, a DNA sequence encoding a modified Tf fusion protein comprising transferrin protein or a portion of a transferrin protein joined to a DNA sequence encoding a therapeutic protein or peptide of interest, preferably an antibody variable region, and a transcriptional terminator. As discussed above, any arrangement of the therapeutic protein or peptide fused to or within the Tf portion may be used in the vectors of the invention. The selection of suitable promoters, signal sequences and terminators will be determined by the selected host cell and will be evident to one skilled in the art and are discussed more specifically below.

Suitable yeast vectors for use in the present invention are described in U.S. Pat. No. 6,291,212 and include YRp7 (Struhl et al., Proc. Natl. Acad. Sci. USA 76: 1035-1039, 1978), YEp13 (Broach et al., Gene 8: 121-133, 1979), pJDB249 and pJDB219 (Beggs, Nature 275:104-108, 1978), pPPC0005, pSeCHSA, pScNHSA, pC4 and derivatives thereof. Useful yeast plasmid vectors also include pRS403-406, pRS413-416 and the Pichia vectors available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, 7RPI, LEU2 and URA3. Plasmids pRS413˜41.6 are Yeast Centromere plasmids (Ycps).

Such vectors will generally include a selectable marker, which may be one of any number of genes that exhibit a dominant phenotype for which a phenotypic assay exists to enable transformants to be selected. Preferred selectable markers are those that complement host cell auxotrophy, provide antibiotic resistance or enable a cell to utilize specific carbon sources, and include LEU2 (Broach et al. ibid.), URA3 (Botstein et al., Gene 8: 17, 1979), HIS3(Struhl et al., ibid.) or POT1 (Kawasaki and Bell, EP 171,142). Other suitable selectable markers include the CAT gene, which confers chloramphenicol resistance on yeast cells. Preferred promoters for use in yeast include promoters from yeast glycolytic genes (Hitzeman et al., J Biol. Chem. 225: 12073-12080, 1980; Alber and Kawasaki, J. Mol. Appl. Genet. 1: 419-434, 1982; Kawasaki, U.S. Pat. No. 4,599,311) or alcohol dehydrogenase genes (Young et al., in Genetic Engineering of Microorganisms for Chemicals, Hollaender et al., (eds.), p. 355, Plenum, N.Y., 1982; Ammerer, Meth. Enzymol. 101: 192-201, 1983). In this regard, particularly preferred promoters are the TPI1 promoter (Kawasaki, U.S. Pat. No. 4,599,311) and the ADH2-4^{C} (see U.S. Pat. No. 6,291,212) promoter (Russell et al., Nature 304: 652-654, 1983). The expression units may also include a transcriptional terminator. A preferred transcriptional terminator is the TPI1 terminator (Alber and Kawasaki, ibid.).

In addition to yeast, modified fusion proteins of the present invention can be expressed in filamentous fungi, for example, strains of the fungi Aspergillus. Examples of useful promoters include those derived from Aspergillus nidulans glycolytic genes, such as the ADH3 promoter (McKnight et al., EMBO J. 4: 2093-2099, 1985) and the tpiA promoter. An example of a suitable terminator is the ADH3 terminator (McKnight et al., ibid.). The expression units utilizing such components may be cloned into vectors that are capable of insertion into the chromosomal DNA of Aspergillus, for example.

Mammalian expression vectors for use in carrying out the present invention will include a promoter capable of directing the transcription of the modified Tf fusion protein, preferably a trans-body comprising a modified Tf. Preferred promoters include viral promoters and cellular promoters. Preferred viral promoters include the major late promoter from adenovirus 2 (Kaufman and Sharp, Mol. Cell. Biol. 2: 1304-13199, 1982) and the SV40 promoter (Subramani et al., Mol. Cell. Biol. 1: 854-864, 1981). Preferred cellular promoters include the mouse metallothionein-1 promoter (Palmiter et al., Science 222: 809-814, 1983) and a mouse Vκ (see U.S. Pat. No. 6,291,212) promoter (Grant et al., Nuc. Acids Res. 15: 5496, 1987). A particularly preferred promoter is a mouse V_{H} (see U.S. Pat. No. 6,291,212) promoter. Such expression vectors may also contain a set of RNA splice sites located downstream from the promoter and upstream from the DNA sequence encoding the transferrin fusion protein. Preferred RNA splice sites may be obtained from adenovirus and/or immunoglobulin genes.

Also contained in the expression vectors is a polyadenylation signal located downstream of the coding sequence of interest. Polyadenylation signals include the early or late polyadenylation signals from SV40 (Kaufman and Sharp, ibid.), the polyadenylation signal from the adenovirus 5 E1B region and the human growth hormone gene terminator (DeNoto et al., Nuc. Acids Res. 9: 3719-3730, 1981). A particularly preferred polyadenylation signal is the V_{H} (see U.S. Pat. No. 6,291,212) gene terminator. The expression vectors may include a noncoding viral leader sequence, such as the adenovirus 2 tripartite leader, located between the promoter and the RNA splice sites. Preferred vectors may also include enhancer sequences, such as the SV40 enhancer and the mouse μ (see U.S. Pat. No. 6,291,212) enhancer (Gillies, Cell 33: 717-728, 1983). Expression vectors may also include sequences encoding the adenovirus VA RNAs.

Transformation

Techniques for transforming fungi are well known in the literature, and have been described, for instance, by Beggs (ibid.), Hinnen et al. (Proc. Natl. Acad. Sci. USA 75: 1929-1933, 1978), Yelton et al., (Proc. Natl. Acad. Sci. USA 81: 1740-1747, 1984), and Russell (Nature 301: 167-169, 1983). The genotype of the host cell will generally contain a genetic defect that is complemented by the selectable marker present on the expression vector. Choice of a particular host and selectable marker is well within the level of ordinary skill in the art.

Cloned DNA sequences comprising modified Tf fusion proteins of the invention may be introduced into cultured mammalian cells by, for example, calcium phosphate-mediated transfection (Wigler et al., Cell 14: 725, 1978; Corsaro and Pearson, Somatic Cell Genetics 7: 603, 1981; Graham and Van der Eb, Virology 52: 456, 1973.) Other techniques for introducing cloned DNA sequences into mammalian cells, such as electroporation (Neumann et al., EMBO J. 1: 841-845, 1982), or lipofection may also be used. In order to identify cells that have integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. A preferred amplifiable selectable marker is the DHFR gene. A particularly preferred amplifiable marker is the DHFR^{r} (see U.S. Pat. No. 6,291,212) cDNA (Simonsen and Levinson, Proc. Natl. Acad. Sci. USA 80: 2495-2499, 1983). Selectable markers are reviewed by Thilly (Mammalian Cell Technology, Butterworth Publishers, Stoneham, Mass.) and the choice of selectable markers is well within the level of ordinary skill in the art.

Host Cells

The present invention also includes a cell, preferably a yeast cell transformed to express a modified transferrin fusion protein of the invention. In addition to the transformed host cells themselves, the present invention also includes a culture of those cells, preferably a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away.

Host cells for use in practicing the present invention include eukaryotic cells, and in some cases prokaryotic cells, capable of being transformed or transfected with exogenous DNA and grown in culture, such as cultured mammalian, insect, fungal, plant and bacterial cells.

Fungal cells, including species of yeast (e.g., Saccharomyces spp., Schizosaccharomyces spp., Pichia spp.) may be used as host cells within the present invention. Exemplary genera of yeast contemplated to be useful in the practice, of the present invention as hosts for expressing the transferrin fusion protein, preferably the trans-body, of the inventions are Pichia (formerly classified as Hansenula), Saccharomyces, Kluyveromyces, Aspergillus, Candida, Torulopsis, Torulaspora, Schizosaccharomyces, Citeromyces, Pachysolen, Zygosaecharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopyis, and the like. Examples of Saccharomyces spp. are S. cerevisiae, S. italicus and S. rouxii. Examples of Kluyveromyces spp. are K. ftagilis, K. lactis and K. marxianus. A suitable Tórulasppra species is T. delbrueckii. Examples of Pichia (Hansenula) spp. are P. angusta (formerly H. polymorpha), P. anomala (formerly H. anomala) and P. pastoris.

Particularly useful host cells to produce the Tf fusion proteins, preferably trans-bodies, of the invention are the methanoltrophic Pichia pastoris (Steinlein et al. (1995) Protein Express. Purif. 6:619-624). Pichia pastoris has been developed to be an outstanding host for the production of foreign proteins since its alcohol oxidase promoter was isolated and cloned; its transformation was first reported in 1985. P. pastoris can utilize methanol as a carbon source in the absence of glucose. The P. pastoris expression system can use the methanol-induced alcohol oxidase (AOX1) promoter, which controls the gene that codes for the expression of alcohol oxidase, the enzyme which catalyzes the first step in the metabolism of methanol. This promoter has been characterized and incorporated into a series of P. pastoris expression vectors. Since the proteins produced in P. pastoris are typically folded correctly and secreted into the medium, the fermentation of genetically engineered P. pastoris provides an excellent alternative to E. coli expression systems. A number of proteins have been produced using this system, including tetanus toxin fragment, Bordatella pertussis pertactin, human serum albumin, lysozyme, interferon alpha, and glycosylated and non-glycosylated transferrin.

The transformation of F. oxysporum may, for instance, be carried out as described by Malardier et al. (1989) Gene 78:147-156.

Strains of the yeast Saccharomyces cerevisiae are another preferred host. In a preferred embodiment, a yeast cell, or more specifically, a Saccharomyces cerevisiae host cell that contains a genetic deficiency in a gene required for asparagine-linked glycosylation of glycoproteins is used. S. cerevisiae host cells having such defects may be prepared using standard techniques of mutation and selection, although many available yeast strains have been modified to prevent or reduce glycosylation or hypermannosylation. Ballou et al. (J. Biol. Chem. 255: 5986-5991, 1980) have described the isolation of mannoprotein biosynthesis mutants that are defective in genes which affect asparagine-linked glycosylation.

To optimize production of the heterologous proteins, it is also preferred that the host strain carries a mutation, such as the S. cerevisiae pep4 mutation (Jones, Genetics 85: 23-33, 1977), which results in reduced proteolytic activity. Host strains containing mutations in other protease encoding regions are particularly useful to produce large quantities of the Tf fusion proteins of the invention.

Host cells containing DNA constructs of the present invention are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals and growth factors. The growth medium will generally select for cells containing the DNA construct by, for example, drug selection or deficiency in an essential nutrient which are complemented by the selectable marker on the DNA construct or co-transfected with the DNA construct. Yeast cells, for example, are preferably grown in a chemically defined medium, comprising a non-amino acid nitrogen source, inorganic salts, vitamins and essential amino acid supplements. The pH of the medium is preferably maintained at a pH greater than 2 and less than 8, preferably at pH 6.5. Methods for maintaining a stable pH include buffering and constant pH control, preferably through the addition of sodium hydroxide. Preferred buffering agents include succinic acid and Bis-Tris (Sigma Chemical Co., St. Louis, Mo.). Yeast cells having a defect in a gene required for asparagine-linked glycosylation are preferably grown in a medium containing an osmotic stabilizer. A preferred osmotic stabilizer is sorbitol supplemented into the medium at a concentration between 0.1 M and 1.5 M., preferably at 0.5 M or 1.0 M.

Cultured mammalian cells are generally grown in commercially available serum-containing or serum-free media. Selection of a medium appropriate for the particular cell line used is within the level of ordinary skill in the art. Transfected mammalian cells are allowed to grow for a period of time, typically 1-2 days, to begin expressing the DNA sequence(s) of interest. Drug selection is then applied to select for growth of cells that are expressing the selectable marker in a stable fashion. For cells that have been transfected with an amplifiable selectable marker the drug concentration may be increased in a stepwise manner to select for increased copy number of the cloned sequences, thereby increasing expression levels.

Baculovirus/insect cell expression systems may also be used to produce the modified Tf fusion proteins of the invention. The BacPAK™ Baculovirus Expression System (BD Biosciences (Clontech) expresses recombinant proteins at high levels in insect host cells. The target gene is inserted into a transfer vector, which is cotransfected into insect host cells with the linearized BacPAK6 viral DNA. The BacPAK6 DNA is missing an essential portion of the baculovirus genome. When the DNA recombines with the vector, the essential element is restored and the target gene is transferred to the baculovirus genome. Following recombination, a few viral plaques are picked and purified, and the recombinant phenotype is verified. The newly isolated recombinant virus can then be amplified and used to infect insect cell cultures to produce large amounts of the desired protein.

Secretory Signal Sequences

The terms “secretory signal sequence” or “signal sequence” or “secretion leader sequence” are used interchangeably and are described, for example in U.S. Pat. Nos. 6,291,212 and 5,547,871, both of which are herein incorporated by reference in their entirety. Secretory signal sequences or signal sequences or secretion leader sequences encode secretory peptides. A secretory peptide is an amino acid sequence that acts to direct the secretion of a mature polypeptide or protein from a cell. Secretory peptides are generally characterized by a core of hydrophobic amino acids and are typically (but not exclusively) found at the amino termini of newly synthesized proteins. Very often the secretory peptide is cleaved from the mature protein during secretion. Secretory peptides may contain processing sites that allow cleavage of the signal peptide from the mature protein as it passes through the secretory pathway. Processing sites may be encoded within the signal peptide or may be added to the signal peptide by, for example, in vitro mutagenesis.

Secretory peptides may be used to direct the secretion of modified Tf fusion proteins of the invention. One such secretory peptide that may be used in combination with other secretory peptides is the third domain of the yeast Barrier protein. Secretory signal sequences or signal sequences or secretion leader sequences are required for a complex series of post-translational processing steps which result in secretion of a protein. If an intact signal sequence is present, the protein being expressed enters the lumen of the rough endoplasmic reticulum and is then transported through the Golgi apparatus to secretory vesicles and is finally transported out of the cell. Generally, the signal sequence immediately follows the initiation codon and encodes a signal peptide at the amino-terminal end of the protein to be secreted. In most cases, the signal sequence is cleaved off by a specific protease, called a signal peptidase. Preferred signal sequences improve the processing and export efficiency of recombinant protein expression using viral, mammalian or yeast expression vectors. In some cases, the native Tf signal sequence may be used to express and secrete fusion proteins of the invention.

Linkers

The Tf moiety and the antibody variable region of the modified transferrin fusion proteins of the invention can be fused directly or using a linker peptide of various lengths to provide greater physical separation and allow more spatial mobility between the fused proteins and thus maximize the accessibility of the antibody variable region, for instance, for binding to its cognate receptor. The linker peptide may consist of amino acids that are flexible or more rigid. For example, a linker such as but not limited to a poly-glycine stretch. The linker can be less than about 50, 40, 30, 20, or 10 amino acid residues. The linker can be covalently linked to and between the transferrin protein or portion thereof and the antibody variable region.

Linkers are also used to join the antibody variable regions. Suitable linkers for joining the antibody variable regions are those that allow the antibody variable regions to fold into a three dimensional structure that maintains the binding specificity of a whole antibody.

Detection of Trans-Bodies

Assays for detection of biologically active modified transferrin-trans-body may include Western transfer, protein blot or colony filter as well as activity based assays that detect the fusion protein comprising transferrin and antibody variable region. A Western transfer filter may be prepared using the method described by Towbin et al. (Proc. Natl. Acad. Sci. USA 76: 4350-4354, 1979). Briefly, samples are electrophoresed in a sodium dodecylsulfate polyacrylamide gel. The proteins in the gel are electrophoretically transferred to nitrocellulose paper. Protein blot filters may be prepared by filtering supernatant samples or concentrates through nitrocellulose filters using, for example, a Minifold (Schleicher & Schuell, Keene, N.H.). Colony filters may be prepared by growing colonies on a nitrocellulose filter that has been laid across an appropriate growth medium. In this method, a solid medium is preferred. The cells are allowed to grow on the filters for at least 12 hours. The cells are removed from the filters by washing with an appropriate buffer that does not remove the proteins bound to the filters. A preferred buffer comprises 25 mM Tris-base, 19 mM glycine, pH 8.3, 20% methanol.

Transferrin fusion proteins, preferably trans-bodies, of the present invention may be labeled with a radioisotope or other imaging agent and used for in vivo diagnostic purposes. Preferred radioisotope imaging agents include iodine-125 and technetium-99, with technetium-99 being particularly preferred. Methods for producing protein-isotope conjugates are well known in the art, and are described by, for example, Eckelman et al. (U.S. Pat. No. 4,652,440), Parker et al. (WO 87/05030) and Wilber et al. (EP 203,764). Alternatively, the trans-bodies may be bound to spin label enhancers and used for magnetic resonance (MR) imaging. Suitable spin label enhancers include stable, sterically hindered, free radical compounds such as nitroxides. Methods for labeling ligands for MR imaging are disclosed by, for example, Coffman et al. (U.S. Pat. No. 4,656,026). For administration, the labeled trans-bodies are combined with a pharmaceutically acceptable carrier or diluent, such as sterile saline or sterile water. Administration is preferably by bolus injection, preferably intravenously.

Detection of a trans-body of the present invention can be facilitated by coupling (i.e., physically linking) to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In one embodiment where one is assaying for the ability of a trans-body of the invention to bind or compete with an antibody for binding to an antigen, various immunoassays known in the art can be used, including but not limited to, competitive and non-competitive assay systems using techniques such as radioimmunoassays, ELISA (enzyme linked immunosorbent assay), sandwich immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays), complement fixation assays, immunofluorescence assays, protein A assays, and immunoelectrophoresis assays, etc. In one embodiment, the binding of the trans-body is detected by detecting a label on the trans-body. In another embodiment, the trans-body is detected by detecting binding of a secondary antibody or reagent that interacts with the trans-body. In a further embodiment, the secondary antibody is labeled. Many means are known in the art for detecting binding in an immunoassay and are within the scope of the present invention.

Production of Trans-Bodies

The present invention further provides methods for producing a modified fusion protein, preferably trans-body comprising a modified Tf using nucleic acid molecules herein described. In general terms, the production of a recombinant form of a protein typically involves the following steps.

A nucleic acid molecule is first obtained that encodes a trans-body of the invention. The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be accomplished in a variety of ways. For example, the construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier and are otherwise known to persons skilled in the art. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce a desired recombinant protein.

As discussed above, any expression system may be used, including yeast, bacterial, animal, plant, eukaryotic and prokaryotic systems. In some embodiments, yeast, mammalian cell culture and transgenic animal or plant production systems are preferred. In other embodiments, yeast systems that have been modified to reduce native yeast glycosylation, hyper-glycosylation or proteolytic activity may be used.

Isolation/Purification of Trans-Bodies

Secreted, biologically active, modified transferrin fusion proteins, preferably trans-bodies comprising a modified transferrin, may be isolated from the medium of host cells grown under conditions that allow the secretion of the biologically active fusion proteins. The cell material is removed from the culture medium, and the biologically active fusion proteins are isolated using isolation techniques known in the art. Suitable isolation techniques include precipitation and fractionation by a variety of chromatographic methods, including gel filtration, ion exchange chromatography and affinity chromatography.

A particularly preferred purification method is affinity chromatography on an iron binding or metal chelating column or an immunoaffinity chromatography using the cognate antigen directed against the antibody variable region of the polypeptide fusion. The antigen is preferably immobilized or attached to a solid support or substrate. A particularly preferred substrate is CNBr-activated Sepharose (Pharmacia LKB Technologies, Inc., Piscataway, N.J.). By this method, the medium is combined with the antigen/substrate under conditions that will allow binding to occur. The complex may be washed to remove unbound material, and the trans-body is released or eluted through the use of conditions unfavorable to complex formation. Particularly useful methods of elution include changes in pH, wherein the immobilized antigen has a high affinity for the trans-body at a first pH and a reduced affinity at a second (higher or lower) pH; changes in concentration of certain chaotropic agents; or through the use of detergents.

Delivery of a Trans-Body to the Inside of a Cell and/or Across the Blood Brain Barrier (BBB)

Within the scope of the invention, the modified trans-bodies may be used as a carrier to deliver a molecule or small molecule therapeutic complexed to the ferric ion of transferrin to the inside of a cell or across the blood brain barrier. In these embodiments, the transferrin will typically be engineered or modified to inhibit, prevent or remove glycosylation to extend the serum half-life of the trans-body and/or antibody variable region. The addition of a targeting peptide or, for example, a single chain antibody is specifically contemplated to further target the trans-body to a particular cell type, e.g., a cancer cell.

In one embodiment, the iron-containing, anti-anemic drug, ferric-sorbitol-citrate complex is loaded onto a modified Tf fusion protein of the invention. Ferric-sorbitol-citrate (FSC) has been shown to inhibit proliferation of various murine cancer cells in vitro and cause tumor regression in vivo, while not having any effect on proliferation of non-malignant cells (Poljak-Blazi et al. (June 2000) Cancer Biotherapy and Radiopharmaceuticals (United States), 15/3:285-293).

In another embodiment, the antineoplastic drug adriamycin (Doxorubicin) and/or the chemotherapeutic drug bleomycin, both of which are known to form complexes with ferric ion, is loaded onto a trans-body of the invention. In other embodiments, a salt of a drug, for instance, a citrate or carbonate salt, may be prepared and complexed with the ferric iron that is then bound to Tf. As tumor cells often display a higher turnover rate for iron; transferrin modified to carry at least one anti-tumor agent, may provide a means of increasing agent exposure or load to the tumor cells. (Demant, E. J., (1983) Eur. J. of Biochem. 137/(1-2):113-118; Padbury et al. (1985) J. Biol. Chem. 260/13:7820-7823).

Pharmaceutical Formulations and Treatment Methods

The modified fusion proteins, preferably trans-bodies comprising a modified transferrin, of the invention may be administered to a patient in need thereof using standard administration protocols. For instance, the modified Tf fusion proteins of the present invention can be provided alone, or in combination, or in sequential combination with other agents that modulate a particular pathological process. As used herein, two agents are said to be administered in combination when the two agents are administered simultaneously or are administered independently in a fashion such that the agents will act at the same or near the same time.

The agents of the present invention can be administered via parenteral, subcutaneous, intravenous, intramuscular, intraperitoneal, transdermal and buccal routes. For example, an agent may be administered locally to a site of injury via microinfusion. Alternatively, or concurrently, administration may be noninvasive by either the oral, inhalation, nasal, or pulmonary route. The dosage administered will be dependent upon the age, health, and weight of the recipient, kind of concurrent treatment, if any, frequency of treatment, and the nature of the effect desired.

The present invention further provides compositions containing one or more trans-bodies of the invention. While individual needs vary, determination of optimal ranges of effective amounts of each component is within the skill of the art. Typical dosages comprise about 1 pg/kg to about 100 mg/kg body weight. The preferred dosages for systemic administration comprise about 100 ng/kg to about 100 mg/kg body weight or about 100-200 mg of protein/dose. The preferred dosages for direct administration to a site via microinfusion comprise about 1 ng/kg to about 1 mg/kg body weight. When administered via direct injection or microinfusion, modified fusion proteins of the invention may be engineered to exhibit reduced or no binding of iron to prevent, in part, localized iron toxicity.

In addition to the pharmacologically active trans-body, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient. Suitable formulations for oral administration include hard or soft gelatin capsules, pills, tablets, including coated tablets, elixirs, suspensions, syrups or inhalations and controlled release forms thereof.

In practicing the methods of this invention, the trans-bodies of this invention may be used alone or in combination, or in combination with other therapeutic or diagnostic agents. In certain preferred embodiments, the trans-bodies of this invention may be co-administered along with other compounds typically prescribed for these conditions according to generally accepted medical practice. The trans-bodies of this invention can be utilized in vivo, ordinarily in mammals, such as humans, sheep, horses, cattle, pigs, dogs, cats, rats and mice, or in vitro.

Transgenic Animals

The production of transgenic non-human animals that contain a modified transferrin fusion construct, preferably a trans-body, with increased serum half-life increased serum stability or increased bioavailability of the instant invention is contemplated in one embodiment of the present invention. In some embodiments, lactoferrin may be used as the Tf portion of the fusion protein so that the fusion protein is produced and secreted in milk.

The successful production of transgenic, non-human animals has been described in a number of patents and publications, such as, for example U.S. Pat. No. 6,291,740 (issued Sep. 18, 2001); U.S. Pat. No. 6,281,408 (issued Aug. 28, 2001); and U.S. Pat. No. 6,271,436 (issued Aug. 7, 2001) the contents of which are hereby incorporated by reference in their entireties.

The ability to alter the genetic make-up of animals, such as domesticated mammals including cows, pigs, goats, horses, cattle, and sheep, allows a number of commercial applications. These applications include the production of animals which express large quantities of exogenous proteins in an easily harvested form (e.g., expression into the milk or blood), the production of animals with increased weight gain, feed efficiency, carcass composition, milk production or content, disease resistance and resistance to infection by specific microorganisms and the production of animals having enhanced growth rates or reproductive performance. Animals which contain exogenous DNA sequences in their genome are referred to as transgenic animals.

The most widely used method for the production of transgenic animals is the microinjection of DNA into the pronuclei of fertilized embryos (Wall et al., J. Cell. Biochem. 49:113 [1992]). Other methods for the production of transgenic animals include the infection of embryos with retroviruses or with retroviral vectors. Infection of both pre- and post-implantation mouse embryos with either wild-type or recombinant retroviruses has been reported (Janenich, Proc. Natl. Acad. Sci. USA 73:1260 [1976]; Janenich et al., Cell 24:519 [1981]; Stuhlmann et al., Proc. Natl. Acad. Sci. USA 81:7151 [1984]; Jahner et al., Proc. Natl. Acad Sci. USA 82:6927 [1985]; Van der Putten et al., Proc. Natl. Acad Sci. USA 82:6148-6152 [1985]; Stewart et al., EMBO J. 6:383-388 [1987]).

An alternative means for infecting embryos with retroviruses is the injection of virus or virus-producing cells into the blastocoele of mouse embryos (Jahner, D. et al., Nature 298:623 [1982]). The introduction of transgenes into the germline of mice has been reported using intrauterine retroviral infection of the midgestation mouse embryo (Jahner et al., supra [1982]). Infection of bovine and ovine embryos with retroviruses or retroviral vectors to create transgenic animals has been reported. These protocols involve the micro-injection of retroviral particles or growth arrested (i.e., mitomycin C-treated) cells which shed retroviral particles into the perivitelline space of fertilized eggs or early embryos (PCT International Application WO 90/08832 [1990]; and Haskell and Bowen, Mol. Reprod. Dev., 40:386 [1995]. PCT International Application WO 90/08832 describes the injection of wild-type feline leukemia virus B into the perivitelline space of sheep embryos at the 2 to 8 cell stage. Fetuses derived from injected embryos were shown to contain multiple sites of integration.

U.S. Pat. No. 6,291,740 (issued Sep. 18, 2001) describes the production of transgenic animals by the introduction of exogenous DNA into pre-maturation oocytes and mature, unfertilized oocytes (i.e., pre-fertilization oocytes) using retroviral vectors which transduce dividing cells (e.g., vectors derived from murine leukemia virus [MLV]). This patent also describes methods and compositions for cytomegalovirus promoter-driven, as well as mouse mammary tumor LTR expression of various recombinant proteins.

U.S. Pat. No. 6,281,408 (issued Aug. 28, 2001) describes methods for producing transgenic animals using embryonic stem cells. Briefly, the embryonic stem cells are used in a mixed cell co-culture with a morula to generate transgenic animals. Foreign genetic material is introduced into the embryonic stem cells prior to co-culturing by, for example, electroporation, microinjection or retroviral delivery. ES cells transfected in this manner are selected for integrations of the gene via a selection marker such as neomycin.

U.S. Pat. No. 6,271,436 (issued Aug. 7, 2001) describes the production of transgenic animals using methods including isolation of primordial germ cells, culturing these cells to produce primordial germ cell-derived cell lines, transforming both the primordial germ cells and the cultured cell lines, and using these transformed cells and cell lines to generate transgenic animals. The efficiency at which transgenic animals are generated is greatly increased, thereby allowing the use of homologous recombination in producing transgenic non-rodent animal species.

Gene Therapy

The use of modified transferrin fusion constructs for gene therapy wherein a modified transferrin protein or transferrin domain is joined to a antibody variable domain is contemplated in one embodiment of this invention. The modified transferrin fusion constructs with increased serum half-life or serum stability of the instant invention are ideally suited to gene therapy treatments.

The successful use of gene therapy to express a soluble fusion protein has been described. Briefly, gene therapy via injection of an adenovirus vector containing a gene encoding a soluble fusion protein consisting of cytotoxic lymphocyte antigen 4 (CTLA4) and the Fc portion of human immunoglubulin G1 was recently shown in Ijima et al. (Jun. 10, 2001) Human Gene Therapy (United States) 12/9:1063-77. In this application of gene therapy, a murine model of type II collagen-induced arthritis was successfully treated via intraarticular injection of the vector.

Gene therapy is also described in a number of U.S. patents including U.S. Pat. No. 6,225,290 (issued May 1, 2001); U.S. Pat. No. 6,187,305 (issued Feb. 13, 2001); and U.S. Pat. No. 6,140,111 (issued Oct. 31, 2000).

U.S. Pat. No. 6,225,290 provides methods and constructs whereby intestinal epithelial cells of a mammalian subject are genetically altered to operatively incorporate a gene which expresses a protein which has a desired therapeutic effect. Intestinal cell transformation is accomplished by administration of a formulation composed primarily of naked DNA, and the DNA may be administered orally. Oral or other intragastrointestinal routes of administration provide a simple method of administration, while the use of naked nucleic acid avoids the complications associated with use of viral vectors to accomplish gene therapy. The expressed protein is secreted directly into the gastrointestinal tract and/or blood stream to obtain therapeutic blood levels of the protein thereby treating the patient in need of the protein. The transformed intestinal epithelial cells provide short or long term therapeutic cures for diseases associated with a deficiency in a particular protein or which are amenable to treatment by overexpression of a protein.

U.S. Pat. No. 6,187,305 provides methods of gene or DNA targeting in cells of vertebrate, particularly mammalian, origin. Briefly, DNA is introduced into primary or secondary cells of vertebrate origin through homologous recombination or targeting of the DNA, which is introduced into genomic DNA of the primary or secondary cells at a preselected site.

U.S. Pat. No. 6,140,111 (issued Oct. 31, 2000) describes retroviral gene therapy vectors. The disclosed retroviral vectors include an insertion site for genes of interest and are capable of expressing high levels of the protein derived from the genes of interest in a wide variety of transfected cell types. Also disclosed are retroviral vectors lacking a selectable marker, thus rendering them suitable for human gene therapy in the treatment of a variety of disease states without the co-expression of a marker product, such as an antibiotic. These retroviral vectors are especially suited for use in certain packaging cell lines. The ability of retroviral vectors to insert into the genome of mammalian cells have made them particularly promising candidates for use in the genetic therapy of genetic diseases in humans and animals. Genetic therapy typically involves (1) adding new genetic material to patient cells in vivo, or (2) removing patient cells from the body, adding new genetic material to the cells and reintroducing them into the body, i.e., in vitro gene therapy. Discussions of how to perform gene therapy in a variety of cells using retroviral vectors can be found, for example, in U.S. Pat. No. 4,868,116, issued Sep. 19, 1989, and U.S. Pat. No. 4,980,286, issued Dec. 25, 1990 (epithelial cells), WO89/07136 published Aug. 10, 1989 (hepatocyte cells), EP 378,576 published Jul. 25, 1990 (fibroblast cells), and WO89/05345 published Jun. 15, 1989 and WO/90/06997, published Jun. 28, 1990 (endothelial cells), the disclosures of which are incorporated herein by reference.

Trans-Bodies Comprising Antibody Variable Regions Against Toxins

The present invention provides trans-bodies comprising transferrin or modified transferrin and antibody variable regions against toxins. As used herein the term “toxin” refers to a poisonous substance of biological origin. The trans-bodies comprising one or more antibody variable region of a desired toxin antibody and a transferrin may be obtained as discussed above. Trans-bodies comprising antibody variable regions against toxins may be used to treat patients suffering from diseases associated with toxins. The trans-bodies comprising an antibody variable region against a toxin and a transferrin or modified transferrin molecule also may be used for diagnostic purposes.

Toxins are produced by various microorganisms. Examples of such microorganisms include, but are not limited to: Corynebacterium diphtheriae, Staphylococci, Salmonella typhimuium, Shigellae, Pseudomonas aeruginosa, Vibrio cholerae, Clostridium botulinum, Clostridium tetani, Clostridium difficile, Clostridium perfringens, Clostridium welchii, Yersiniae pestis, Eschericia coli, and Bacillus anthracis. Examples of toxins produced by these microorganisms include, but are not limited to, Pseudomonas exotoxins (PE), Diphtheria toxins (DT), ricin toxin, abrin toxin, anthrax toxins, shiga toxin, botulism toxin, tetanus toxin, cholera toxin, maitotoxin, palytoxin, ciguatoxin, textilotoxin, batrachotoxin, alpha conotoxin, taipoxin, tetrodotoxin, alpha tityustoxin, saxitoxin, anatoxin, microcystin, aconitine, exfoliatin toxins A and B, enterotoxins, toxic shock syndrome toxin (TSST-1), Y. pestis toxin, gas gangrene toxin, and others.

Toxins can be separated into various groups such as, but not limited to, ADP-ribosylating toxins, exfoliatin toxins, staphylococcal enterotoxins, and metalloproteases. Examples of ADP-ribosylating toxins include Pseudomonas toxin A, diptheria toxin, pertussis toxin, and cholera toxin.

The exfoliatin toxins A and B, the staphylococcal enterotoxins, and the toxic shock syndrome toxin, TSST-1, belong to the growing family of microbial superantigens that activate T cells and monocytes/macrophages, resulting in the production of cytokines that mediate local or systemic effects depending on the amount of toxin formed, the immune status of the host, and the access of the toxin to the circulation. The exfoliatin toxins mediate the dermatologic manifestations of the staphylococcal scalded-skin syndrome and bullous impetigo. These toxins cause intraepidermal cleavage of the skin at the stratum granulosum, leading to bullae formation and denudation. Seven distinct enterotoxins (A, B, C1, C2, C3, D, and E) have been implicated in food poisoning due to S. aureus. These toxins enhance intestinal peristalsis and appear to induce vomiting by a direct effect on the central nervous system. Toxic shock syndrome (TSS) is most commonly mediated by TSST-1, which is present in 5 to 25 percent of clinical isolates of S. aureus. TSS is also mediated less frequently by enterotoxin B and, rarely, enterotoxin C1.

Examples of metalloproteases include biological toxins derived from Clostridial species (C. botulinum and C. tetani) and Bacillus anthracis (Herreros et al. In The Comprehensive Sourcebook of Bacterial Protein Toxins. J. E. Alouf and J. H. Freer, Eds. 2^{nd} edition, San Diego, Academic Press, 1999; pp 202-228.). These bacteria express and secrete zinc metalloproteases that enter eukaryotic cells and specifically cleave distinct target proteins.

The genus Clostridium is comprised of gram-positive, anaerobic, spore-forming bacilli. The natural habitat of these organisms is the environment and the intestinal tracts of humans and other animals. Indeed, clostridia are ubiquitous; they are commonly found in soil, dust, sewage, marine sediments, decaying vegetation, and mud. (See e.g., Sneath et al., “Clostridium,” Bergey's Manual.RTM. of Systematic Bacteriology, Vol. 2, pp. 1141-1200, Williams & Wilkins [1986]). Despite the identification of approximately 100 species of Clostridium, only a small number have been recognized as etiologic agents of medical and veterinary importance. Nonetheless, these species are associated with very serious diseases, including botulism, tetanus, anaerobic cellulitis, gas gangrene, bacteremia, pseudomembranous colitis, and clostridial gastroenteritis. Table 1 lists some of the species of medical and veterinary importance and the diseases with which they are associated.

As indicated in Table 1, the majority of these organisms may be associated with serious and/or debilitating disease. In most cases, the pathogenicity of these organisms is related to the release of powerful exotoxins or highly destructive enzymes. Indeed, several species of the genus Clostridium produce toxins and other enzymes of great medical and veterinary significance (C. L. Hatheway, Clin. Microbiol. Rev. 3:66-98 (1990)).

TABLE 2
Clostridium Species Of Medical And Veterinary Importance*

| Species | Disease |
|---|---|
| C. aminovalericum | Bacteriuria (pregnant women) |
| C. argentinese | Infected wounds; Bacteremia; Botulism; |
| | Infections of amniotic fluid |
| C. baratii | Infected war wounds; Peritonitis; |
| | Infectious processes of the eye, ear and prostate |
| C. beijerinckikii | Infected wounds |
| C. bifermentans | Infected wounds; Abscesses; Gas Gangrene; Bacteremia |
| C. botulinum | Food poisoning; Botulism (wound, food, infant) |
| C. butyricum | Urinary tract, lower respiratory tract, pleural cavity, and |
| | abdominal infections; Infected wounds; Abscesses; |
| | Bacteremia |
| C. cadaveris | Abscesses; Infected wounds |
| C. carnis | Soft tissue infections; Bacteremia |
| C. chauvoei | Blackleg |
| C. clostridioforme | Abdominal, cervical, scrotal, pleural, and other |
| | Infections; Septicemia; Peritonitis; Appendicitis |
| C. cochlearium | Isolated from human disease processes, but role in |
| | disease unknown. |
| C. difficile | Antimicrobial-associated diarrhea; Pseudomembranous |
| | enterocolitis; Bacteremia; Pyogenic infections |
| C. fallax | Soft tissue infections |
| C. ghnoii | Soft tissue infections |
| C. glycolicum | Wound infections; Abscesses; Peritonitis |
| C. hastiforme | Infected war wounds; Bacteremia; Abscesses |
| C. histolyticum | Infected war wounds; Gas gangrene; |
| | Gingival plaque isolate |
| C. indolis | Gastrointestinal tract infections |
| C. innocuum | Gastrointestinal tract infections; Empyema |
| C. irregulare | Penile lesions |
| C. leptum | Isolated from human disease processes, but role in |
| | disease unknown. |
| C. limosum | Bacteremia; Peritonitis; Pulmonary infections |
| C. malenominatum | Various infectious processes |
| C. novyi | Infected wounds; Gas gangrene; Blackleg, Big head |
| | (ovine); Redwater disease (bovine) |
| C. oroticum | Urinary tract infections; Rectal abscesses. |
| C. paraputrificum | Bacteremia; Peritonitis; Infected wounds; Appendicitis |
| C. perfringens | Gas gangrene; Anaerobic cellulitis; Intra-abdominal |
| | abscesses; Soft tissue infections; Food poisoning; |
| | Necrotizing pneumonia; Empyema; Meningitis; |
| | Bacteremia; Uterine Infections; Enteritis necrotans; |
| | Lamb dysentery; Struck; Ovine Enterotoxemia |
| C. putrefaciens | Bacteriuria (Pregnant women with bacteremia) |
| C. putrificum | Abscesses; Infected wounds; Bacteremia |
| C. ramosum | Infections of the abdominal cavity, genital tract, lung, |
| | and biliary tract; Bacteremia |
| C. sartagoforme | Isolated from human disease processes, but role in |
| | disease unknown. |
| C. septicum | Gas gangrene; Bacteremia; Suppurative infections; |
| | Necrotizing enterocolitis; Braxy |
| C. sordellii | Gas gangrene; Wound infections; Penile lesions; |
| | Bacteremia; Abscesses; Abdominal and vaginal |
| | infections |
| C. sphenoides | Appendicitis; Bacteremia; Bone and soft tissue |
| | infections; Intraperitoneal infections; Infected war |
| | wounds; Visceral gas gangrene; Renal abscesses |
| C. sporogenes | Gas gangrene; Bacteremia; Endocarditis; central nervous |
| | system and pleuropulmonary infections; Penile lesions; |
| | Infected war wounds; Other pyogenic infections |
| C. subterminale | Bacteremia; Empyema; Biliary tract, soft tissue and |
| | bone infections |
| C. symbiosum | Liver abscesses; Bacteremia; Infections resulting due to |
| | bowel flora |
| C. tertium | Gas gangrene; Appendicitis; Brain abscesses; Intestinal |
| | tract and soft tissue infections; Infected war wounds; |
| | Periodontitis; Bacteremia |
| C. tetani | Tetanus; Infected gums and teeth; Corneal ulcerations; |
| | Mastoid and middle ear infections; Intraperitoneal |
| | infections; Tetanus neonatorum; Postpartum uterine |
| | infections; Soft tissue infections, especially related to |
| | trauma (including abrasions and lacerations); Infections |
| | related to use of contaminated needles |
| C. thermosaccharolyticum | Isolated from human disease processes, but role in |
| | disease unknown. |
| *Compiled from Engelkirk et al. “Classification”, Principles and Practice of Clinical Anaerobic Bacteriology, pp. 22-23, Star Publishing Co., Belmont, CA (1992); Stephen and Petrowski, “Toxins Which Traverse Membranes and Deregulate Cells,” in Bacterial Toxins, 2d ed., pp. 66-67, American Society for Microbiology (1986); Berkow and Fletcher (eds.), “Bacterial Diseases,” Merck Manual of Diagnosis and | |
| # Therapy, 16th ed., pp. 116-126, Merck Research Laboratories, Rahway, N.J. (1992); and Siegmond and Fraser (eds.), “Clostridial Infections,” Merck Veterinary Manual, 5th ed., pp. 396-409, Merck & Co., Rahway, N.J. (1979). | |

Because of their significance for human and veterinary medicine, much research has been conducted on these toxins, in particular those of C. botulinum, C. tetani, and C. peringens, and C. difficile.

The clostridial neurotoxins are potent inhibitors of calcium-dependent neurotransmitter secretion in neuronal cells. They are currently considered to mediate this activity through a specific endoproteolytic cleavage of at least one of three vesicle or pre-synaptic membrane associated proteins VAMP, syntaxin or SNAP-25 which are central to the vesicle docking and membrane fusion events of neurotransmitter secretion. The neuronal cell targeting of tetanus and botulinum neurotoxins is considered to be a receptor mediated event following which the toxins become internalized and subsequently traffic to the appropriate intracellular compartment where they effect their endopeptidase activity.

Clostridium Botulinum Toxin

The anaerobic, gram positive bacterium Clostridium botulinum produces the most poisonous biological neurotoxin known with a lethal human dose in the nanogram range. The effect of the toxin ranges from diarrheal diseases that can cause destruction of the colon, to paralytic effects that can cause death. The spores of Clostridium botulinum are found in soil and can grow in improperly sterilized and sealed food containers of home based canneries, which are the cause of many of the cases of botulism. The symptoms of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulism disease may be grouped into four types, based on the method of introduction of toxin into the bloodstream. Food-borne botulism results from ingesting improperly preserved and inadequately heated food that contains botulinal toxin (i.e., the toxin is pre-formed prior to ingestion). Wound-induced botulism results from C. botulinum penetrating traumatized tissue and producing toxin that is absorbed into the bloodstream. Since 1950, thirty cases of wound botulism have been reported (Swartz, “Anaerobic Spore-Forming Bacilli: The Clostridia,” pp. 633-646, in Davis et al., (eds.), Microbiology, 4th edition, J. B. Lippincott Co. (1990)). Inhalation botulism results when the toxin is inhaled. Inhalation botulism has been reported as the result of accidental exposure in the laboratory (Holzer, Med. Klin., 41:1735 [1962]) and is a potential danger if the toxin is used as an agent of biological warfare (Franz et al., in Botulinum and Tetanus Neurotoxins, DasGupta (ed.), Plenum Press, New York [1993], pp. 473-476). Infectious infant botulism results from C. botulinum colonization of the infant intestine with production of toxin and its absorption into the bloodstream.

Different strains of Clostridium botulinum each produce antigenically distinct toxin designated by the letters A-G. Serotype A toxin has been implicated in 26% of the cases of food botulism; types B, E, and F have also been implicated in a smaller percentage of the food botulism cases (Sugiyama, Microbiol. Rev., 44:419 (1980)). Wound botulism has been reportedly caused by only types A or B toxins (Sugiyama, supra). Nearly all cases of infant botulism have been caused by bacteria producing either type A or type B toxin (exceptionally, one New Mexico case was caused by Clostridium botulinum producing type F toxin and another by Clostridium botulinum producing a type B-type F hybrid) (Arnon, Epidemiol. Rev., 3:45 (1981)). Type C toxin affects waterfowl, cattle, horses and mink. Type D toxin affects cattle, and type E toxin affects both humans and birds.

Various antitoxins against C. botulinum toxin have been used. A trivalent antitoxin derived from horse plasma is commercially available from Connaught Industries Ltd. as a therapy for toxin types A, B, and E. A heptavalent equine botulinal antitoxin which uses only the F(ab′)2 portion of the antibody molecule has been tested by the United States Military (Balady, USAMRDC Newsletter, p. 6 (1991)). This was raised against impure toxoids in those large animals and is not a high titer preparation. A pentavalent human antitoxin has been collected from immunized human subjects for use as a treatment for infant botulism. Immunization of subjects with toxin preparations has been done in an attempt to induce immunity against botulinal toxins. A C. botulinum vaccine comprising chemically inactivated (i.e., formaldehyde-treated) type A, B, C, D and E toxin is commercially available for human usage. However, these antitoxins are neither safe nor effective for the treatment of botulism disease.

Clostridium Tetani Toxin

Although tetanus has been recognized since ancient times (e.g., the disease was described by Hippocrates), it was not hypothesized to have an infectious agent as its cause until 1867 (See e.g., Hatheway, supra, at p. 75). The strictly toxigenic disease caused by C. tetani is often associated with puncture wounds that do not appear to be serious. The organism is readily isolated from a variety of sources, including soil and the intestinal contents of many animal species (e.g., humans, horses, etc.).

Disease results upon the production of toxin by the organism at a site of trauma. The toxin rapidly binds to neural tissue, resulting in the paralysis and spasms characteristic of tetanus. Largely due to the availability of effective toxoids, tetanus is now largely a disease of non-immunized animals, including humans. For example, neonatal tetanus due to contamination of the umbilical stump is very prevalent in some areas of the world. Neonatal tetanus is almost always severe and is highly fatal. Approximately one half of the cases reported worldwide are neonatal tetanus.

Tetanus is an extremely dramatic disease resulting from the action of the potent neurotoxin (tetanospasmin). The toxin binds to gangliosides in the central nervous system, and blocks inhibitory impulses to the motor neurons, resulting in prolonged muscle spasms of both flexor and extensor muscles. C. tetani also produces “tetanolysin,” an oxygen-sensitive hemolysis that is functionally and serologically related to streptolysin O, and the oxygen-sensitive hemolysis of various other organisms, including at least six Clostridium species (See e.g., Hatheway, at p. 76). This toxin lyses a variety of cells, including erythrocytes, polymorphonuclear leukocytes, macrophages, fibroblasts, ascites tumor cells, HeLa cells, and platelets. It has an affinity for cholesterol and related sterols. Although in experimental studies, the toxin has been shown to cause pulmonary edema and death in mice, intravascular hemolysis in rabbits and monkeys, and cardiotoxic effects in monkeys, its role in C. tetani infections remains in question (See, Hatheway, at p. 77).

Although the diagnosis of tetanus is relatively easy in advanced cases, successful treatment depends upon early diagnosis before a lethal amount of toxin can become fixed to neural tissue. Thus, patients are usually treated empirically, prior to receiving laboratory data. Tetanus toxoid is used prophylactically to prevent disease. For immunosuppressed patients who may not respond to prophylactic injections of toxoid, human tetanus immunoglobulin given intramuscularly may be used.

Treatment of diagnosed tetanus involves debridement of the wound to remove the organism from the wound site. This debridement occurs after the patient's spasms have been controlled by benzodiazepines. Penicillin or metronidazole is often used to treat the patient. Human tetanus immunoglobulin is also administered intramuscularly. Supportive treatment (e.g., respiratory assistance, nutritional support and intravenous fluids) is often crucial in patient survival. In cases of clean, minor wounds, tetanus toxoid is administered if the patient has not had a booster dose within the past 10 years, although for serious wounds, toxoid is administered if the patient has not had a booster within the past five years.

Clostridium Perfringens Toxin

C. perfringens is reported to be the most widely occurring pathogenic bacterium (See, Hatheway, supra, at p. 77). The organism, first described by Welch and Nuttall in 1892, and named Bacillus aerogenes capsulatus, has also been commonly referred to as C. welchii. C. perfringens is commonly isolated from soil samples, as well as the intestinal contents of humans and other animals. Although other Clostridium species are also associated with gas gangrene (e.g., C. novyi, C. septicum, C. histolyticum, C. tertium, C. bifermentans, and C. sporogenes), C. perfringens is the species most commonly involved. These organisms are not highly pathogenic when introduced into healthy tissue, but are associated with rapidly progressive, devastating infections characterized by the accumulation of gas and extensive muscle and tissue necrosis, when introduced in the presence of tissue injury (e.g., damaged muscle). During active multiplication, invasive strains of clostridia produce exotoxins with necrotizing (i.e., cytolytic), hemolytic, and/or lethal properties. In addition, enzymes such as collagenase proteinase, deoxyribonuclease, and hyaluronidase produced by the organisms result in the accumulation of toxic degradation products in the tissues.

C. perfringens produces four major lethal toxins (alpha, beta, epsilon, and iota), upon which the toxin types of the species are based, as well as nine minor toxins (or soluble antigens), that may or may not be involved in the pathogenicity associated with the organism (See, Hatheway, supra, at 77). These minor toxins are delta, theta, kappa, lambda, mu, nu, gamma, eta, and neuraminidase. In addition, some strains produce an enterotoxin that is responsible for C. perfringens food-borne disease. C. perfringens may be divided into “toxin types” designated as A, B, C, D, and E, based on the toxins produced. For example, most strains of toxin type A produce the alpha toxin, but not the other major lethal toxins (i.e., beta, epsilon, and iota); toxin type B organisms produce all of the major lethal toxins with the exception of iota toxin; toxin type C organisms produce alpha and beta major lethal toxins, but not epsilon or iota toxins; toxin type D organisms produce alpha and epsilon toxins, but not beta or iota toxins; and toxin type E organisms produce alpha and iota toxins, but not beta or epsilon toxins.

The alpha toxin is a lecithinase (phospholipase C), while the beta toxin is a necrotizing, trypsin-labile toxin; the epsilon toxin is a permease, trypsin-activatable toxin; and iota toxin is a dermonecrotic, binary, ADP-ribosylating, trypsin-activatable toxin. The delta toxin is a hemolysin; the theta toxin is an oxygen-labile hemolysin, and cytolysin; the kappa toxin is a collagenase and gelatinase; the lambda toxin is a protease; the mu toxin is a hyaluronidase; and the nu toxin is a DNase. The gamma and eta toxins have not been well-characterized and their existence is questionable (See, Hatheway, supra, at p. 77). The neuraminidase is an N-acetylneuraminic acid glycohydrolase, and the enterotoxin is enterotoxic and cytotoxic.

The various toxins are commonly associated with particular diseases. For example, toxin type A organisms are associated with myonecrosis (gas gangrene), food-borne illness, and infectious diarrhea in humans, enterotoxemia of lambs, cattle, goats, horses, dogs, alpacas, and other animals; necrotic enteritis in fowl; equine intestinal clostridiosis; acute gastric dilation in non-human primates, and various other animal species, including humans. Toxin type B organisms are associated with lamb dysentery, ovine and caprine enterotoxemia (particularly in Europe and the Middle East), and guinea pig enterotoxemia. Toxin type C organisms are associated with Darmbrand (Germany), and pig-bel (New Guinea), struck in sheep, lamb and pig enterotoxemia, and necrotic enteritis in fowl. Toxin type D organisms are associated with enterotoxemia of sheep, and pulpy kidney disease in lambs. Toxin type E organisms are associated with calf enterotoxemia, lamb dysentery, guinea pig enterotoxemia, and rabbit “iota” enterotoxemia. While C. perfringens type A strains are commonly isolated from soil samples, and is also readily found in intestinal contents in the absence of disease, type B, C, D, and E strains apparently do not survive in soils (i.e., these strains are obligate parasites).

Currently, treatment of contaminated wounds involves prompt surgical debridement of contaminated wounds to prevent anaerobic cellulitis. Gas gangrene, as antimicrobial therapy alone is insufficient. Once a clostridial wound infection has become established, prompt surgical debridement is necessary. In cases of anaerobic cellulitis, wide excision of the affected area and debridement are required, while gas gangrene usually requires complete extirpation of the involved muscle (i.e., usually amputation of the limb is necessary).

High doses of penicillin are usually administered, although the emergence of penicillin-resistant strains has resulted in the use of clindamycin, chloramphenicol, and metronidazole. However, strains resistant to tetracycline, chloramphenicol, erythromycin, and clindamycin have been observed. Polyvalent equine antitoxin prepared against toxic filtrates of four species (C. perfringens, C novyi, C. septicum, and C. histolyticum) has been used in the prophylaxis and treatment of gas gangrene. However, its efficacy was not established and it is no longer available for clinical use (Swartz, p 645, in Davis et al. (eds.), Microbiology, 4th edition, J. B. Lippincott Co. (1990)).

Clostridium Difficile Toxin

Clostridium difficile, an organism which gained its name due to difficulties encountered in its isolation, has recently been proven to be an etiologic agent of diarrheal disease. (Sneath et al., p. 1165.). Clostridium difficile is the etiological agent of pseudomembranous colitis in humans and animals. C. difficile is associated with a range of diarrhetic illness, ranging from diarrhea alone to marked diarrhea and necrosis of the gastrointestinal mucosa with the accumulation of inflammatory cells and fibrin, which forms a pseudomembrane in the affected area.

The enterotoxicity of C. difficile is primarily due to the action of two toxins, designated A and B, each of approximately 300,000 in molecular weight. Both are potent cytotoxins, with toxin A possessing direct enterocytotoxic activity (Lyerly et al., Infect. Immun. 60:4633 (1992)). Unlike toxin A of C. perfringens, an organism rarely associated with antimicrobial-associated diarrhea, the toxin of C. difficile is not a spore coat constituent and is not produced during sporulation (Swartz, at p. 644.). C. difficile toxin A causes hemorrhage, fluid accumulation and mucosal damage in rabbit ileal loops and appears to increase the uptake of toxin B by the intestinal mucosa. Toxin B does not cause intestinal fluid accumulation, but it is 1000 times more toxic than toxin A to tissue culture cells and causes membrane damage. Although both toxins induce similar cellular effects such as actin disaggregation, differences in cell specificity occurs.

Both toxins are important in disease (Borriello et al., Rev. Infect. Dis., 12(suppl. 2):S185 (1990); Lyerly et al., Infect. Immun., 47:349 (1985); and Rolfe, Infect. Immun., 59:1223 (1990)). Toxin A is thought to act first by binding to brush border receptors, destroying the outer mucosal layer, then allowing toxin B to gain access to the underlying tissue. These steps in pathogenesis would indicate that the production of neutralizing antibodies against toxin A may be sufficient in the prophylactic therapy of CDAD. However, antibodies against toxin B may be a necessary additional component for an effective therapeutic against later stage colonic disease. Indeed, it has been reported that animals require antibodies to both toxin A and toxin B to be completely protected against the disease (Kim and Rolfe, Abstr. Ann. Meet. Am. Soc. Microbiol., 69:62 (1987)).

U.S. Pat. No. 5,071,759 discloses a monoclonal antibody that immunologically binds both toxin A and toxin B of Clostridium difficile. U.S. Pat. No. 6,365,158 discloses methods for generating neutralizing antitoxin directed against Clostridium difficile toxin B. In particular, the antitoxin directed against these toxins is produced in avian species using soluble recombinant Clostridium difficile toxin B. This avian antitoxin is designed so as to be orally administrable in therapeutic amounts and may be in any form (i.e., as a solid or in aqueous solution).

Bacillus Anthracis Toxin

Anthrax toxin is a well-known agent of biological warfare derived from Bacillus anthracis. Bacillus anthracis produces three proteins which when combined appropriately form two potent toxins, collectively designated anthrax toxin. Protective antigen (PA, 82,684 Da (Dalton)) and edema factor (EF, 89,840 Da) combine to form edema toxin (ET), while PA and lethal factor (LF, 90,237 Da) combine to form lethal toxin (LT) (Leppla, S. H. Alouf, J. E. and Freer, J. H., eds. Academic Press, London 277-302, 1991). ET and LT each conform to the AB toxin model, with PA providing the target cell binding (B) function and EF or LF acting as the effector or catalytic (A) moieties. A unique feature of these toxins is that LF and EF have no toxicity in the absence of PA, apparently because they cannot gain access to the cytosol of eukaryotic cells.

PA is capable of binding to the surface of many types of cells. After PA binds to a specific receptor (Leppla, supra, 1991) on the surface of susceptible cells, it is cleaved at a single site by a cell surface protease, probably furin, to produce an amino-terminal 19-kDa fragment that is released from the receptor/PA complex (Singh et al., J. Biol. Chem. 264:19103-19107, 1989). Removal of this fragment from PA exposes a high-affinity binding site for LF and EF on the receptor-bound 63-kDa carboxyl-terminal fragment (PA63). The complex of PA63 and LF or EF enters cells and probably passes through acidified endosomes to reach the cytosol.

Recently, two of the targets of Lethal factor (LF) were identified in cells. LF is a metalloprotease that specifically cleaves Mek1 and Mek2 proteins, kinases that are part of the MAP-kinase signaling pathway. LF's proteolytic activity inactivates the MAP-kinase signaling cascade through cleavage of mitogen activated protein kinase kinases 1 or 2 (MEK1 or MEK2). (Leppla, S. A. In The Comprehensive Sourcebook of Bacterial Protein Toxins. J. E. Alouf and J. H. Freer, Eds. 2^{nd} edition, San Diego, Academic Press, 1999; pp243-263.).

PA, the non-toxic, cell-binding component of the toxin, is the essential component of the currently available human vaccine. The vaccine is usually produced from batch cultures of the Sterne strain of B. anthracis, which although avirulent, is still required to be handled as a Class III pathogen. In addition to PA, the vaccine contains small amounts of the anthrax toxin moieties, edema factor and lethal factor, and a range of culture derived proteins. All these factors contribute to the recorded reactogenicity of the vaccine in some individuals. The vaccine is expensive and requires a six month course of four vaccinations. Futhermore, present evidence suggests that this vaccine may not be effective against inhalation challenge with certain strains (M. G. Broster et al., Proceedings of the International Workshop on Anthrax, Apr. 11-13, 1989, Winchester UK. Salisbury med Bull Suppl No 68, (1990) 91-92).

U.S. Pat. No. 6,267,966 provides a recombinant microorganism which is able to express Bacillus anthracis protective antigen or a variant or fragment thereof which is able to generate an immune response in a mammal, said microorganism comprising a sequence which encodes PA or said variant or fragment thereof wherein either (i) a gene of said microorganism which encodes a catabolic repressor protein and/or AbrB is inactivated, and/or (ii) a region of the said PA sequence which can act as a catabolic repressor binding site is inactivated; and/or (iii) a region of the said PA sequence which can act as an AbrB binding site is inactivated.

Antibodies Against Toxins

U.S. Pat. No. 6,440,408 provides a vaccine preparation comprising a live organism (bacteria or protozoa) complexed with neutralizing antibodies specific to that organism. The amount of complexed neutralizing antibodies is such that the organism remains capable of inducing an active immune response, while at the same time providing some degree of protection against the deleterious effects of the pathogen.

Bacterial or protozoal neutralizing antibodies are those which combat the infectivity of a bacteria or protozoa in vivo if the bacteria or protozoa and the antibodies are allowed to react together for a sufficient time. The source of the bacterial or protozoal neutralizing antibody is not critical. They may originate from any animal, including birds (e.g., chicken, turkey) and mammals (e.g., rat, rabbit, goat, horse). The bacterial or protozoal neutralizing antibodies may be polyclonal or monoclonal in origin. See, e.g., D. Yelton and M. Scharff, 68 American Scientist 510 (1980). The antibodies may be chimeric. See, e.g., M. Walker et al., 26 Molecular Immunology 403 (1989).

Bacteria that may be used in generating antibodies include, but are not limited to, Actinobacillosis lignieresi, Actinomyces bovis, Aerobacter aerogenes, Anaplasma marginale, Bacillus anthracis, Borrelia anserina, Brucella canis, Clostridium chauvoei, C. hemolyticium C. novyi, C perfringens, C. septicum, C. tetani, Corynebacterium equi, C. pyogenes, C. renale, Cowdria ruminantium, Dermatophilus congolensis, Erysipelothrix insidiosa, Escherichia coli, Fusiformis necrophorus, Haemobartonella canis, Hemophilus spp. H. suis, Leptospira spp., Moraxella bovis, Mycoplasma spp. M. hyopneumoniae, Nanophyetus salmincola, Pasteurella anatipestifer, P. hemolytica, P. multocida, Salmonella abortus-ovis, Shigella equirulis, Staphylococcus aureus, S. hyicus. S. hyos, Streptococcus agalactiae, S. dysgalactiae, S. equi, S. uberis, and Vibrio fetus (for the corresponding diseases, see Veterinary Pharmacology and Therapeutics 5th Edition, pg 746 Table 50.2 (N. Booth and L. McDonald Eds., 1982) (Iowa State University Press); and Corynebacterium diptheriae, Mycobacterium bovis, M. leprae, M. tuberculosis, Nocardia asteroides, Bacillus anthracis, Clostridium botulinum, C. difficile, C. perfringens, C. tetani, Staphylococcus aureus, Streptococcus pneumoniae, S. pyogenes, Bordetella pertusiss, Psudomonas aeruginos, Campylobacter jejuni, Brucella spp., Francisella tularenssis, Legionella pneumophila, Chlamydia psittaci. C. trachomatis, Escherichia coli, Klebsiella pneumoniae, Salmonella typhi, S. typhimurium, Yersinia enterocolitica, Y. pestis, Vibrio cholerae, Haemophilus influenza, Mycoplasma pneumoniae, Neiseseria gonorrhoeae, N. meninigitidis, Coxiella burneti, Rickettsia mooseria, R. prowazekii, R. rickettsii, R. tsutsugamushi, Borrelia spp., Leptospira interrogans, Treponema pallidum, and Listeria monocytogenes (for the corresponding diseases see R. Stanier et al., The Microbial World, pg. 637-38 Table 32.3 (5th Edition 1986).

U.S. Pat. No. 4,689,299 teaches the production of stable hybrid cell lines that secrete human monoclonal antibodies against bacterial toxins by fusing post-immunization human peripheral blood lymphocytes with nonsecretor mouse myeloma cell. The patent discloses method of generating protective monoclonal antibodies against tetanus toxin and diphtheria toxin that bind tetanus toxin and diphtheria toxin in vitro, respectively, and prevent tetanus and diphtheria in vivo in animals, respectively. The anti-tetanus toxin and anti-diphtheria toxin human monoclonal antibodies of the present invention can neutralize tetanus toxin and diphtheria toxin, respectively. They can prevent tetanus and diphtheria disease, and hence represent new chemotherapeutic agents for the prevention and/or treatment of toxin-induced diseases.

Trans-Bodies Comprising Antigenic Immune-Modulating Regions

In one embodiment of the invention, the trans-bodies are further modified to include at least one antigenic or immune modulating peptide. One or more of these peptides can be incorporated into the transferrin or modified transferrin. Trans-bodies containing one or more antigenic regions not only can bind their antigens, but can also induce an immune response in the host. The cellular and humoral responses induced by these trans-bodies are stronger than standard antibodies because most hosts are already immunized with and have memory to the antigenic determinant incorporated in the trans-bodies.

The antigenic peptide has a chain length of minimally six amino acids, preferably 12 amino acids (considering the three amino acids on either side thereof) and can contain an infinitely long chain of amino acids or their components, which can be characterized by the presence of other epitopes of the same or different antigen or allergen. Where it is free of such additional chain with or without such additional epitopes, it generally does not have an amino acid chain exceeding 50 amino acids. Where a short chain is desired containing the desired epitope, it preferably does not have an amino acid chain length greater than 40, more especially not greater than 30 and more particularly not greater than 20 amino acids. Most preferably, the trans-body has a peptide of 15-30 amino acids.

Preferably, the trans-bodies are incorporated with antigenic regions that induce an immune response. More preferably, the antigenic regions are peptides that are known to be highly antigenic, including the antigenic regions are selected from proteins that have been used for vaccines. In other embodiments, the peptides inserted on or into a trans-body are capable of modulating the immune system. For instance, antibody Fc regions may be included in the trans-bodies of the invention.

The immunogenicity of a polypeptide can be defined as the immune response directed against a limited number of immunogenic determinants, which are confined to a few loci on the polypeptide molecule, (see Crumpton, M. J., in The Antigens (ed. Sela, M., Academic Press, New York, 1974); Benjamini, E. et al., Curr. Topics Microbiol. Immunol. 58 85-135 (1972); Atassi, M. Z., Immunochemistry 12, 423-438 (1975)). Antisera prepared against chemically synthesized peptides corresponding to short linear stretches of the polypeptide sequence have been shown to react well with the whole polypeptide, (see Green, N. et al., Cell 28, 477-487 (1982); Bittle, J. L. et al., Nature 298, 30-33 (1982); Dreesman et al., Nature 295, 158-160 (1982); Prince, A. M., Ikram, H., Hopp, T. P., Proc. Nat. Acad. Sci. USA 79, 579-582 (1982); Lerner, R. A. et al., Proc. Nat. Acad. Sci. USA 78, 3403-3407 (1981); Neurath, A. R., Kent, S. B. H., Strick, N., Proc. Nat. Acad. Sci. USA 79, 7871-7875 (1982)). However, interactions have been found to occur even when the site of interaction does not correlate with the immunogenic determinants of the native protein, (see Green, N., et al, Supra). Conversely, since antibodies produced against the native protein are by definition directed to the immunogenic determinants, it follows that a peptide interacting with these antibodies must contain at least a part of an immunogenic determinant.

From a study of the few proteins for which the immunogenic determinants have been accurately mapped, it is clear that a determinant can consist of a single sequence, (continuous), or of several sequences (discontinuous) brought together from linearly distant regions of the polypeptide chain by the folding of that chain as it exists in the native state, (see Atassi, M. Z., Immunochemistry 15, 909-936 (1978)). As in the case of lysozyme several of the elements consist of only one amino acid, the size of a contributing element can then vary between one and the maximum number of amino acids consistent with the dimensions of the antibody combining site, and is likely to be of the order of five to six, (see Atassi, M. Z., supra).

The precise localization of immunogenic determinants within the amino acid sequence of a few proteins has been performed by one or more of the following approaches: (1) antigenicity measurements of the whole polypeptide or peptide fragments isolated therefrom, before and after chemical modification at specific residues; (2) locating the position, within the polypeptide amino acid sequence of substitutions, selected by growing the virus expressing the protein in the presence of monoclonal antibodies; and (3) synthesis and testing of peptides, homologous with the amino acid sequence, of regions suspected of immunogenic activity.

U.S. Pat. No. 4,554,101 discloses a method of determining the antigenic or allergenic determinants of protein antigens or allergens on the basis of the determination of the point of greatest local average hydrophilicity of such protein antigens or allergens. Furthermore, the patent teaches generating a synthetic peptide containing a designated sequence of six or more amino acids corresponding to the point of greatest local average hydrophilicity.

Using methods known to the skilled artisan such as those described in U.S. Pat. No. 4,554,101, the antigenic peptides for the various protein antigens or allergens could be obtained and incorporated into a trans-body. For example, antigenic peptides could be obtained from Hepatitis B surface antigen, histocompatibility antigens, influenza hemaglutinin, fowl plague virus hemagglutinin, rag weed allergens Ra3 and Ra5 and the antigens of the following viruses: vaccinia, Epstein-Barr virus, polio, rubella, cytomegalovirus, small pox, herpes, simplex types I and II, yellow fever, and many others.

Additionally, antigenic peptides could be obtained from the following parasites: organisms carrying malaria (P. Falciporum, P. Ovace, etc.). Schistosomiasis, Onchocerca Volvulus and other filiarial parasites, Tyrpanosomes, Leishmania, Chagas disease, amoebiasis, hookworm, and the like. In addition, antigenic peptides could be obtained from the following bacteria: leprosy, tuberculosis, syphilis, gonorrhea and the like.

Further, using known methods, antigenic peptides could be obtained from the following viruses: Infectious ectromelia virus, Cowpox virus, Herpes simples virus, Infectious bovine rhinotracheitis virus, Equine rhinopneumonitis (equine abortion) virus, Malignant catarrh virus of cattle, Feline rhinotracheitis virus, Canine herpesvirus, Epstein-Barr virus (ass. with infectious mononucleosis and Burkitt lymphoma), Marek's disease virus, Sheep pulmonary adenomatosis (Jaagziekle) virus, Cytomegaloviruses, Adenovirus group, Human papilloma virus, Feline panleucopaenia virus, Mink enteritis virus, African horse sickness virus (9 serotypes), Blue tongue virus (12 serotypes), Infectious pancreatic necrosis virus of trout, Fowl sarcoma virus (various strains), Avian leukosis virus, visceral, Avian leukosis virus, erythroblastic, Avian leukosis virus, myeloblastic, Osteopetrosis virus, Newcastle disease virus, Parainfluenza virus 1, Parainfluenza virus 2. Parainfluenza virus 3, Parainfluenza virus 4, Mumps virus, Turkey virus, CANADA/58, Canine distemper virus, Measles virus, Respiratory syncytial virus, Myxovirus, Type A viruses such as Human influenza viruses, e.g. Ao/PR8/34, A1/CAM/46, and A2/Singapore/1/57; Fowl plague virus; Type B viruses e.g. B/Lee/40; Rabies virus; Eastern equinine encephalitis virus; Venezuelan equine encephalitis virus; Western equine encephalitis virus; Yellow fever virus, Dengue type 1 virus (type 6), Dengue type 2 virus (type 5); Dengue type 3 virus; Dengue type 4 virus; Japanese encephalitis virus, Kyasanur Forest virus; Louping i11 virus, Murray Valley encephalitis virus; Omsk haemorrhagic fever virus (types 1 and 11); St. Louis encephalitis virus; Human rhinoviruses, Foot-and-mouth disease virus; Poliovirus type 1; Enterovirus Polio 2; Enterovirus Polio 3; Avian infectious bronchitis virus; Human respiratory virus; Transmissible gastro-enteritis virus of swine; Lymphocytic choriomeningitis virus, Lassa virus; Machupo virus; Pichinde virus; Tacaribe virus; Papillomavirus.

In one aspect, the trans-bodies of the present invention comprise antigenic peptides selected from proteins that have already been used for vaccines, such as proteins from polio and rubella. In another aspect, the trans-bodies of the present invention comprise antigenic peptides that are known to be suitable for vaccination.

U.S. Pat. Nos. 4,694,071 and 4,857,634 describe synthetic peptides suitable for vaccinations against a disease caused by an enterovirus. These peptides are derived from the structural capsid protein VP1 for poliovirus type 3 Sabin strain.

U.S. Pat. No. 4,708,871 discloses synthetic peptides that display the antigenicity of the VP1 protein of foot-and-mouth disease virus, characterized in that at least a portion of the peptide is selected from the group consisting of five, six, or seven antigenically active amino acid sequence of a VP1 protein.

U.S. Pat. No. 4,769,237 provides synthetic peptides useful for generating antibodies that protect animal hosts from picornaviruses. Specifically, the patent teaches antigenic peptides containing a sequence of about 20 amino acid residues corresponding to a certain region of the antigenic picornavirus capsid protein, such as the VP1 capsids of foot-and-mouth disease and poliomyelitis viruses.

U.S. Pat. No. 4,474,757 teaches synthetic peptides for generating vaccines against various influenza strains. The antigenic fragments are derived from the specific determinants of several influenza strains and in the hemagglutinin of several influenza strains.

U.S. Pat. No. 5,427,792 discloses linear and cyclic peptides of the E1 and E2 glycoproteins of the rubella virus, and U.S. Pat. No. 5,164,481 discloses linear and cyclic peptides of the E1 and C proteins of rubella virus. These peptides are also useful in the manufacture of vaccines against rubella viral infections. U.S. Pat. Nos. 6,180,758 and 6,037,44 disclose synthetic peptides having an amino acid sequence corresponding to at least one antigenic determinant of a structural protein, particularly the E1, E2 or C protein, of rubella virus (RV), for use in vaccines against rubella.

U.S. Pat. No. 5,866,694 provides peptides that induce antibodies which neutralize genetically divergent HIV-1 isolates. The peptides are six amino acids in length and are derived from gp 160.

U.S. Pat. No. 4,777,239 discloses seventeen synthetic peptides which are capable of raising antibodies specific for certain desired human papilloma virus (HPV). The peptides are selected on the basis of predicted secondary structure and hydrophilicity from proteins or peptides encoded by selected open reading frames. The secondary structure and hydrophilicity are deduced from the amino acid sequence of these proteins according to methods disclosed by Hopp, T., et al., Proc Natl Acad Sci (USA) (1981) 78: 3824; Levitt, M., J Mol Biol (1976) 104: 59; and Chou, P., et al., Biochem (1974) 13: 211. The results of these deductions permit the construction of peptides which elicit antibodies reactive with the entire protein. Two general types of such antigenic peptides are prepared. Peptide regions identified as being specific to HPV-16 or other HPV type-specific determinants by lack of homology with other HPV types lead to the peptides which are useful to raise antibodies for diagnostic, protective, and therapeutic purposes against HPV-16 or other virus type per se. Peptide regions which are homologous among the various types of HPV of interest are useful as broad spectrum diagnostics and vaccines, and elicit antibodies that are broad spectrum diagnostics.

U.S. Pat. No. 6,410,720 discloses peptide antigens derived from Mycobacterium vaccae useful for treating mycobacterial infections including Mycobacterium tuberculosis and Mycobacterium avium. The soluble antigen induces an immune response in patients previously exposed to a mycobacterium.

U.S. Pat. No. 6,488,931 provides vaccines comprising polypeptides containing an immunogenic portion of an ovarian carcinoma protein and peptide variants thereof that differ in one or more substitutions, deletions, additions and/or insertions such that the ability of the variant to react with ovarian carcinoma protein-specific antisera is not substantially diminished.

U.S. Pat. No. 6,489,101 discloses polypeptides comprising at least a portion of a breast tumor protein, or a variant thereof that are immunogenic for generating vaccines useful for the treatment and prevention of breast cancer.

U.S. Pat. No. 6,447,778 teaches peptides conjugates for generating vaccines that induce cell mediated immune response by stimulating the production and proliferation of cytotoxic lymphocytes. The peptide conjugates comprise amino acid sequences similar to the gp120 principal neutralizing domain (PND) of HIV, gp41, and Nef (p27) of HIV and carriers which enhance immunogenicity.

U.S. Pat. No. 6,419,931 provides peptides for inducing a cytotoxic T lymphocyte (CTL) response to an antigen of interest in a mammal. Typically the CTL inducing peptide will be from seven to fifteen residues, and more usually from nine to eleven residues. The CTL inducing peptides which are useful in the compositions and methods of the present invention can be selected from a variety of sources, depending of course on the targeted antigen of interest. The CTL inducing peptides are typically small peptides that are derived from selected epitopic regions of target antigens associated with an effective CTL response to the disease of interest.

U.S. Pat. No. 6,419,931 is also directed to a composition comprising the CTL inducing peptide and a peptide capable of eliciting a helper T lymphocyte (HTL) response. HTL-inducing epitopes can be provided by peptides which correspond substantially to the antigen targeted by the CTL-inducing peptide, or is a peptide to a more widely recognized antigen, and is not specific for a particular histocompatibility antigen restriction. Peptides which are recognized by most individuals regardless of their MHC class II phenotype (“promiscuous”) may be particularly advantageous. The HTL peptide will typically comprise from six to thirty amino acids and contain a HTL-inducing epitope.

CTL responses are an important component of the immune responses of most mammals to a wide variety of viruses. U.S. Pat. No. 6,419,931 provides a means to effectively stimulate a CTL response to virus-infected cells and treat or prevent such an infection in a host mammal. Thus, the compositions and methods of the patent are applicable to any virus presenting protein and/or peptide antigens. Such viruses include but are not limited to the following, pathogenic viruses such as influenza A and B viruses (FLU-A, FLU-B), human immunodeficiency type I and II viruses (HIV-I, HIV-II), Epstein-Barr virus (EBV), human T lymphotropic (or T-cell leukemia) virus type I and type II (HTLV-I, HTLV-II), human papillomaviruses types 1 to 18 (HPV-1 to HPV-18), rubella (RV), varicella-zoster (VZV), hepatitis B (HBV), hepatitis C (HCV), adenoviruses (AV), and herpes simplex viruses(HV). In addition, the patent is applicable to peptide antigens of cytomegalovirus (CMV), poliovirus, respiratory syncytial (RSV), rhinovirus, rabies, mumps, rotavirus and measles viruses.

In a like manner, the compositions and methods of U.S. Pat. No. 6,419,931 are applicable to tumor-associated proteins, which could be sources for CTL epitopes. Such tumor proteins and/or peptides, include, but are not limited to, products of the MAGE-1, -2 and -3 genes, products of the c-ErbB2 (HER-2/neu) proto-oncogene, tumor suppressor and regulatory genes which could be either mutated or overexpressed such as p53, ras, myc, and R3 1. Tissue specific proteins to target CTL responses to tumors such as prostatic specific antigen (PSA) and prostatic acid phosphatase (PAP) for prostate cancer, and tyrosinase for melanoma. In addition viral related proteins associated with cell transformation into tumor cells such as EBNA-1, HPV E6 and E7 are likewise applicable. A large number of peptides from some of the above proteins have been identified for the presence of MHC-binding motifs and for their ability to bind with high efficiency to purified MHC molecules.

U.S. Pat. No. 6,407,063 discloses specific antigenic peptides of MAGE-1 and MAGE-4 that can be used to make vaccines to elicit immune responses for treating diseases.

U.S. Pat. Nos. 5,462,871; 5,558,995; 5,554,724; 5,585,461; 5,591,430; 5,554,506; 5,487,974; 5,530,096; and 5,519,117 disclose peptides that elicit specific T cell responses (either CD4⁺ or CD8⁺ T cells), such as tumor-associated antigenic peptides (TAA, also known as TRAS for tumor rejection antigens). See also review by Van den Eynde and van der Bruggen (1997) and Shawler et al. (1997).

U.S. Pat. No. 6,368,852 disclose a peptide capable of inducing CTL (Cytotoxic T Lymphocytes) to human gastric cells in vivo or in vitro. More specifically, the peptide is capable of presenting CTL to human gastric cells by being bound to HLA-A31 antigen (Human Leucocyte Antigen). The peptides may be used for producing a vaccine for treating and preventing gastric cancer.

Peptides from the Fc Region

Immunoglobulins (Igs) are produced by B lymphocytes and secreted into plasma. The Ig molecule in monomeric form is a glycoprotein with a molecular weight of approximately 150 kDa that is shaped more or less like a Y. As discussed earlier, the Y shape is composed of two heavy chains and two light chains. The heavy chain is divided into an Fc portion, which is at the carboxyl terminal (the base of the Y), and a Fab portion, which is at the amino terminal (the arm of the Y). Carbohydrate chains are attached to the Fc portion of the molecule. The Fc portion of the Ig molecule is composed only of heavy chains. Fc regions of IgG and IgM can bind to receptors on the surface of immunomodulatory cells such as macrophages and stimulate the release of cytokines that regulate the immune response. The Fc region contains protein sequences common to all Igs as well as determinants unique to the individual classes. These regions are referred to as the constant regions because they do not vary significantly among different Ig molecules within the same class. The constant region of the Fc fragment confers the biological properties of the molecule, e.g. binding to receptors and activation of complement.

Fc receptors are activated by the binding of the active sites within the Fc region. Fc receptors are, therefore, the critical link between antibodies and the remainder of the immune system. Fc receptor binding to antibody Fc region active sites may thus be characterized as the “final common pathway” by which antibody functions are mediated. If an antigen-bound antibody does not bind to an Fc receptor, the antibody is unable to activate the other portions of the immune system and is therefore rendered functionally inactive.

Any peptide with the ability to bind to immunoglobulin Fc receptors has therapeutic usefulness as an immunoregulator by virtue of the peptide's ability to regulate binding to the receptor. Such an Fc receptor “blocker” occupies the immunoglobulin binding site of the Fc receptor and thus “short circuits” the immunoglobulin's activating ability.

The present invention provides trans-bodies comprising peptides derived from the Fc region of immunoglobulins for regulating the immune response. The present invention contemplates the use of such trans-bodies for both therapeutic and diagnostic purposes associated with modulating the immune response. The peptides inserted into a trans-body can stimulate an immune response by binding to the Fc receptor or inhibit an immune response by blocking the binding to the Fc receptor.

U.S. Pat. No. 4,816,449 discloses sequences of new and useful peptides that are capable of reducing inflammatory responses associated with autoimmune diseases, allergies and other inflammatory conditions such as those mediated by the mammalian immune system. In particular, the claimed pentapeptides are useful in blocking inflammation mediated by the arachadonic acid/leukotriene-prostaglandin pathway. Thus, the peptides may be used effectively in the place of known anti-inflammatory agents, such as steroids, many of which exhibit harmful or toxic side effects. Although these peptides bear a structural similarity to the Cε3 aa 320-324 portion of human IgE, thought to be associated with IgE Fc receptor binding, it is thought that the present mechanism of anti-inflammatory activity surprisingly does not necessarily involve blocking of Fc receptor binding. Rather, the present peptides have been shown to interact directly in the arachadonic acid-mediated inflammatory pathway and thereby reduce such inflammation. It is believed, however, that the morphological similarities between the present peptides and the IgE molecule may render the claimed peptides useful in regulating immune system-mediated responses, as for example by acting as Fc receptor site blockers. The claimed peptides have an amino acid sequence A-B-C-D-E (SEQ ID NO: 5), wherein

A is Asp or Glu;

B is Ser, D-Ser, Thr, Ala, Gly or Sarcosine;

C is Asp, Glu, Asn or Gln;

D is Pro, Val, Ala, Leu or Ile; and

E is Arg, Lys or Orn.

U.S. Pat. No. 4,753,927 describes the sequences of new and useful peptides that can block the binding of human IgG immune complexes to IgG Fc receptors on human polymorphonuclear neutrophils (PMNs), of IgG and IgE immune complexes to IgG and IgE Fc receptors on monocytes and macrophages (MMs) and other white blood cells. The patent provides a method of modulating immune responses in mammals by blocking immune complex binding to immunoglobulin Fc receptors comprising administering a peptide comprising a portion selected from the amino acid sequence -Pro-Asp-Ala-Arg-His-Ser-Thr-Thr-Gln-Pro-Arg- (SEQ ID NO: 6). The patent also teaches the use of the peptides for reducing human allergic reaction for reducing immune complex mediated inflammation and tissue destruction.

Depending upon the particular type of Fc receptor to which an active site peptide binds, the peptide may either stimulate or inhibit immune functions. Stimulation may occur if the Fc receptor is of the type that becomes activated by the act of binding to an Fe region or, alternatively, if an Fc active site peptide stimulates the receptor. The type of stimulation produced may include, but is not limited to, functions directly or indirectly mediated by antibody Fc region-Fc receptor binding. Examples of such functions include, but are not limited to, stimulation of phagocytosis by certain classes of white blood cells (polymorphonuclear neutrophils, monocytes and macrophages; macrophage activation, antibody-dependent cell mediated cytotoxicity (ADCC); natural killer (NK) cell activity; growth and development of B and T lymphocytes and secretion by lymphocytes of lymphokines (molecules with killing or immunoregulatory activities).

The present invention contemplates the use of trans-bodies comprising peptides that interact with the Fc Receptor and stimulate immune system functions, including those listed above. These trans-bodies are therapeutically useful in treating diseases such as infectious diseases caused by bacteria, viruses or fungi, conditions in which the immune system is deficient due either to congenital or acquired conditions, cancer and many other afflictions of human beings or animals. Such immunostimulation is also useful to boost the body's protective cellular and antibody response to certain injected or orally administered substances administered as vaccines. This list merely provides representative examples of diseases or conditions in which immune stimulation has established therapeutic usefulness.

Inhibition of immune system functions may occur if an active site peptide binds to a particular Fc receptor which is not activated by the mere act of binding to an Fe region. Such Fc receptors normally become “activated” only when several Fc regions within an antigen-antibody aggregate or immune complex simultaneously bind to several Fc receptors, causing them to become “crosslinked”. Such Fc receptor crosslinking by several Fc regions appears to be the critical signal required to activate certain types of Fe receptors. By binding to and blocking such an Fc receptor, an active site peptide will prevent Fc regions within immune complexes or antigen-antibody aggregates from binding to the receptor, thus blocking Fc receptor activation.

The present invention contemplates the use of trans-bodies comprising peptides that interact with the Fc receptor to inhibit immune system functions. Such trans-bodies are therapeutically useful in treating diseases such as allergies, autoimmune diseases including rheumatoid arthritis and systemic lupus erythematosis, certain types of kidney diseases, inflammatory bowel diseases such as ulcerative colitis and regional enteritis (Crohn's disease), certain types of inflammatory lung diseases such as idiopathic pulmonary fibrosis and hypersensitivity pneumonitis, certain types of demylinating neurologic diseases such as multiple sclerosis, autoimmune hemolytic anemias, idiopathic (autoimmune) thrombocytopenic purpura, certain types of endocrinological diseases such as Grave's disease or Hashimoto's thyroiditis and certain types of cardiac disease such as rheumatic fever. Immunosuppression is also therapeutically useful in preventing the harmful immune “rejection” response which occurs with organ transplantation or in transplantation of bone marrow cells used to treat certain leukemias or aplastic anemias. This list merely provides representative examples of diseases or conditions in which immunosuppression is known to be therapeutically useful.

Johnson and Thames (J. Immunol., 117, 1491 (1975)) and Boackle, Johnson and Caughman (Nature, 282, 742 (1979)) found that peptides with sequences derived from the C_{H}2 of human IgG1 at aa (amino acids) 274-281 (Lys-Phe-Asn-Trp-Tyr-Val-Asp-Gly, SEQ ID NO: 7) had substantial complement activating ability when the peptides were adsorbed to erythrocytes. In particular, one peptide with the aa (amino acid sequence) (Lys-Ala-Asp-Trp-Tyr-Val-Asp-Gly, SEQ ID NO: 8) was about as effective in activating C1q-mediated cell lysis as immune complexes formed by heat aggregated IgG. The aforementioned researchers attributed this activity to the peptide's ability to act as an active binding site for the C1q Fc receptor. Other synthetic peptides with sequences derived from this region of IgG or from the aa 487-491 region of C_{H}4 of IgM (Glu-Trp-Met-Gln-Arg, SEQ ID NO: 9).

Subsequently, Prystowsky, et al. (Biochemistry, 20, 6349 (1981)), and Lukas, et al. (J. Immunol., 127, 2555 (1981)) demonstrated that peptides from an immediately adjacent C_{H}2 region from aa281 to 292 were inhibitors of C1-mediated hemolysis. Specifically, peptides identical to IgG, C_{H}2 residues 281-290 (Gly-Val-Gln-Val-His-Asn-Ala-Lys-Thr-Lys, SEQ ID NO: 10) and aa 282-292 (Val-Gln-Val-His-Asn-Ala-Lys-Thr-Lys-Pro-Arg-OH, SEQ ID NO: 11) were approximately as active as inhibitors as intact monomeric IgG. Other peptides, such as aa 275-290 (Phe-Asn-Trp-Tyr-Val-Asp-Gly-Val-Gln-Val-His-Asn-Ala-Lys-Thr-Lys, SEQ ID NO: 12), and aa 275-279 (Ac-Phe-Asn-Trp-Tyr-Val, SEQ ID NO: 13), aa 289-292 (Thr-Lys-Pro-Arg, SEQ ID NO: 14) were less active.

Tuftsin is a tetrapeptide, with sequence Thr-Lys-Pro-Arg (SEQ ID NO: 14), and is present in the second constant domain of all human IgG subclasses and in guinea pig IgG at aa 289-292. U.S. Pat. No. 3,778,426 shows that it stimulates phagocytosis by granulocytes, monocytes and macrophages in vitro and is described in. Additionally, Tuftsin has been shown to stimulate ADCC, Natural Killer (NK) cell activity, macrophage-dependent-T-cell education and antibody synthesis to T-cell-dependent and independent antigens in vitro and in vivo. Studies by Ratcliffe and Stanworth (Immunol. Lett., 4, 215 (1982)) demonstrate that Tuftsin does bind to IgG Fc receptors since it competitively inhibits human IgG binding to human monocyte IgG Fc receptors.

Morgan et al (Proc. Natl. Acad. Sci. USA, 79, 5388 (1982)) disclose the sequence of a 24 residue peptide identical to IgG aa 335-358 with the ability to nonspecifically activate lymphocytes. The peptide was shown to induce polyclonal B cell proliferation, antigen-specific antibody responses and Natural Killer (NK) cell-mediated lysis. This peptide (Thr-Ile-Ser-Lys-Ala-Lys-Gly-Gln-Pro-Arg-Glu-Pro-Gln-Val-Tyr-Thr-Leu-Pro-Ser-Arg-Glu-Glu-Met, SEQ ID NO: 15) and the 23 residue peptide lacking the carboxy-terminal methionine probably acts by binding to lymphocyte Fc receptors for IgG.

Ciccimarra, et al (Proc. Natl. Acad. Sci. USA, 72, 2081 (1975)) report the sequence of a decapeptide from human IgG which could block IgG binding to human monocyte IgG Fc receptors. This peptide is identical to IgG aa 407-416 (Tyr-Ser-Lys-Leu-Thr-Val-Asp-Lys-Ser-Arg, SEQ ID NO: 16).

Ratcliffe and Stanworth (Immunol. Lett., 4,215 (1982)) show that a peptide identical to IgG aa 295-301 (Gln-Tyr-Asp-Ser-Thr-Tyr-Arg, SEQ ID NO: 17) could slightly block IgG binding to human monocyte IgG Fc receptors. By contrast, a related peptide identical to IgG, C_{H}2, residues at aa 289-301 had no monocyte IgG blocking activity.

Hamburger describes that a pentapeptide with sequence derived from human IgE C.sub..epsilon. 3 at aa 320-324 (Asp-Ser-Asp-Pro-Arg, SEQ ID NO: 18) could inhibit a local cutaneous allergic reaction (Prausnitz-Kustner) by approximately 90% (Hamburger, Science, 189, 389 (1975) and U.S. Pat. Nos. 4,171,299 and 4,161,322). This peptide has subsequently been shown to inhibit systemic allergic disease in humans after injection by the subcutaneous route. Studies demonstrate that the peptide has significant affinity for the IgE Fc receptor of human basophils and can block human IgE binding to basophil IgE Fc receptors by up to 70% (Plummer, et al., Fed. Proc., 42, 713 (1983)). The observed ability of this peptide to block systemic allergic disease in humans is attributed to the peptide's ability to bind to cellular IgE Fc receptors (Hamburgr, Adv. Allergology Immunol. (Pergamon Press: New York, 1980), pp. 591-593).

Hamburger reports that a hexapeptide derived from Cε4 at aa 476-481 (Pro-Asp-Ala-Arg-His-Ser, SEQ ID NO: 19) could block block IgE-binding to IgE Fc receptors on a human lymphoblastoid cell line (wil-2wt) (Hamburger, Immunology, 38, 781 (1979)). This peptide had been previously implicated as an agent useful in blocking IgE-binding to human basophil IgE Fc receptors (U.S. Pat. No. 4,161,522).

Stanworth (Mol. Immunol., 19, 1245 (1982)) describes that a decapeptide with sequence identical to a portion of Cε4 of human IgE at aa 505-515 (Val-Phe-Ser-Arg-Leu-Glu-Val-Thr-Arg-Ala-Glu, SEQ ID NO: 20) caused a marked enhancement of binding of ¹²⁵I-human IgG to mouse macrophages.

Stanworth, et al. demonstrated that certain peptides with sequences identical to portions of Cε4 of human IgE, aa 495-506 (Pro-Arg-Lys-Thr-Lys-Gly-Ser-Gly-Phe-Phe-Val-Phe, SEQ ID NO: 21) and smaller derivatives thereof were able to cause degranulation of human and rodent mast cells and thus might be useful in allergic desensitization therapy. (Biochem, J., 180, 665 (1979); Biochem, J., 181, 623 (1979); and European Patent Publication EP 0000252).

Sarmay et al. (Mol. Immunol., 1988, 25(11):1183-8) summarize the results showing the effect of synthetic peptides composed of surface exposed residues of Cγ2 or Cγ3 domains on different steps of human B lymphocyte activation cycle. Both the C_{H}2 (289Thr-301Arg) and C_{H}3 (407Tyr-416Arg) peptides as well as the whole Fc fragment enhanced the IgM synthesis of PWM or PMA+CAI activated lymphocytes. This effect was exerted at the early phase of B cell activation. The incubation of separated resting B cells with Fc fragments or C_{H}2 peptides resulted in increase of cell volume and in expression of HLA-DR antigen. On the other hand, LIF production was induced both by C_{H}2 and C_{H}3 peptides. It was also shown that Fc peptides induce IL-1 release from monocytes. The results suggest that the C_{H}2 and C_{H}3 domain peptides exert their effect partly directly, by activating resting B cells, rendering the cells more susceptible to other stimuli; and moreover, by enhancing the humoral response by triggering the release of IL-1.

Sheridan et al. (J Pept Sci 1999, 5(12):555-62) teaches solid phase synthesis of a large branched disulphide peptide from IgG Fc, Ac-F-C*-A-K-V-N-N-K-D-L-P-A-P-I-E-K(Ac-E-L-L-G-G-P-S-V-F)-C*-I-NH2. This peptide combines the lower hinge region of IgG and a proximal beta-hairpin loop, both implicated in binding to FcγRI. Cyclic hinge-loop peptide was active in displacing IgG2a from FcγRI expressed on monocyte cell lines with an IC50 of 40 microM, whereas the linear form of this peptide was inactive. The Fc hinge-loop peptide demonstrates the potential for a non-mAb high affinity, immunomodulatory ligand for FcγRI.

Methods of Using Transferrin/TNF-SCA Trans-Bodies

In one aspect, the present invention provides trans-bodies comprising one or more antibody variable region or CDRs of tumor necrosis factor-alpha (TNF-α) antibodies and transferrin or modified transferrin. The present invention contemplates the use of such trans-bodies for therapeutic and diagnostic purposes. Examples of serious disease states related to the production of TNF-α includes, but are not limited to, the following: septic shock; endotoxic shock; cachexia syndromes associated with bacterial infections (e.g., tuberculosis, meningitis), viral infections (eg., AIDS), parasite infections (e.g., malaria), and neoplastic disease; autoimmune disease, including some forms of arthritis (especially rheumatoid and degenerative forms); and adverse effects associated with treatment for the prevention of graft rejection. As discussed below, TNF-α is associated with various diseases states or conditions. The present invention contemplates the use of the anti-TNF trans-bodies for the treatment and diagnosis of a variety of diseases.

TNF-α

TNF-α is a pleiotropic inflammatory cytokine. Most organs of the body appear to be affected by TNF-α. This cytokine possesses both growth stimulatory as well as growth inhibitory properties. It also appears to have self regulatory properties. For example, TNF-α induces neutrophil proliferation during inflammation, but it also induces neutrophil apoptosis upon binding to the TNF-R55 receptor (Murray et al., 1997, Blood, 90(7): 2772-2783). The cytokine is produced by several types of cells, but especially macrophages. Although the role of cytokines in pathophysiological states has not been fully elucidated, TNF-α appears to be a major mediator in the cascade of injury and morbidity.

Although many factors contribute to the inflammatory response, TNF-α plays the major role in regulating this process. The cellular effects of TNF-α include physiologic, cytotoxic, and inflammatory processes. In homeostasis, TNF-α influences mitogenesis, differentiation, and immunoregulation while causing apoptotic cell death in neoplastic cell lines. Cytotoxicity by TNF-α occurs independently of de novo transcription and translation and involves mitochondrial production of oxygen radicals generated primarily at the ubisemiquinone site.

The biologic effects of TNF-α depend on its concentration and site of production: at low concentrations, TNF-α may produce desirable homeostatic and defense functions, but at high concentrations, systemically or in certain tissues, TNF-α can synergize with other cytokines, notably interleukin-1 (IL-1) to aggravate many inflammatory responses.

The following activities have been shown to be induced by TNF-α (together with IL-1); fever, slow-wave sleep, hemodynamic shock, increased production of acute phase proteins, decreased production of albumin, activation of vascular endothelial cells, increased expression of major histocompatibility complex (MHC) molecules, decreased lipoprotein lipase, decreased cytochrome P450, decreased plasma zinc and iron, fibroblast proliferation, increased synovial cell collagenase, increased cyclo-oxygenase activity, activation of T cells and B cells, and induction of secretion of the cytokines, TNF-α itself, IL-1, IL-6, and IL-8. Indeed, studies have shown that the physiological effects of these cytokines are interrelated (Philip et al., Nature (1986) 323(6083):86-89; Wallach., D. et al., J. Immunol. (1988) 140(9):2994-2999). Though the detail as to how TNF-α exerts its effects is not known, many of the effects are thought to be related to the ability of TNF-α to stimulate cells to produce prostaglandins and leukotrienes from arachidonic acid of the cell membrane.

TNF-α, as a result of its pleiotropic effects, has been implicated in a variety of pathologic states in many different organs of the body. In blood vessels, TNF-α promotes hemorrhagic shock, down regulates endothelial cell thrombomodulin and enhances a procoagulant activity. It causes the adhesion of white blood cells and probably of platelets to the walls of blood vessels, and so, may promote processes leading to atherosclerosis, as well as to vasculitis.

TNF-α activates blood cells and causes the adhesion of neutrophils, eosinophils, monocytes/macrophages, and T and B lymphocytes. By inducing IL-6 and IL-8, TNF-α augments the chemotaxis of inflammatory cells and their penetration into tissues. Thus, TNF-α has a role in the tissue damage of autoimmune diseases, allergies and graft rejection.

TNF-α has also been called cachectin because it modulates the metabolic activities of adipocytes and contributes to the wasting and cachexia accompanying cancer, chronic infections, chronic heart failure, and chronic inflammation. Cachexia is the extensive wasting which is associated with cancer, and other diseases (Kern, et al. J. Parent. Enter. Nutr. 12: 286-298 (1988)). Cachexia includes progressive weight loss, anorexia, and persistent erosion of body mass in response to a malignant growth. The fundamental physiological derangement can relate to a decline in food intake relative to energy expenditure. The cachectic state causes most cancer morbidity and mortality. TNF-α can mediate cachexia in cancer, infectious pathology, and other catabolic states. TNF-α may also have a role in anorexia nervosa by inhibiting appetite while enhancing wasting of fatty tissue.

TNF-α has metabolic effects on skeletal and cardiac muscle. It has also marked effects on the liver: it depresses albumin and cytochrome P450 metabolism and increases production of fibrinogen, 1-acid glycoprotein and other acute phase proteins. It can also cause necrosis of the bowel.

In the central nervous system, TNF-α crosses the blood-brain barrier and induces fever, increased sleep and anorexia. Increased TNF-α concentration is associated with multiple sclerosis. It further causes adrenal hemorrhage and affects production of steroid hormones, enhances collagenase and PGE-2 in the skin, and causes the breakdown of bone-and cartilage by activating osteoclasts.

TNF-α has been shown to facilitate and augment human immunodeficiency virus (HIV) replication in vitro (Matsuyama, T. et al., J. Virol. (1989) 63(6):2504-2509; Michihiko, S. et al., Lancet (1989) 1(8648):1206-1207) and to stimulate HIV-1 gene expression, thus, probably triggering the development of clinical AIDS in individuals latently infected with HIV-1 (Okamoto, T. et al., AIDS Res. Hum. Retroviruses (1989) 5(2):131-138).

TNF-α has also been shown to be involved in the control of growth and differentiation of various parasites. Upon infection of the host, parasites are capable of inducing the secretion of different cytokines such as TNF which may affect the course of the disease. For instance, in the case of malaria, TNF-α can be protective in certain circumstances, such as inhibiting parasite survival in rodent malaria (Clark et al., 1987, J Immunol 139:3493-3496.; Taverne et al., 1987, Clin Exp Immunol 67:1-4).

TNF-α Antibodies

Any CDR, V_{H} or V_{L} region from an antibody that binds to TNF may be used to make trans-bodies of the invention. Polyclonal murine antibodies to TNF are disclosed by Cerami et al. (EPO Patent Publication 0212489, Mar. 4, 1987). Such antibodies were said to be useful in diagnostic immunoassays and in therapy of shock in bacterial infections. Rubin et al. (EPO Patent Publication 0218868, Apr. 22, 1987) disclose murine monoclonal antibodies to human TNF, the hybridomas secreting such antibodies, methods of producing such murine antibodies, and the use of such murine antibodies in immunoassay of TNF.

Yone et al. (EPO Patent Publication 0288088, Oct. 26, 1988) discloses anti-TNF murine antibodies, including mAbs, and their utility in immunoassay diagnosis of pathologies, in particular Kawasaki's pathology and bacterial infection. The body fluids of patients with Kawasaki's pathology (infantile acute febrile mucocutaneous lymph node syndrome; Kawasaki, Allergy 16: 178 (1967); Kawasaki, Shonica (Pediatrics) 26: 935 (1985)) were said to contain elevated TNF levels which were related to progress of the pathology (Yone et al., infra).

Other investigators have described rodent or murine mAbs specific for recombinant human TNF which had neutralizing activity in vitro (Liang et al., Biochem. Biophys. Res. Comm. 137: 847-854 (1986); Meager et al., Hybridoma 6: 305-311 (1987); Fendly et al., Hybridoma 6: 359-369 (1987); Bringman et al., Hybridoma 6: 489-507 (1987); Hirai et al., J. Immunol. Meth. 96: 57-62 (1987); Moller et al. Cytokine 2: 162-169 (1990)). Some of these mAbs were used to map epitopes of human TNF and develop enzyme immunoassays (Fendly et al., infra; Hirai et al., infra; Moller et al., infra) and to assist in the purification of recombinant TNF (Bringman et al, infra).

Neutralizing antisera or mAbs to TNF have been shown in mammals other than man to abrogate adverse physiological changes and prevent death after lethal challenge in experimental endotoxemia and bacteremia. This effect has been demonstrated, e.g., in rodent lethality assays and in primate pathology model systems (Mathison et al., J. Clin. Invest. 81: 1925-1937 (1988); Beutler et al., Science 229: 869-871 (1985); Tracey et al., Nature 330: 662-664 (1987); Shimamoto et al., Immunol. Lett. 17: 311-318 (1988); Silva, et al., J. Infect. Dis. 162: 421-427 (1990); Opal et al., J. Infect. Dis. 161: 1148-1152 (1990); Hinshaw et al., Circ. Shock 30: 279-292 (1990)).

Putative receptor binding loci of hTNF has been disclosed by Eck and Sprang (J. Biol. Chem. 264 (29), 17595-17605 (1989), who identified the receptor binding loci of TNF-α as consisting of amino acids 11-13, 37-42, 49-57 and 155-157.

Administration of murine TNF mAb to patients suffering from severe graft versus host pathology has also been reported (Herve et al., Lymphoma Res. 9: 591 (1990)).

U.S. Pat. No. 5,656,272 discloses anti-TNF antibodies, fragments and regions thereof which are specific for human TNF-α and are useful in vivo for diagnosis and therapy of a number of TNF-α mediated pathologies and conditions such as Crohn's disease.

U.S. Pat. No. 6,420,346 discloses a method of treating rheumatoid arthritis of an individual, the method comprising intramuscularly administering an exogenous polynucleotide encoding an immunogenic portion of a cytokine such as TNF-α, operatively linked to a promoter, wherein the expression of said immunogenic portion induces a formation of antibodies to said immunogenic portion, wherein said antibodies reduce an in vivo activity of an endogenous cytokine of-said cytokines, to thereby treat rheumatoid arthritis.

Maini et al. describes the use of infliximab, a chimeric TNF-α monoclonal antibody, for treating patients with rheumatoid arthritis (Lancet, 354(9194):1932-9 (1999)).

Kits Containing Trans-Bodies

In a further embodiment, the present invention provides kits containing transferrin fusion proteins, preferably trans-bodies and modified trans-bodies comprising immunomodulatory peptides, which can be used, for instance, for the therapeutic, non-therapeutic, or diagnostic applications. The kit comprises a container with a label. Suitable containers include, for example, bottles, vials, and test tubes. The containers may be formed from a variety of materials such as glass or plastic. The container holds a composition which includes a transferrin fusion protein, preferably a trans-body, that is effective for therapeutic or non-therapeutic applications, such as described above. The active agent in the composition is the antibody. The label on the container indicates that the composition is used for a specific therapy or non-therapeutic application, and may also indicate directions for either in vivo or in vitro use, such as those described above.

The kit of the invention will typically comprise the container described above and one or more other containers comprising materials desirable from a commercial and user standpoint, including buffers, diluents, filters, needles, syringes, and package inserts with instructions for use.

Without further description, it is believed that a person of ordinary skill in the art can, using the preceding description and the following illustrative examples, make and utilize the present invention and practice the claimed methods. For example, a skilled artisan would readily be able to determine the biological activity, both in vitro and in vivo, for the fusion protein constructs of the present invention as compared with the comparable activity of the therapeutic moiety in its unfused state. Similarly, a person skilled in the art could readily determine the serum half life and serum stability of constructs according to the present invention. The following working examples therefore, specifically point out the preferred embodiments of the present invention, and are not to be construed as limiting in any way the remainder of the disclosure.

### EXAMPLES

The following examples describe methods for generating trans-bodies comprising peptides that bind target proteins and transferrin or modified transferrin (mTf). The fusion of a therapeutic peptide (X) such as a single chain antibody or an antigen binding peptide at the N- or C-termini of transferrin (X-Tf-X) with or without the use of a linker (L) or linkers (X-Tf-L-X, X-L-Tf-X, X-L-Tf-L-X), will allow for development of a bivalent drug.

This facilitates construction of a targeted molecule, for example fusion of a single chain antibody and a toxic peptide at each end of the Transferrin molecule. A typical application would be targeted killing of cancer cells. Also, a SCA at both the N- and C-termini could provide a bifunctional antibody with Transferrin acting as an Fc hinge. This would provide a cost effective technology for replacing (humanized) monoclonal antibody technology.

As discussed earlier, there are a number of loops within the Transferrin protein structure that may be amenable to modification/replacement for the insertion of proteins or peptides and the development of a screenable library of random peptide inserts.

### Example 1

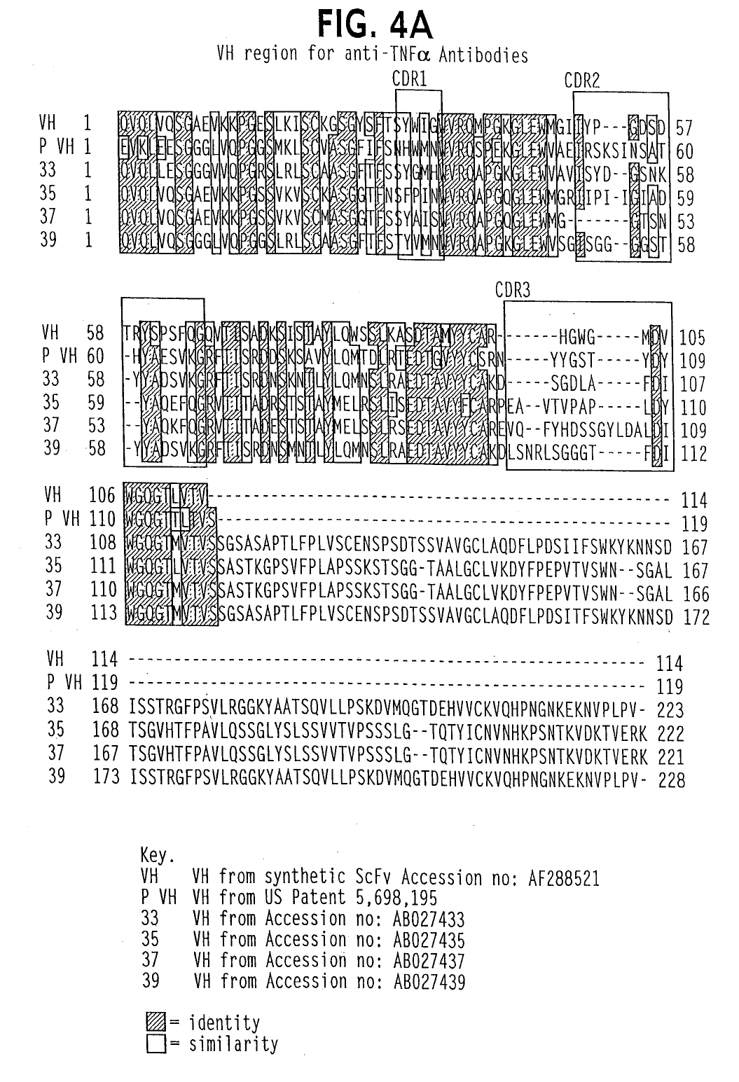

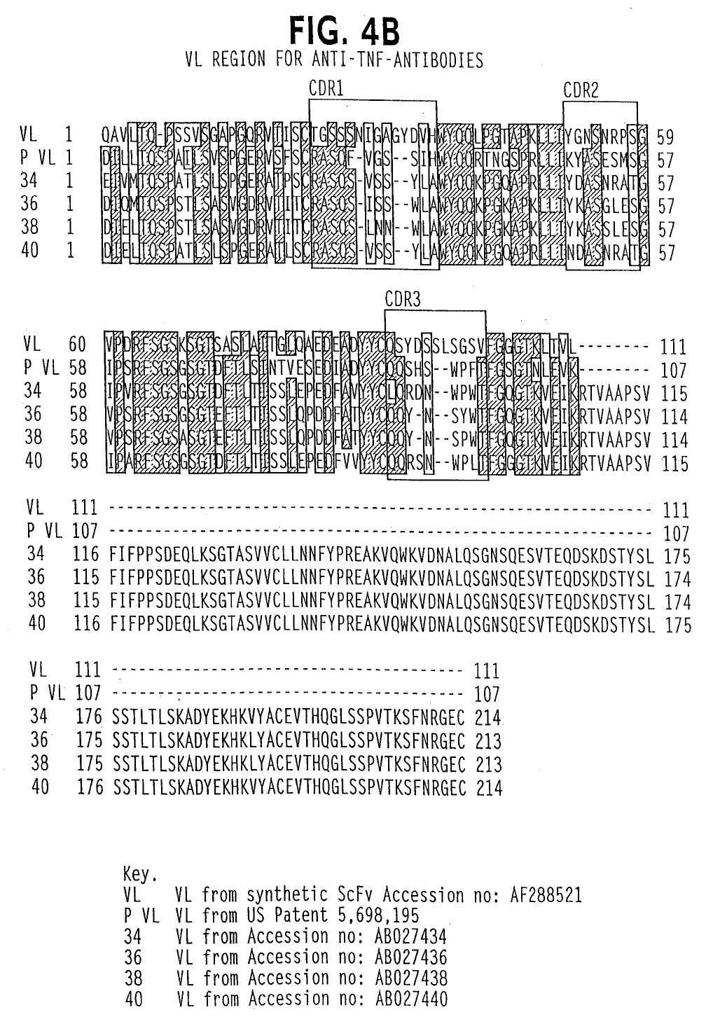

A trans-body comprising a transferrin molecule and a single chain antibody can be produced. A specific example of a SCA that can be fused to transferrin is anti-TNF (tumor necrosis factor). Anti-TNF has been used to treat various inflammatory and autoimmune diseases. TNF-SCA could be fused to the N- or C-terminus of modified transferrin in such manner that the coding N-terminus of TNF-SCA is directly attached to the C-terminal amino acid of Transferrin or the C-terminal amino acid of TNF-SCA is directly attached to the N-terminal amino acid of Transferrin. Alternatively, a peptide linker could be inserted to provide more separation between Transferrin and TNF-SCA and allow more spatial mobility to the two fused proteins. Several examples of TNF-SCA are shown in FIGS. 4A-4B.

A fusion protein between modified Tf (mTf) and TNF-SCA is made by fusing one or more copies of the nucleotide sequence encoding the SCA to the nucleotide sequence of Tf to produce a fusion protein with a SCA fused to the N- or C-terminus of Tf. A vector containing the nucleic acid encoding mTf, such as pREX5004, is specifically designed for generating mTf fusion proteins with V_{H}, V_{L}, or CDRs. Linkers and primers are specifically designed for ligating the sequences encoding V_{H}, V_{L}, or CDRs into vectors containing mTf.

Construction of Anti-TNFα SCA mTf N- and C-terminal Fusions.

The first step in this process is to insert into pREX5004 a linker between the XbaI and KpnI sites at the 5′, or N-terminus, of mTf into which the V_{H} and V_{L} could subsequently be cloned to generate pREX5057. This linker contains sites for the insertion of the V_{H} and V_{L} at either end of a DNA linker coding for, in this example, an S (SGG)₃ S (SEQ ID NO: 24) linker peptide.

```
XbaI/Sacl-linker-EcoRV/KpnI insert
                                                      SacI
                                                    -----+
ctagataaaa gggaagtgaa actggagctc tggtggtggt tctggtggtg gttctggtgg
        tatttt cccttcactt tgacctcgag accaccacca agaccaccac caagaccacc
                                                          >>...............SG Linker.........
    . . . . . . . . . s g g g s g g g s g
                EcoRV
              ----+--
tggttctgat atcaacctgg aagtgaaggt ac
accaagacta tagttggacc ttcacttc
. . . >>
g g s d i n l e v k v
Top strand: SEQ ID NO: 22
Bottom Strand: SEQ ID NO: 23
Amino Acid Sequence: SEQ ID NO: 24
```

The DNA for the V_{H} and V_{L} is then generated, separately, using a series of overlapping synthetic oligonucleotides. The V_{H} is designed with a 5′ XbaI site and a 3′ SacI site and is inserted into pREX5057 cut with XbaI//SacI. The correct insertion and DNA sequence of the insert is confirmed and the resulting plasmid named pREX5058. The V_{L} is designed with a 5′ EcoRV site and 3′ KpnI site and is inserted into pREX5058 cut with EcoRV/KpnI. The correct insertion and DNA sequence of the insert is confirmed and the resulting plasmid named pREX5059.

Using a pair of mutagenic PCR primers, the 5′ and 3′ ends of the completed SCA in pREX5059 are then modified such that the resulting product could be inserted at the C-terminus of mTf (pREX5004) via SalI and HindIII sites. The correct insertion and DNA sequence of the insert was confirmed and the resulting plasmid named pREX5060.

Forward: AGCCTGCACTTTCCGTCGACCTGAAGTGAAACTGGAAG (5′ to 3′) SEQ ID NO: 25
Reverse: CAGTCATGTCTAAGCTTATTACTTCACTTCCAGGTTGG (5′ to 3′) SEQ ID NO: 26

The expression cassettes from pREX5059 and pREX5060 were recovered by PsiI/AgeI digestion and inserted into PsiI/AgeI cut yeast vector, such as pSAC3, to produce pREX5061 and pREX5062. These were used for transformation and expression in yeast.

To make a V_{H}-mTf-V_{L} fusion construct the V_{H} in pREX5058 is modified at the 3′ end to insert a KpnI site. The V_{L} in pREX5059 is modified at the 5′ to introduce a SalI site. The modified V_{H} and V_{L} are then inserted sequentially into the 5′ and 3′ ends of mTf (pREX5004), the V_{H} at the N-terminus via the XbaI and KpnI sites (pREX5063) and the V_{L} at the C-terminus via SalI and HindIII sites (pREX5064). The expression cassette from this vector is then sub-cloned via PsiI/AgeI sites into a yeast vector, such as pSAC3, to generate pREX5065.

Alternatively the V_{L} could be at the N-terminus and the V_{H} at the C-terminus. Additionally the V_{H} or V_{L} alone could be at the N-terminus or the V_{H} or V_{L} alone could be at the C-terminus. Variations on this theme also include use of the S (SGG)₃ S (SEQ ID NO: 24) linker peptide between the V_{H} or V_{L} and N- or C-termini. Also a construct with the V_{H}/V_{L} at both the N- and C-termini could be constructed in which the V_{H}/V_{L} are identical or against different targets. Similarly, the single V_{H} or V_{L} at the N- and C-termini could be against different targets.

| anti-TNF alpha | Translation product | 240 amino acids |
|---|---|---|
| | Mol. Wt. | 25964.1 |
| | Isoelectric point (pI) | 5.77 |

| | |
|---|---|
| EVKLEESGGGLVQPGGSMKLSCVASGFIFSNHWMNWVRQSPEKGLEWVAEIRSK | (SEQ ID NO: 27) |
| SINSATHYAESVKGRFTISRDDSKSAVYLQMTDLRTEDTGVYYCSRNYYGSTYDY | |
| WGQGTTLTVSSSGGGSGGGSGGGSDILLTQSPAILSVSPGERVSFSCRASQFVGSS | |
| IHWYQQRTNGSPRLLIKYASESMSGIPSRFSGSGSGTDFTLSINTVESEDIADYYCQ | |
| QSHSWPFTFGSGTNLEVK | |
| V_{H} DNA Sequence | |

| 1 | gaagtgaaac tggaagaaag cggcggcggc ctggtgcagc cgggcggcag catgaaactg |
|---|---|
| | cttcactttg accttctttc gccgccgccg gaccacgtcg gcccgccgtc gtactttgac |
| | >>.......................anti TNFalpha VH........................> |
| | e v k l e e s g g g l v q p g g s m k l |
| 61 | agctgcgtgg cgagcggctt tatttttagc aaccattgga tgaactgggt gcgtcagagc |
| | tcgacgcacc gctcgccgaa ataaaaatcg ttggtaacct acttgaccca cgcagtctcg |
| | >.......................anti TNFalpha VH........................> |
| | s c v a s g f i f s n h w m n w v r q s |
| 121 | ccggaaaaag gcctggaatg ggtggcggaa attcgtagca aaagcattaa cagcgcgacc |
| | ggcctttttc cggaccttac ccaccgcctt taagcatcgt tttcgtaatt gtcgcgctgg |
| | >.......................anti TNFalpha VH........................> |
| | p e k g l e w v a e i r s k s i n s a t |
| 181 | cattatgcgg aaagcgtgaa aggccgtttt accattagcc gtgatgatag caaaagcgcg |
| | gtaatacgcc tttcgcactt tccggcaaaa tggtaatcgg cactactatc gttttcgcgc |
| | >.......................anti TNFalpha VH........................> |
| | h y a e s v k g r f t i s r d d s k s a |
| | >..........CDR2.........>> |
| | PstI |
| | ------+ |
| 241 | gtgtatctgc agatgaccga tctgcgtacc gaagataccg gcgtgtatta ttgcagccgt |
| | cacatagacg tctactggct agacgcatgg cttctatggc cgcacataat aacgtcggca |
| | >.......................anti TNFalpha VH........................> |
| | v y l q m t d l r t e d t g v y y c s r |
| 301 | aactattatg gcagcaccta tgattattgg ggccagggca ccaccctgac cgtgagc |
| | ttgataatac cgtcgtggat actaataacc ccggtcccgt ggtgggactg gcactcg |
| | >.......................anti TNFalpha VH.......................>> |
| | n y y g s t y d y w g q g t t l t v s |
| | >>..........CDR3...........>> |
| V_{H} DNA Sequence = SEQ ID NO: 28 | |
| anti TNFalpha V_{H} sequence = SEQ ID NO: 29 | |
| V_{L} DNA Sequence | |
| 1 | gatattctgc tgacccagag cccggcgatt ctgagcgtga gcccgggcga acgtgtgagc |
| | ctataagacg actgggtctc gggccgctaa gactcgcact cgggcccgct tgcacactcg |
| | >>.......................anti TNFalpha........................> |
| | d i l l t q s p a i l s v s p g e r v s |
| 61 | tttagctgcc gtgcgagcca gtttgtgggc agcagcattc attggtatca gcagcgtacc |
| | aaatcgacgg cacgctcggt caaacacccg tcgtcgtaag taaccatagt cgtcgcatgg |
| | >.......................anti TNFalpha........................> |
| | f s c r a s q f v g s s i h w y q q r t |
| | >>..............CDR1...............>> |
| 121 | aacggcagcc cgcgtctgct gattaaatat gcgagcgaaa gcatgagcgg cattccgagc |
| | ttgccgtcgg gcgcagacga ctaatttata cgctcgcttt cgtactcgcc gtaaggctcg |
| | >.......................anti TNFalpha........................> |
| | n g s p r l l i k y a s e s m s g i p s |
| | >>.........CDR2.........>> |
| 181 | cgttttagcg gcagcggcag cggcaccgat tttaccctga gcattaacac cgtggaaagc |
| | gcaaaatcgc cgtcgccgtc gccgtggcta aaatgggact cgtaattgtg gcacctttcg |
| | >.......................anti TNFalpha........................> |
| | r f s g s g s g t d f t l s i n t v e s |
| 241 | gaagatattg cggattatta ttgccagcag agccatagct ggccgtttac ctttggcagc |
| | cttctataac gcctaataat aacggtcgtc tcggtatcga ccggcaaatg gaaaccgtcg |
| | >.......................anti TNFalpha........................> |
| | e d i a d y y c q q s h s w p f t f g s |
| | >>...........CDR3...........>> |
| 301 | ggcaccaacc tggaagtgaa a |
| | ccgtggttgg accttcactt t |
| | >....anti TNFalpha...>> |
| | g t n l e v k |

V₁ DNA Sequence = SEQ ID NO: 30
anti TNFalpha V₁ sequence = SEQ ID NO: 31
Peptide Linker
Ser (Ser Gly Gly Gly)₃ Ser (SEQ ID NO: 32)
tct (tct ggt ggt ggt)₃ tct (SEQ ID NO: 33)
tcttctg gtggtggttc tggtggtggt tctggtggtg gttct (SEQ ID NO: 33)
agaagac caccaccaag accaccacca agaccaccac caaga (SEQ ID NO: 34)
s s g g g s g g g s g g g s (SEQ ID NO: 32)

### Example 2

A trans-body comprising transferrin and CDRs may be generated. These usually consist of relatively short stretches of peptides. Antibodies normally have three CDRs in their heavy chains and three in their light chains. One or more CDRs of an antibody which can interact with the antigen can be fused to modified transferrin to confer antigen binding activity to Transferrin molecule. The CDRs can be fused to the N-, C-, N- and C-termini or engineered into the interior scaffold of transferrin. Examples of the CDRs sequences from anti-TNF antibodies are shown in the TNF-SCA FIGS. 4A-4B. cDNAs corresponding to one or more CDRs can be fused with modified transferrin to confer TNF binding activity to transferrin.

Insertion of CDR(s)

Examination of the N-domain of human Tf (PDB identifier 1A8E) and the full Tf model AAAaoTfwo, generated using the ExPasy Swiss Model Server with the rabbit model 1JNF as template, reveals a number of potential sites for insertion of a peptide, either directly or by replacement of a number of residues. These sites are duplicated by their equivalent sites in the C domain.

| N₁ | N₂ |
|---|---|
| Asp33 | Ser105 |
| Asn55 | Glu141 |
| Asn75 | Asp166 |
| Asp90 | Gln184 |
| Gly257 | Asp197 |
| Lys280 | Lys217 |
| His289 | Thr231 |
| Ser298 | Cys241 |

Two of these loops are sites into which a CDR peptide, particularly CDR, H3 is inserted, N₁ His289 (286-292) or N₂ Asp166 (162-170). Due to the structural similarity between the N and C domain the equivalent insertion sites on the C domain (C₁ 489-495, C₂ 623-628) are also used to make the molecule multivalent. This is done using a variety of the potential insert sites indicated above either on just the N or C domain or by a combination of sites on both domains.

| | | |
|---|---|---|
| N₂ | 140 | PEPRKPLEKA VANFFSGSCA PC [symbol] CQLCP---- -GCGCSTLNQ |
| C₁ | 472 | NH------CR FDEFFSEGCA PG [symbol] CKLCMGSGL NLCEPNNKEG |
| N₂ | 140 | PEP [symbol] KPLEKA VAN [symbol] GS [symbol] [symbol] C [symbol] [symbol] P [symbol] C [symbol] - -- [symbol] STL [symbol] --Q |
| C₁ | 472 | NHC [symbol] ------ FDE [symbol] EG [symbol] [symbol] G [symbol] [symbol] M [symbol] S [symbol] L NL [symbol] EPN [symbol] KEG |
| N₁ | 277 | D-KSKE--FQ LF [symbol] L LFKDSAHGFL KVPPRMDAKM YLGYEYVTAI |
| C₂ | 611 | NVTDCSGNFC LF [symbol] L LFRDDTVCLA KLHDRNTYEK YLGEEYVKAV |
| N₁ | 277 | --- [symbol] K [symbol] KE [symbol] Q [symbol] [symbol] [symbol] [symbol] [symbol] K [symbol] SAHGFL [symbol] PP [symbol] MDA [symbol] M [symbol] Y [symbol] T [symbol] |
| C₂ | 611 | NVT [symbol] C [symbol] GN [symbol] C [symbol] [symbol] [symbol] [symbol] [symbol] R [symbol] DTVCLA [symbol] HD [symbol] NTY+Z 818 K [symbol] E [symbol] K [symbol] |

N₂: SEQ ID NO:35
C₁: SEQ ID NO:36
N₁: SEQ ID NO:37
C₂: SEQ ID NO:38

Examination of sequences for several SCA against the antigen TNFα available from Genbank yielded the following CDR's (Table 3). Any one of these peptides is useful as a binding peptide (see for example, Misawa et al., 2002, FEBS Lett. 525: 77; Steinbergs et al., 1996, Hum. Antibodies Hybridomas, 7(3): 106; Jarrin et al., 1994, FEBS Lett. 354: 169). However, as linear peptides, the binding affinities are generally lower than that of the antibody from which they originated. By inserting the peptide(s) into the scaffold of another protein some or all of this affinity can be recovered. With mTf as the scaffold the possibility of insertion at multiple site, possibly in combination with other CDRs from the same origin exists.

TABLE 3

| | | |
|---|---|---|
| CDR1 | CDR2 | CDR3 |

| | | | |
|---|---|---|---|
| VH | [symbol] | I [symbol] YP--- [symbol] D [symbol] DTR [symbol] [symbol] PSF [symbol] [symbol] | -------HGWG-----M [symbol] V |
| | SEQ ID NO:39 | SEQ ID NO:40 | SEQ ID NO:41 |
| P VH | [symbol] | E [symbol] RSKSINS [symbol] -H [symbol] ESVK z,811 | N-----YYGST-----Y [symbol] Y |
| | SEQ ID NO:42 | SEQ ID NO:43 | SEQ ID NO:44 |
| 33 | [symbol] | V [symbol] SYD-- [symbol] SNK-Y [symbol] DSVK [symbol] | D-----SGDLA-----F [symbol] I |
| | SEQ ID NO:45 | SEQ ID NO:46 | SEQ ID NO:47 |
| 35 | [symbol] | R [symbol] IPI-I [symbol] IAD- [symbol] QEFQ [symbol] | PEA-VTVPAP-----L [symbol] Y |
| | SEQ ID NO:48 | SEQ ID NO:49 | SEQ ID NO:50 |
| 37 | [symbol] | ------- [symbol] TSN- [symbol] QKFQ [symbol] | EVQ-FYHDSSGYLDAL [symbol] I |
| | SEQ ID NO:51 | SEQ ID NO:52 | SEQ ID NO:53 |
| 39 | [symbol] | G [symbol] SGG-- [symbol] GST-Y [symbol] DSVK [symbol] | DLSNRLSGGGT-----F [symbol] I |
| | SEQ ID NO:54 | SEQ ID NO:55 | SEQ ID NO:56 |
| VL | TG [symbol] GYD [symbol] H | YGN [symbol] NRP | [symbol] SYD [symbol] SLSGSV |
| | SEQ ID NO:57 | SEQ ID NO:58 | SEQ ID NO:59 |
| P VL | [symbol] F- [symbol] G [symbol] -S [symbol] H | KY [symbol] ESM | [symbol] SH [symbol] -WPF [symbol] |
| | SEQ ID NO:60 | SEQ ID NO:61 | SEQ ID NO:62 |
| 33 | [symbol] - [symbol] S [symbol] -Y [symbol] A | YD [symbol] NRA | L [symbol] RD [symbol] -WPW [symbol] |
| | SEQ ID NO:63 | SEQ ID NO:64 | SEQ ID NO:65 |
| 35 | [symbol] - [symbol] S [symbol] -W [symbol] A | YK [symbol] GLE | [symbol] Y- [symbol] -SYW [symbol] |
| | SEQ ID NO:66 | SEQ ID NO:67 | SEQ ID NO:68 |
| 37 | [symbol] - [symbol] N [symbol] -W [symbol] A | YK [symbol] SLE | [symbol] Y- [symbol] -SPW [symbol] |
| | SEQ ID NO:69 | SEQ ID NO:70 | SEQ ID NO:71 |
| 39 | [symbol] - [symbol] S [symbol] -Y [symbol] A | ND [symbol] NRA | [symbol] RS [symbol] -WPL [symbol] |
| | SEQ ID NO:72 | SEQ ID NO:73 | SEQ ID NO:74 |

Key.
V_{H} VH from synthetic ScFv Accession no: AF288521
P V_{H} VH from US Pat. 5,698,195
33 VH from Accession no: AB027433
35 VH from Accession no: AB027435
37 VH from Accession no: AB027437
39 VH from Accession no: AB027439
Dark gray = identity
Light gray = similarity

As an example CDR3 from P VH above is inserted into the N domain of mTF between Asp166 and Phe167. The sequence is back translated into DNA using codons optimized for yeast expression.

| | |
|---|---|
| aat tat tat ggt tct act tat gat tat | (SEQ ID NO: 80) |
| N Y Y G S T Y D Y | (SEQ ID NO: 44) |

Using pREX5004 as a template and the mutagenic primer P0109 with primer P0025, and mutagenic primer P0110 with primer P0012, two PCR products are generated. These are subsequently joined together using the external primers P0025 and P0012. This results in the insertion of CDR H3 between Asp166 and Phe167. The PCR product from this joining reaction is then digested with BamHI and EcoRI and inserted back into pREX5004 also digested with BamHI/EcoRI. The expression cassettes from the resulting plasmid, pREX5079, is then recovered by PsiI/AgeI digestion and inserted into PsiI/AgeI cut yeast vector, such as pSAC3, to produce pREX5080 and transformed into yeast for protein expression.

| | |
|---|---|
| | BamHI |
| | -+---- |
| 541 | agcctgtggt ggcagagttc tatgggtcaa aagaggatcc acagactttc tattatgctg |
| | tcggacacca ccgtctcaag atacccagtt ttctcctagg tgtctgaaag ataatacgac |
| | >..............................mTf...............................> |
| | k p v v a e f y g s k e d p q t f y y a |
| 601 | ttgctgtggt gaagaaggat agtggcttcc agatgaacca gcttcgaggc aagaagtcct |
| | aacgacacca cttcttccta tcaccgaagg tctacttggt cgaagctccg ttcttcagga |
| | >..............................mTf...............................> |
| | v a v v k k d s g f q m s q l r g k k s |
| 661 | gccacacggg tctaggcagg tccgctgggt ggaacatccc cataggctta ctttactgtg |
| | cggtgtgccc agatccgtcc aggcgaccca ccttgtaggg gtatccgaat gaaatgacac |
| | >..............................mTf...............................> |
| | c h t g l g r s a g w n i p i g l l y c |
| | acttacctga gccacgtaaa cctcttgaga aagcagtggc caatttcttc tcgggcagct |
| | tgaatggact cggtgcattt ggagaactct ttcgtcaccg gttaaagaag agcccgtcga |
| | >..............................mTf...............................> |
| | d l p e p r k p l e k a v a n f f s g s |
| | P0110 |
| 781 | gtgccccttg tgcggatggg acgaattatt atggttctac ttatgattat gacttccccc |
| | cacggggaac acgcctaccc tgcttaataa taccaagatg aatactaata ctgaaggggg |
| | P0109 |
| | >..............................mTf...............................> |
| | c a p c a d g t n y y g s t y d y d f p |
| | >>.....................162-170......................> |
| | a d g t n y y g s t y d y d f p |
| | n y y g s t y d y |
| 841 | agctgtgtca actgtgtcca gggtgtggct gctccaccct taaccaatac ttcggctact |
| | tcgacacagt tgacacaggt cccacaccga cgaggtggga attggttatg aagccgatga |
| | >..............................mTf...............................> |
| | q l c q l c p g c g c s t l s q y f g y |
| | > >>162-170 |
| | /q//l |
| 901 | cgggagcctt caagtgtctg aaggatggtg ctggggatgt ggcctttgtc aagcactcga |
| | gccctcggaa gttcacagac ttcctaccac gacccctaca ccggaaacag ttcgtgagct |
| | >..............................mTf...............................> |
| | s q a f k c l k d g a g d v a f v k h s |
| 961 | ctatatttga gaacttggca aacaaggctg acagggacca gtatgagctg ctttgcctgg |
| | gatataaact cttgaaccgt ttgttccgac tgtccctggt catactcgac gaaacggacc |
| | >..............................mTf...............................> |
| | t i f e n l a n k a d r d q y e l l c l |
| 1021 | acaacacccg gaagccggta gatgaataca aggactgcca cttggcccag gtcccttctc |
| | tgttgtgggc cttcggccat ctacttatgt tcctgacggt gaaccgggtc cagggaagag |
| | >..............................mTf...............................> |
| | d n t r k p v d e y k d c h l a q v p s |
| 1081 | ataccgtcgt ggcccgaagt atgggcggca aggaggactt gatctgggag cttctcaacc |
| | tatggcagca ccgggcttca tacccgccgt tcctcctgaa ctagaccctc gaagagttgg |
| | >..............................mTf...............................> |
| | h t v v a r s m g g k e d l i w e l l n |
| | EcoRI |
| | _+----- |
| 1141 | aggcccagga acattttggc aaagacaaat caaaagaatt ccaactattc agctctcctc |
| | tccgggtcct tgtaaaaccg tttctgttta gttttcttaa ggttgataag tcgagaggag |
| | >..............................mTf...............................> |
| | q a q e h f g k d k s k e f q l f s s p |

| | |
|---|---|
| Top Stand: | SEQ ID NO: 75 |
| Peptide Strand: | SEQ ID NO: 76 |
| Amino acids 162-170: | SEQ ID NO: 77 |
| CDR H3: | SEQ ID NO: 44 |
| P0109 | |
| ATAATCATAAGTAGAACCATAATAATTCGTCCCATCCGCACAAGGGGCACAGC | (SEQ ID NO: 78) |
| TGC | |
| P0110 | |
| GAATTATTATGGTTCTACTTATGATTATGACTTCCCCCAGCTGTGTCAACTG | (SEQ ID NO: 79) |

### Example 3

The trans-bodies in Examples 1 and 2 can be further modified to include an antigenic or immunomodulatory peptide. The desired peptide can be inserted in the transferrin portion of the trans-body. In this way, the modified trans-body not only can bind their antigens, but can also induce an immune response in the host.

Although the present invention has been described in detail with reference to examples above, it is understood that various modifications can be made without departing from spirit of the invention. Accordingly, the invention is limited only by the following claims. All cited patents, patent applications and publications referred to in this application are herein incorporated by reference in their entirety.

We claim:

1. A fusion protein comprising a transferrin (Tf) protein exhibiting reduced glycosylation fused to at least one antibody variable region.

2. A fusion protein of claim 1, wherein the antibody variable region comprises a V_{H}, V_{L}, or a CDR region.

3. A fusion protein of claim 1, comprising at least two antibody variable regions.

4. A fusion protein of claim 3, comprising at least a V_{H} and V_{L} region, a V_{H} and V_{H} region, or a V_{L} and V_{L} region.

5. A fusion protein of claim 1, comprising at least two different antibody variable regions.

6. A fusion protein of claim 5, wherein the different antibody variable regions specifically bind different antigens.

7. A fusion protein of claim 3, wherein the fusion protein is engineered so that the antibody variable regions are in close proximity.

8. A fusion protein of claim 7, wherein the antibody variable regions are inserted into two adjacent Tf loops.

9. A fusion protein of claim 8, wherein one antibody variable region is fused to the C-terminus of Tf and one antibody variable region is inserted into an adjacent Tf loop.

10. A fusion protein of claim 8, wherein one antibody variable region is fused to the N-terminal end of Tf and one antibody variable region is inserted into an adjacent Tf loop.

11. A fusion protein of claim 9, wherein the Tf C-terminal proline residue is deleted.

12. A fusion protein of claim 9, wherein the Tf C-terminal cysteine loop is deleted.

13. A fusion protein of claim 8, wherein the antibody variable regions are fused to the N- and C-terminal ends of Tf.

14. A fusion protein of claim 1, wherein the antibody variable region comprises at least one CDR peptide.

15. A fusion protein of claim 14, wherein the CDR peptide specifically binds an antigen.

16. A fusion protein of claim 14, wherein the CDR peptide is derived from an antibody.

17. A fusion protein of claim 14, wherein the CDR peptide is from a peptide library.

18. A fusion protein of claim 1, wherein the antibody variable region specifically binds to tumor necrosis factor (TNF).

19. A fusion protein of claim 14, wherein the CDR specifically binds to TNF.

20. A fusion protein of claim 1, wherein the at least one antibody variable region comprises amino terminal domains of a V_{H} or V_{L} region of an antibody.

21. A fusion protein of claim 20, wherein the amino terminal domain comprises at least one CDR.

22. A fusion protein of claim 21, wherein the amino terminal domain comprises 3 CDRs.

23. A fusion protein of claim 1, wherein the antibody variable region is fused to the C-terminal end of Tf.

24. A fusion protein of claim 1, wherein the antibody variable region is fused to the N-terminal end of Tf.

25. A fusion protein of claim 1, wherein the antibody variable region is inserted into at least one loop of the Tf.

26. A fusion protein of claim 1, wherein the Tf protein has reduced affinity for a transferrin receptor (TfR).

27. The fusion protein of claims 1, wherein the Tf protein is lacto transferrin (lactoferrin).

28. A fusion protein of claim 26, wherein the Tf protein does not bind a TfR.

29. A fusion protein of claim 1, wherein the Tf protein has reduced affinity for iron.

30. A fusion protein of claim 29, wherein the Tf protein does not bind iron.

31. A fusion protein of claim 1, wherein said Tf protein comprises at least one mutation that prevents glycosylation.

32. A fusion protein of claim 31, wherein the Tf protein is lacto transferrin (lactoferrin).

33. A fusion protein of claim 1, which is expressed in the presence of tunicamycin

34. A fusion protein of claim 1, wherein said Tf protein comprises a portion of the N domain of a Tf protein, a bridging peptide and a portion of the C domain of a Tf protein.

35. A fusion protein of claim 34, wherein the bridging peptide links the antibody variable region to Tf

36. A fusion protein of claim 34, wherein the antibody variable region is inserted between an N and a C domain of Tf protein.

37. A fusion protein of claim 1, wherein the Tf protein comprises at least one amino acid substitution, deletion or addition in the hinge region.

38. A fusion protein of claim 37, wherein said hinge region is selected from the group consisting of about residue 94 to about residue 96, about residue 245 to about residue 247, about residue 316 to about residue 318, about residue 425 to about residue 427, about residue 581 to about residue 582 and about residue 652 to about residue 658.

39. A fusion protein of claim 1, wherein said Tf protein has at least one amino acid substitution, deletion or addition at a position selected from the group consisting of Asp 63, Gly 65, Tyr 95, Tyr 188, Lys 206, His 207, His 249, Asp 392, Tyr 426, Tyr 514, Tyr 517, His 585, Thr 120, Arg 124, Ala 126, Gly 127, Thr 452, Arg 456, Ala 458 and Gly 459.

40. A fusion protein of claim 25, wherein the antibody variable region replaces at least one loop of Tf.

41. A fusion protein of claim 31, wherein the glycosylation site is selected from the group consisting of an amino acid residue corresponding to amino acids N413, N611.

42. A fusion protein of claim 26 or 28, wherein the Tf comprises at least one amino acid substitution, deletion or addition at an amino acid residue corresponding to an amino acid selected from the group consisting of Asp 63, Gly 65, Tyr 95, Tyr 188, Lys 206, His 207, His 249, Asp 392, Tyr 426, Tyr 514, Tyr 517, His 585, Thr 120, Arg 124, Ala 126, Gly 127, Thr 452, Arg 456, Ala 458 and Gly 459.

43. A fusion protein comprising a transferrin (Tf) protein exhibiting reduced affinity for a transferrin receptor (TfR) fused to at least one antibody variable region.

44. A fusion protein of claim 43, comprising at least two antibody variable regions.

45. A fusion protein of claim 44, comprising at least a V_{H} and V_{L} region, a V_{H} and V_{H} region, or a V_{L} and V_{L} region.

46. A fusion protein of claim 43, comprising at least two different antibody variable regions.

47. A fusion protein of claim 46, wherein the different antibody variable regions specifically bind different antigens.

48. A fusion protein of claim 44, wherein the fusion protein is engineered so that the antibody variable regions are in close proximity.

49. A fusion protein of claim 48, wherein the antibody variable regions are inserted into two adjacent Tf loops.

50. A fusion protein of claim 49, wherein one antibody variable region is fused to the C-terminus of Tf and one antibody variable region is inserted into an adjacent Tf loop.

51. A fusion protein of claim 49, wherein one antibody variable region is fused to the N-terminal end of Tf and one antibody variable region is inserted into an adjacent Tf loop.

52. A fusion protein of claim 50, wherein the Tf C-terminal proline residue is deleted.

53. A fusion protein of claim 50, wherein the Tf C-terminal cysteine loop is deleted.

54. A fusion protein of claim 49, wherein the antibody variable regions are fused to the N- and C-terminal ends of Tf.

55. A fusion protein of claim 43, wherein the antibody variable region comprises at least one CDR peptide.

56. A fusion protein of claim 55, wherein the CDR peptide specifically binds an antigen.

57. A fusion protein of claim 55, wherein the CDR peptide is derived from an antibody. (???)

58. A fusion protein of claim 55, wherein the CDR peptide is from a peptide library.

59. A fusion protein of claim 43, wherein the antibody variable region specifically binds to tumor necrosis factor (TNF).

60. A fusion protein of claim 55, wherein the CDR specifically binds to TNF.

61. A fusion protein of claim 43, wherein the antibody variable region comprises amino terminal domains of a V_{H} or V_{L} region of an antibody.

62. A fusion protein of claim 61, wherein the amino terminal domain comprises at least one CDR.

63. A fusion protein of claim 62, wherein the amino terminal domain comprises 3 CDRs.

64. A fusion protein of claim 1 or 43, wherein the serum half-life of the antibody variable region is increased over the serum half-life of the antibody variable region in an unfused state.

65. A fusion protein of claim 43, wherein the therapeutic protein or peptide is fused to the C-terminal end of Tf.

66. A fusion protein of claim 43, wherein the therapeutic protein or peptide is fused to the N-terminal end of Tf.

67. A fusion protein of claim 43, wherein the therapeutic protein or peptide is inserted into at least one loop of the Tf.

68. A fusion protein of claim 43, wherein the TF protein does not bind a TfR.

69. A fusion protein of claim 43, wherein the Tf protein has reduced affinity for iron.

70. A fusion protein of claim 69, wherein the Tf protein does not bind iron.

71. A fusion protein of claim 43, wherein said Tf protein exhibits reduced or no glycosylation.

72. A fusion protein of claim 71, comprising at least one mutation that prevents glycosylation.

73. A fusion protein of claim 43, wherein said Tf protein comprises a portion of the N domain of a Tf protein, a bridging peptide and a portion of the C domain of a Tf protein.

74. A fusion protein of claim 73, wherein the bridging peptide links the therapeutic protein or peptide to Tf.

75. A fusion protein of claim 73, wherein said therapeutic protein, peptide or polypeptide is inserted between an N and a C domain of Tf protein.

76. A fusion protein of claim 43, wherein the Tf protein have at least one amino acid substitution, deletion or addition in the Tf hinge region.

77. A fusion protein of claim 76, wherein said hinge region is selected from the group consisting of about residue 94 to about residue 96, about residue 245 to about residue 247, about residue 316 to about residue 318, about residue 425 to about residue 427, about residue 581 to about residue 582 and about residue 652 to about residue 658.

78. A fusion protein of claim 43, wherein said Tf protein has at least one amino acid substitution, deletion or addition at a position selected from the group consisting of Asp 63, Gly 65, Tyr 95, Tyr 188, Lys 206, His 207, His 249, Asp 392, Tyr 426, Tyr 514, Tyr 517, His 585, Thr 120, Arg 124, Ala 126, Gly 127, Thr 452, Arg 456, Ala 458 and Gly 459.

79. A fusion protein of claim 67, wherein the therapeutic protein or peptide replaces at least one loop.

80. A fusion protein of claim 71, wherein the glycosylation site is selected from the group consisting of an amino acid residue corresponding to amino acids N413, N611.

81. A nucleic acid molecule encoding a fusion protein of either claim 1 or 43.

82. A vector comprising a nucleic acid molecule of claim 81.

83. A host cell comprising a vector of claim 82.

84. A host cell comprising a nucleic acid molecule of claim 81.

85. A method of expressing a Tf fusion protein comprising culturing a host cell of claim 83 under conditions which express the encoded fusion protein.

86. A method of expressing a Tf fusion protein comprising culturing a host cell of claim 84 under conditions which express the encoded fusion protein.

87. A host cell of claim 83, wherein the cell is prokaryotic or eukaryotic.

88. A host cell of claim 84, wherein the cell is prokaryotic or eukaryotic.

89. A host cell of claim 87, wherein the cell is a yeast cell.

90. A host cell of claim 88, wherein the cell is a yeast cell.

91. A transgenic animal comprising a nucleic acid molecule of 81.

92. A method of producing a Tf fusion protein comprising isolating a fusion protein from a transgenic animal of claim 91.

93. A method of claim 92, wherein the Tf fusion protein comprises lactoferrin.

94. A method of claim 93, wherein the fusion protein is isolated from a biological fluid from the transgenic animal.

95. A method of claim 93, wherein the fluid is serum or milk.

96. A method of treating a disease or disease symptom in a patient, comprising the step of administering a fusion protein of claim 1 or claim 43.

97. The fusion protein of claim 1 or claim 43, wherein the Tf protein has a N-terminal domain at each end of the protein.

98. The fusion protein of claim 97, wherein the antibody variable region is fused to each N-terminal domain of the Tf protein.

99. The fusion protein of claim 1 or claim 43, wherein the antibody variable region binds specifically to a toxin.

100. A method of claim 96, wherein the antibody variable regions binds to TNF.

101. A method of claim 100, wherein the disease is selected from the group consisting of septic shock; endotoxic shock; cachexia syndromes associated with bacterial infections, viral infection, parasite infection, neoplastic disease; autoimmune disease, arthritis, and adverse effects associated with treatment for the prevention of graft rejection.
